(19) United States
(12) Patent Application Publication
(10) Pub. No.: US 2006/0205740 A1
(43) Pub. Date: Sep. 14, 2006

(54) Chemical compounds

(76) Inventors: Michael John Reno, Durham, NC (US); Kirk Lawrence Stevens, Durham, NC (US); Alex Gregory Waterson, Durham, NC (US); Yuemei Zhang, Montgomery, NJ (US)

Correspondence Address: GLAXOSMITHKLINE;CORPORATE INTELLECTUAL PROPERTY, MAI B475, FIVE MOORE DR., PO BOX 13398, RESEARCH TRIANGLE PARK, NC, 27709-3398, US

(21) Appl. No.: 10/568,052

(22) PCT Filed: Aug. 11, 2004
(86) PCT No.: PCT/US04/26251

§ 371 Date: Feb. 10, 2006

(30) Foreign Application Priority Data
Aug. 14, 2003 (US) 60495180

(51) Int. Cl. C07D 403/02; C07D 417/02; A61K 31/506

(52) U.S. Cl. 514/256; 544/296; 544/328

(57) ABSTRACT

The present invention discloses pyrimidine derivatives, compositions and medicaments containing the same, as well as processes for the preparation and use of such compounds, compositions and medicaments. Such pyrimidine derivatives are useful in the treatment of diseases associated with inappropriate ErbB family kinase.

### FIELD OF THE INVENTION

The present invention relates to pyrimidine derivatives, compositions and medicaments containing the same, as well as processes for the preparation and use of such compounds, compositions and medicaments. Such pyrimidine derivatives are useful in the treatment of diseases associated with inappropriate ErbB family kinase activity.

### BACKGROUND OF THE INVENTION

An important large family of enzymes is the protein kinase enzyme family. Currently, there are about 500 different known protein kinases. Protein kinases serve to catalyze the phosphorylation of an amino acid side chain in various proteins by the transfer of the γ-phosphate of the ATP-Mg²⁺ complex to said amino acid side chain. These enzymes control the majority of the signaling processes inside cells, thereby governing cell function, growth, differentiation and destruction (apoptosis) through reversible phosphorylation of the hydroxyl groups of serine, threonine and tyrosine residues in proteins. Studies have shown that protein kinases are key regulators of many cell functions, including signal transduction, transcriptional regulation, cell motility, and cell division. Several oncogenes have also been shown to encode protein kinases, suggesting that kinases play a role in oncogenesis. These processes are highly regulated, often by complex intermeshed pathways where each kinase will itself be regulated by one or more kinases. Consequently, aberrant or inappropriate protein kinase activity can contribute to the rise of disease states associated with such aberrant kinase activity. Due to their physiological relevance, variety and ubiquitousness, protein kinases have become one of the most important and widely studied family of enzymes In biochemical and medical research.

One type of protein kinases is protein tyrosine kinases (PTK). Aberrant PTK activity has been Implicated in a variety of disorders including psoriasis, rheumatoid arthritis, bronchitis, as well as cancer. Development of effective treatments for such disorders is a constant and ongoing enterprise in the medical field. The ErbB family of PTKs, which includes EGFR (c-ErbB-1), c-ErbB-2, and ErbB-4, is one group of PTKs that has attracted interest as a therapeutic target. Currently, of special interest, is the role of ErbB family PTKs in hyperproliferative disorders, particularly human malignancies. Elevated EGFR activity has, for example, been implicated in non-small cell lung, bladder, and head and neck cancers. Furthermore, increased c-ErbB-2 activity has been implicated in breast, ovarian, gastric and pancreatic cancers. Consequently, inhibition of ErbB family PTKs should provide a treatment for disorders characterized by aberrant ErbB family PTK activity. The biological role of ErbB family PTKs and their implication in various disease states is further discussed, for instance in U.S. Pat. No. 5,773,476; International Patent Application WO 99/35146; M. C. Hung et al, Seminars in Oncology, 26: 4, Suppl. 12 (August) 1999, 51-59; Ullrich et al, Cell, 61: 203-212, Apr. 20, 1990; Modjtahedi et al, Int'l. J. of Oncology, 13: 335-342, 1998; and J. R. Woodburn, Pharmacol. Ther., 82: 2-3, 241-250, 1999.

The present inventors have discovered novel pyrimidine compounds, which are inhibitors of erbB family kinase activity. Such derivatives are believed to be useful in the treatment of disorders associated with inappropriate erbB family kinase activity.

### SUMMARY OF THE INVENTION

In a first aspect of the present invention, there is provided a compound of Formula (I): 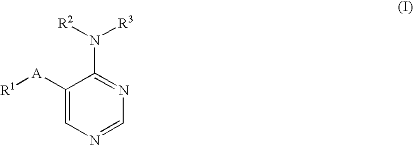
or a salt, solvate, or physiologically functional derivative thereof:
wherein:
A is C₁-C₄ alkenylene or C₁-C₄ alkynylene;
R is C₁-C₄ alkylene;
R¹ is the group defined by -(Z)-(Z¹)ₘ-(Z²)ₙ, wherein
Z is aryl, heteroaryl, heteroarylene, or arylene,
Z¹ is C(H)₂, where m is 0 or 1,
Z² is OR′, —SR′, —N(R′)R″, halo, C₁-C₃ alkyl, —CN, —C(O)R′, —C(O)N(R′)R″, or heterocyclyl, where n is 0 or 1;
   R′ is —H or C₁-C₃ alkyl;
   R″ is —H, —C(O)R′″, —C(S)R′″, —ROR′″, —C(═NH)N(R′)R″, C₁-C₃ alkyl, C₁-C₃ hydroxyalkyl, cyanoalkyl, —S(O)₂R′″, —RS(O)₂R′″, —C(O)N(R′)R′″, —C(O)N(R′)R^{a}, —C(O)RS(O)₂R′″, —C(O)ROROR′″, —C(O)RSR′″, —C(O)RNR′R″, —C(O)RC(O)OR′″, —RN(R′)RN(R′)C(O)R′″, heterocyclyl, or aralkyl;
   R′″ is C₁-C₃ alkyl, C₁-C₃ haloalkyl, C₁-C₃ hydroxyalkyl, cyanoalkyl, or aryl;
   R^{a} is —RS(O)₂R′, aralkyl, or —ROR′″;
   R² is —H or C₁-C₃ alkyl;
   R³ is the group defined by -(Q)-(Q¹)ᵣ-(Q²), wherein
Q is arylene or heteroarylene
Q¹ is O, where r is 0 or 1, and
Q² is aralkyl, heteroaryl, or aryl.

In a second aspect of the present invention, there is provided a compound of Formula (I): 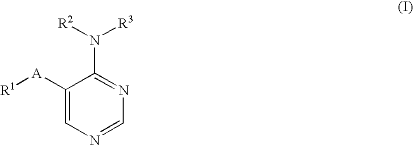
or a salt, solvate, or physiologically functional derivative thereof:
wherein:
A is C≡C;
R is C₁-C₄ alkylene;
R¹ is the group defined by -(Z)-(Z¹)ₘ-(Z²)ₙ, wherein
Z is aryl, heteroaryl, heteroarylene, or arylene,
Z¹ is C(H)₂, where m is 0 or 1,
Z² is OR′, —SR′, —N(R′)R″, halo, C₁-C₃ alkyl, —CN, —C(O)R′, —C(O)N(R′)R″, or heterocyclyl, where n is 0 or 1;
   R′ is —H or C₁-C₃ alkyl;
   R″ is —H, —C(O)R′″, —C(S)R′″, —ROR′″, —C(═NH)N(R′)R″, C₁-C₃ alkyl, C₁-C₃ hydroxyalkyl, cyanoalkyl, —S(O)₂R′″, —RS(O)₂R′″, —C(O)N(R′)R′″, —C(O)N(R′)R^{a}, —C(O)RS(O)₂R′″, —C(O)ROROR′″, —C(O)RSR′″, —C(O)RNR′R″, —C(O)RC(O)OR′″, —RN(R′)RN(R′)C(O)R′″, heterocyclyl, or aralkyl;
   R′″ is C₁-C₃ alkyl, C₁-C₃ haloalkyl, C₁-C₃ hydroxyalkyl, cyanoalkyl, or aryl;
   R^{a} is —RS(O)₂R′, aralkyl, or —ROR′″;
   R² is —H or C₁-C₃ alkyl;
   R³ is the group defined by -(Q)-(Q¹)ᵣ-(Q²), wherein
Q is arylene or heteroarylene
Q¹ is O, where r is 0 or 1, and
Q² is aralkyl, heteroaryl, or aryl.

In a third aspect of the present invention, there is provided a pharmaceutical composition comprising a therapeutically effective amount of a compound of formula (I) or a salt, solvate, or a physiologically functional derivative thereof and one or more of pharmaceutically acceptable carriers, diluents and excipients.

In a fourth aspect of the present invention, there is provided a method of treating a disorder in a mammal, said disorder being mediated by inappropriate activity of at least one erbB family kinase, comprising: administering to said mammal a therapeutically effective amount of a compound of formula (I) or a salt, solvate or a physiologically functional derivative thereof.

In a fifth aspect of the present invention, there is provided a method of treating a disorder in a mammal, said disorder being mediated by inappropriate activity of at least two erbB family kinases, comprising: administering to said mammal a therapeutically effective amount of a compound of formula (I) or a salt, solvate or a physiologically functional derivative thereof.

In a sixth aspect of the present invention, there is provided a compound of formula (I), or a salt, solvate, or a physiologically functional derivative thereof for use in therapy.

In a seventh aspect of the present invention, there is provided the use of a compound of formula (I), or a salt, solvate, or a physiologically functional derivative thereof in the preparation of a medicament for use in the treatment of a disorder mediated by inappropriate activity of at least one erbB family kinase.

In an eighth aspect of the present invention, there is provided the use of a compound of formula (I), or a salt, solvate, or a physiologically functional derivative thereof in the preparation of a medicament for use in the treatment of a disorder mediated by inappropriate activity of at least two erbB family kinases.

### DETAILED DESCRIPTION OF THE INVENTION

As used herein, the term “effective amount” means that amount of a drug or pharmaceutical agent that will elicit the biological or medical response of a tissue, system, animal or human that is being sought, for instance, by a researcher or clinician. Furthermore, the term “therapeutically effective amount” means any amount which, as compared to a corresponding subject who has not received such amount, results in improved treatment, healing, prevention, or amelioration of a disease, disorder, or side effect, or a decrease in the rate of advancement of a disease or disorder. The term also includes within its scope amounts effective to enhance normal physiological function.

As used herein the term “erbB family kinase” includes within its scope EGFR (erbB-1), erbB-2, and erbB-4.

As used herein the term “alkyl” refers to a straight- or branched-chain hydrocarbon radical having from one to twelve carbon atoms, optionally substituted with substituents selected from the group consisting of unsubstituted C₁-C₆ alkyl, C₁-C₆ hydroxyalkyl, C₁-C₆ alkoxy, C₁-C₆ alkylsulfanyl, C₁-C₆ alkylsulfenyl, C₁-C₆ alkylsulfonyl, oxo, hydroxy, mercapto, amino (optionally substituted by alkyl), carboxy, carbamoyl (optionally substituted by alkyl), aryl, aryloxy, heteroaryl, heterocyclyl, aminosulfonyl (optionally substituted by alkyl), nitro, cyano, halo, or C₁-C₆ perfluoroalkyl, multiple degrees of substitution being allowed. Examples of “alkyl” as used herein include, but are not limited to, methyl, ethyl, n-propyl, isopropyl, n-butyl, isobutyl, t-butyl, n-pentyl, isopentyl, and the like.

As used herein, the terms “C₁-C₃ alkyl” and “C₁-C₆ alkyl” refer to an alkyl group, as defined above, containing at least 1, and at most 3 or 6 carbon atoms respectively. Examples of such branched or straight-chained alkyl groups useful in the present invention include, but are not limited to, methyl, ethyl, n-propyl, isopropyl, n-butyl, isobutyl, t-butyl, n-pentyl, or isopentyl.

As used herein, the term “alkylene” refers to a straight or branched chain divalent hydrocarbon radical having from one to ten carbon atoms, optionally substituted with substituents selected from the group which includes C₁-C₆ alkyl, C₁-C₆ alkoxy, C₁-C₆ alkylsulfanyl, C₁-C₆ alkylsulfenyl, C₁-C₆ alkylsulfonyl, oxo, hydroxy, mercapto, amino (optionally substituted by alkyl), carboxy, carbamoyl (optionally substituted by alkyl), aryl, heteroaryl, heterocyclyl, aminosulfonyl (optionally substituted by alkyl), nitro, cyano, halo, and C₁-C₆ perfluoroalkyl, multiple degrees of substitution being allowed. Examples of “alkylene” as used herein include, but are not limited to, methylene, ethylene, n-propylene, n-butylene, and the like.

As used herein, the terms “C₁-C₃ alkylene” and “C₁-C₄ alkylene” refer to an alkylene group, as defined above, which contains at least 1, and at most 3 or 4, carbon atoms respectively. Examples of “C₁-C₃ alkylene” or “C₁-C₄ alkylene” groups useful in the present invention include, but are not limited to, methylene, ethylene, n-propylene, isopropylene, n-butylene and the like.

As used herein, the terms “C₁-C₃ haloalkyl” and “C₁-C₆ haloalkyl” refer to an alkyl group as defined above containing at least 1, and at most 3 or 6 carbon atoms respectively substituted with at least one halo group, halo being as defined herein. Examples of such branched or straight chained haloalkyl groups useful in the present invention include, but are not limited to, methyl, ethyl, propyl, isopropyl, isobutyl, n-butyl, n-pentyl, substituted independently with one or more halos, e.g., fluoro, chloro, bromo and iodo.

As used herein, the term “C₁-C₃ hydroxyalkyl” refers to an alkyl group as defined above containing at least 1, and at most 3 carbon atoms substituted with at least one hydroxy group, hydroxy being as defined herein. Examples of such branched or straight chained hydroxyalkyl groups useful in the present invention include, but are not limited to, methyl, ethyl, propyl, or isopropyl, substituted independently with one or more hydroxy groups.

As used herein, the term “cycloalkyl” refers to a non-aromatic cyclic hydrocarbon ring containing from 3 to 10 carbon atoms and which optionally Includes a C₁-C₃ alkylene linker through which it may be attached. In a like manner the term “C₃-C₇ cycloalkyl” refers to a non-aromatic cyclic hydrocarbon ring having from three to seven carbon atoms optionally substituted with substituents selected from the group which includes C₁-C₆ alkyl, C₁-C₆ alkoxy, C₁-C₆ alkylsulfanyl, C₁-C₆ alkylsulfenyl, C₁-C₆ alkylsulfonyl, oxo, hydroxy, mercapto, amino (optionally substituted by alkyl), carboxy, carbamoyl (optionally substituted by alkyl), aminosulfonyl (optionally substituted by alkyl), nitro, cyano, halo, C₁-C₆ perfluoroalkyl, multiple degrees of substitution being allowed and which optionally includes a C₁-C₃ alkylene linker through which it may be attached. The C₁-C₃ alkylene group is as defined above. Exemplary “C₃-C₇ cycloalkyl” groups useful in the present invention include, but are not limited to, cyclopropyl, cyclobutyl, cyclopentyl, cyclohexyl and cycloheptyl.

As used herein, the term “C₃-C₇ cycloalkylene” refers to a non-aromatic alicyclic divalent hydrocarbon radical having from three to seven carbon atoms, optionally substituted with substituents selected from the group which includes C₁-C₆ alkyl, C₁-C₆ alkoxy, C₁-C₆ alkylsulfanyl, C₁-C₆ alkylsulfenyl, C₁-C₆ alkylsulfonyl, oxo, hydroxy, mercapto, amino (optionally substituted by alkyl), carboxy, carbamoyl (optionally substituted by alkyl), aminosulfonyl (optionally substituted by alkyl), nitro, cyano, halo, C₁-C₆ perfluoroalkyl, multiple degrees of substitution being allowed and which optionally includes 1 or 2 C₁-C₃ alkylene linker(s) through which it may be attached at one or two points. Examples of “cycloalkylene” as used herein include, but are not limited to, cyclopropyl-1,1-diyl, cyclopropyl-1,2-diyl, cyclobutyl-1,2-diyl, cyclopentyl-1,3-diyl, cyclohexyl-1,4-diyl, cycloheptyl-1,4-diyl, or cyclooctyl-1,5-diyl, and the like.

As used herein, the term “alkenylene” refers to an straight or branched chain divalent hydrocarbon radical having from two to ten carbon atoms and one or more carbon-carbon double bonds, optionally substituted with substituents selected from the group which includes C₁-C₆ alkyl, C₁-C₆ alkoxy, C₁-C₆ alkylsulfanyl, C₁-C₆ alkylsulfenyl, C₁-C₆ alkylsulfonyl, oxo, hydroxy, mercapto, amino (optionally substituted by alkyl), carboxy, carbamoyl (optionally substituted by alkyl), aminosulfonyl (optionally substituted by alkyl), nitro, cyano, halogen and C₁-C₆ perfluoroalkyl, multiple degrees of substitution being allowed. Examples of “alkenylene” as used herein include, but are not limited to, ethene-1,2-diyl, propene-1,3-diyl, butene-1,4-diyl, and the like.

As used herein, the term “C₁-C₄ alkenylene” refers to an alkenylene group as defined above containing at least 1, and at most 4, carbon atoms. Examples of “C₁-C₄ alkenylene” groups useful in the present invention include, but are not limited to, ethene-1,2-diyl, propene-1,3-diyl, butene-1,4-diyl, and the like.

As used herein, the term “alkynylene” refers to a straight or branched chain divalent hydrocarbon radical having from two to ten carbon atoms and one or more carbon-carbon triple bonds, optionally substituted with substituents selected from the group which includes C₁-C₆ alkyl, C₁-C₆ alkoxy, C₁-C₆ alkylsulfanyl, C₁-C₆ alkylsulfenyl, C₁-C₆ alkylsulfonyl, oxo, hydroxy, mercapto, amino (optionally substituted by alkyl), carboxy, carbamoyl (optionally substituted by alkyl), aminosulfonyl (optionally substituted by alkyl), nitro, cyano, halogen and C₁-C₆ perfluoroalkyl, multiple degrees of substitution being allowed. Examples of “alkynylene” as used herein include, but are not limited to, ethyne-1,2-diyl, propyne-1,3-diyl, and the like.

As used herein, the term “C₁-C₄ alkynylene” refers to an alkynylene group as defined above containing at least 1, and at most 4 carbon atoms. Examples of “C₁-C₄ alkynylene” groups useful in the present invention include, but are not limited to, ethyne-1,2-diyl, propyne-1,3-diyl, and the like.

As used herein, the term “halogen” refers to fluorine (F), chlorine (Cl), bromine (Br), or iodine (I) and the term “halo” refers to the halogen radicals: fluoro (—F), chloro (—Cl), bromo (—Br), and iodo (—I).

As used herein, the term “hydroxy” refers to the group —OH.

As used herein, the term “heterocyclic” or the term “heterocyclyl” refers to a three to twelve-membered non-aromatic heterocyclic ring, being saturated or having one or more degrees of unsaturation, atoms containing one or more heteroatom substitutions selected from S, S(O), S(O)₂, O, or N atoms and which optionally includes a C₁-C₃ alkylene linker through which it may be attached and is optionally substituted with substituents selected from the group consisting of C₁-C₆ alkyl, C₁-C₆ alkoxy, C₁-C₆ alkylsulfanyl, C₁-C₆ alkylsulfenyl, C₁-C₆ alkylsulfonyl, oxo, hydroxy, mercapto, amino (optionally substituted by alkyl), carboxy, carbamoyl (optionally substituted by alkyl), aminosulfonyl (optionally substituted by alkyl), nitro, cyano, halo, aryl, aralkyl, heteroaryl, or C₁-C₆ perfluoroalkyl, multiple degrees of substitution being allowed. Such a ring may be optionally fused to one or more other “heterocyclic” ring(s) or cycloalkyl ring(s). Examples of “heterocyclic” moieties include, but are not limited to, tetrahydrofuranyl, pyranyl, 1,4-dioxanyl, 1,3-dioxanyl, piperidinyl, piperazinyl, 2,4-piperazinedionyl, pyrrolidinyl, imidazolidinyl, pyrazolidinyl, morpholinyl, thiomorpholinyl, tetrahydrothiopyranyl tetrahydrothiophenyl, and the like.

As used herein, the term “aryl” refers to an optionally substituted benzene ring or to an optionally substituted benzene ring system fused to one or more optionally substituted benzene rings, one or more cycloalkyl ring(s), or one or more heterocyclyl rings to form, for example, anthracene, phenanthrene, napthalene, or indan ring systems. Exemplary optional substituents include C₁-C₆ alkyl, C₁-C₆ alkoxy, C₁-C₆ haloalkyl, C₁-C₆ haloalkoxy, C₁-C₆ alkylsulfanyl, C₁-C₆ alkylsulfenyl, C₁-C₆ alkylsulfonyl, C₁-C₆ alkylsulfonylamino, arylsulfonoamino, alkylcarboxy, alkylcarboxyamide, oxo, hydroxy, mercapto, amino (optionally substituted by alkyl), carboxy, tetrazolyl, carboxamide, carbamoyl (optionally substituted by alkyl), aminosulfonyl (optionally substituted by alkyl), ureido, arylurea, arylthiourea, alkylurea, cycloalkylurea, sulfonylurea, acyl, aroyl, aroylamino, heteroaroyl, acyloxy, aroyloxy, heteroaroyloxy, alkoxycarbonyl, nitro, cyano, halo, heteroaryl, heterocyclyl, aryl, aryloxy, or aralkoxy, multiple degrees of substitution being allowed. Examples of “aryl” groups include, but are not limited to, indanyl, phenyl, 2-naphthyl, 1-naphthyl, biphenyl, as well as substituted derivatives thereof.

As used herein, the term “arylene” refers to a benzene ring diradical or to a benzene ring system diradical fused to one or more optionally substituted benzene rings, optionally substituted with substituents selected from the group which includes C₁-C₆ alkyl, C₁-C₆ alkoxy, aryloxy, heteroaryloxy, C₁-C₆ alkylsulfanyl, C₁-C₆ alkylsulfenyl, C₁-C₆ alkylsulfonyl, oxo, hydroxy, mercapto, amino (optionally substituted by alkyl), carboxy, tetrazolyl, carbamoyl (optionally substituted by alkyl), aminosulfonyl (optionally substituted by alkyl), ureido, arylurea, arylthiourea, alkylurea, cycloalkylurea, sulfonylurea, acyl, aroyl, heteroaroyl, acyloxy, aroyloxy, heteroaroyloxy, alkoxycarbonyl, nitro, cyano, halo, C₁-C₆ perfluoroalkyl, heterocyclyl, heteroaryl and aryl, multiple degrees of substitution being allowed. Examples of “arylene” include, but are not limited to, benzene-1,4-diyl, naphthalene-1,8-diyl, anthracene-1,4-diyl, and the like.

As used herein, the term “aralkyl” refers to an aryl or heteroaryl group, as defined herein, attached through a C₁-C₃ alkylene linker, wherein the C₁-C₃ alkylene is as defined herein. Examples of “aralkyl” include, but are not limited to, benzyl, phenylpropyl, 2-pyridylmethyl, 3-isoxazolylmethyl, 5-methyl-3-isoxazolylmethyl, and 2-imidazolyl ethyl.

As used herein, the term “heteroaryl” refers to a monocyclic five to seven membered aromatic ring, or to a fused bicyclic or tricyclic aromatic ring system comprising two of such monocyclic five to seven membered aromatic rings. These heteroaryl rings contain one or more nitrogen, sulfur, and/or oxygen heteroatoms, where N-oxides and sulfur oxides and dioxides are permissible heteroatom substitutions and may be optionally substituted with up to three members selected from a group consisting of C₁-C₆ alkyl, C₁-C₆ alkoxy, C₁-C₆ haloalkyl, C₁-C₆ haloalkoxy, C₁-C₆ alkylsulfanyl, C₁-C₆ alkylsulfenyl, C₁-C₆ alkylsulfonyl, C₁-C₆ alkylsulfonylamino, arylsulfonoamino, alkylcarboxy, alkylcarboxyamide, oxo, hydroxy, mercapto, amino (optionally substituted by alkyl), carboxy, tetrazolyl, carboxamide, carbamoyl (optionally substituted by alkyl), aminosulfonyl (optionally substituted by alkyl), ureido, arylurea, arylthiourea, alkylurea, cycloalkylurea, sulfonylurea, acyl, aroyl, aroylamino, heteroaroyl, acyloxy, aroyloxy, heteroaroyloxy, alkoxycarbonyl, nitro, cyano, halo, heteroaryl, heterocyclyl, aryl, aryloxy, or aralkoxy, multiple degrees of substitution being allowed. Examples of “heteroaryl” groups used herein include furanyl, thiophenyl, pyrrolyl, imidazolyl, pyrazolyl, triazolyl, tetrazolyl, thiazolyl, oxazolyl, isoxazolyl, oxadiazolyl, oxo-pyridyl, thiadiazolyl, isothiazolyl, pyridyl, pyridazyl, pyrazinyl, pyrimidyl, quinazolinyl, quinolinyl, isoquinolinyl, benzofuranyl, benzothiophenyl, indolyl, indazolyl, and substituted versions thereof.

As used herein, the term “heteroarylene” refers to a five- to seven-membered aromatic ring diradical, or to a polycyclic heterocyclic aromatic ring diradical, containing one or more nitrogen, oxygen, or sulfur heteroatoms, where N-oxides and sulfur monoxides and sulfur dioxides are permissible heteroaromatic substitutions, optionally substituted with substituents selected from the group consisting of: C₁-C₆ alkyl, C₁-C₆ alkoxy, aryloxy, heteroaryloxy, C₁-C₆ alkylsulfanyl, C₁-C₆ alkylsulfenyl, C₁-C₆ alkylsulfonyl, oxo, hydroxy, mercapto, amino (optionally substituted by alkyl), carboxy, tetrazolyl, carbamoyl (optionally substituted by alkyl), aminosulfonyl (optionally substituted by alkyl), ureido, arylurea, arylthiourea, alkylurea, cycloalkylurea, sulfonylurea, acyl, aroyl, heteroaroyl, acyloxy, aroyloxy, heteroaroyloxy, alkoxycarbonyl, nitro, cyano, halo, C₁-C₆ perfluoroalkyl, heterocyclyl, heterocyclic spiro ring system, heteroaryl, or aryl, multiple degrees of substitution being allowed. For polycyclic aromatic ring system diradicals, one or more of the rings may contain one or more heteroatoms. Examples of “heteroarylene” used herein are furan-2,5-diyl, thiophene-2,4-diyl, 1,3,4-oxadiazole-2,5-diyl, 1,3,4 thiadiazole-2,5-diyl, 1,3-thiazole-2,4-diyl, 1,3-thiazole-2,5-diyl, pyridine-2,4-diyl, pyridine-2,3-diyl, pyridine-2,5-diyl, pyrimidine-2,4-diyl, quinoline-2,3-diyl, and the like.

As used herein, the term “alkoxy” refers to the group RₐO—, where Rₐ is alkyl as defined above and the terms “C₁-C₃ alkoxy” and “C₁-C₆ alkoxy” refer to an alkoxy group as defined herein wherein the alkyl moiety contains at least 1, and at most 3 or 6, carbon atoms. Exemplary “C₁-C₃ alkoxy” and “C₁-C₆ alkoxy” groups useful in the present invention include, but are not limited to, methoxy, ethoxy, n-propoxy, isopropoxy, n-butoxy, and t-butoxy.

As used herein, the term “amino” refers to the group —NH₂.

As used herein the term “alkylamino” refers to the group —NHRₐ wherein Rₐ is alkyl as defined above.

As used herein the term “arylamino” refers to the group —NHRₐ wherein Rₐ is aryl as defined above.

As used herein the term “aralkylamino” refers to the group —NHRₐ wherein Rₐ is an aralkyl group as defined above.

As used herein the term “aralkoxy” refers to the group R_{b}RₐO—, where Rₐ is alkylene and R_{b} is aryl or heteroaryl all as defined above.

As used herein the term “aryloxy” refers to the group RₐO—, where Rₐ is aryl or heteroaryl both as defined above.

As used herein the term “ureido” refers to the group —NHC(O)NH₂

As used herein, the term “arylurea” refers to the group —NHC(O)NHRₐR_{b} wherein Rₐ is aryl or heteroaryl and R_{b} is —H, alkyl, or aryl as defined above.

As used herein, the term “arylthiourea” refers to the group —NHC(S)NHRₐ wherein Rₐ is aryl as defined above.

As used herein, the term “alkylurea” refers to the group —NHC(O)NRₐR_{b} wherein Rₐ is alkyl and R_{b} is —H or alkyl as defined above.

As used herein, the term “cycloalkylurea” refers to the group —NHC(O)NHRₐ wherein Rₐ is cycloalkyl as defined above.

As used herein, the term “haloalkoxy” refers to the group RₐO—, where Rₐ is haloalkyl as defined above and the term “C₁-C₆ haloalkoxy” refers to a haloalkoxy group as defined herein wherein the haloalkyl moiety contains at least 1, and at most 6, carbon atoms. Exemplary C₁-C₆ haloalkoxy groups useful in the present invention include, but are not limited to, trifluoromethoxy.

As used herein, the term “alkylsulfanyl” refers to the group RₐS—, where Rₐ is alkyl as defined above and the term “C₁-C₆ alkylsulfanyl” refers to an alkylsulfanyl group as defined herein wherein the alkyl moiety contains at least 1, and at most 6, carbon atoms.

As used herein, the term “haloalkylsulfanyl” refers to the group RₐS—, where Rₐ is haloalkyl as defined above and the term “C₁-C₆ haloalkylsulfanyl” refers to a haloalkylsulfanyl group as defined herein wherein the alkyl moiety contains at least 1, and at most 6, carbon atoms.

As used herein, the term “alkylsulfenyl” refers to the group RₐS(O)—, where Rₐ is alkyl as defined above and the term “C₁-C₆ alkylsulfenyl” refers to an alkylsulfenyl group as defined herein wherein the alkyl moiety contains at least 1, and at most 6, carbon atoms.

As used herein, the term “alkylsulfonyl” refers to the group RₐS(O)₂—, where Rₐ is alkyl as defined above and the term “C₁-C₆ alkylsulfonyl” refers to an alkylsulfonyl group as defined herein wherein the alkyl moiety contains at least 1, and at most 6, carbon atoms.

As used herein, the term “alkylsulfonylamino” refers to the group —NR_{b}S(O)₂Rₐ wherein Rₐ is alkyl and R_{b} is —H or C₁-C₆ alkyl as defined above, and the term “C₁-C₆ alkylsulfonylamino” refers to an alkylsulfonylamino group as defined herein wherein the alkyl moiety contains at least 1, and at most 6, carbon atoms.

As used herein, the term “arylsulfonylamino” refers to the group —NR_{b}S(O)₂Rₐ wherein Rₐ is aryl or heteroaryl and R_{b} is —H or C₁-C₆ alkyl as defined above.

As used herein, the term “alkylcarboxyamide” refers to the group —NHC(O)Rₐ wherein Rₐ is alkyl, amino, or amino substituted with alkyl, aryl or heteroaryl as described above.

As used herein the term “alkylcarboxy” refers to the group —C(O)Rₐ wherein Rₐ is alkyl as described above.

As used herein, the term “oxo” refers to the group ═O.

As used herein, the term “mercapto” refers to the group —SH.

As used herein, the term “carboxy” refers to the group —C(O)ORₐ, wherein Rₐ is H or alkyl as defined herein.

As used herein, the term “cyano” refers to the group —CN.

As used herein the term “cyanoalkyl” refers to the group —RₐCN wherein Rₐ is alkyl as defined above. Exemplary “cyanoalkyl” groups useful in the present invention include, but are not limited to, cyanomethyl, cyanoethyl, and cyanoisopropyl.

As used herein, the term “aminosulfonyl” refers to the group —S(O)₂NRₐR_{b} wherein Rₐ and R_{b} are independently H, C₁-C₆alkyl, aryl, aralkyl, or heteroaryl.

As used herein, the term “carbamoyl” refers to the group —OC(O)NHRₐ, where Rₐ is hydrogen or alkyl as defined herein.

As used herein, the term “carboxamide” refers to the group —C(O)NRₐR_{b} wherein Rₐ and R_{b} are independently H, C₁-C₆alkyl, aryl, aralkyl, or heteroaryl.

As used herein, the term “sulfanyl” shall refer to the group —S—.

As used herein, the term “sulfenyl” shall refer to the group —S(O)—.

As used herein, the term “sulfonyl” shall refer to the group —S(O)₂— or —SO₂—.

As used herein, the term “acyl” refers to the group RₐC(O)—, where Rₐ is alkyl, cycloalkyl, or heterocyclyl as defined herein.

As used herein, the term “aroyl” refers to the group RₐC(O)—, where Rₐ is aryl as defined herein.

As used herein, the term “aroylamino” refers to the group RₐC(O)NH—, where Rₐ is aryl as defined herein.

As used herein, the term “heteroaroyl” refers to the group RₐC(O)—, where Rₐ is heteroaryl as defined herein.

As used herein, the term “alkoxycarbonyl” refers to the group RₐOC(O)—, where Rₐ is alkyl as defined herein.

As used herein, the term “acyloxy” refers to the group RₐC(O)O—, where Rₐ is alkyl, cycloalkyl, or heterocyclyl as defined herein.

As used herein, the term “aroyloxy” refers to the group RₐC(O)O—, where Rₐ is aryl as defined herein.

As used herein, the term “heteroaroyloxy” refers to the group RₐC(O)O—, where Rₐ is heteroaryl as defined herein.

As used herein, the term “optionally” means that the subsequently described event(s) may or may not occur, and includes both event(s), which occur, and events that do not occur.

As used herein, the term “physiologically functional derivative” refers to any pharmaceutically acceptable derivative of a compound of the present invention, for example, an ester or an amide, which upon administration to a mammal is capable of providing (directly or indirectly) a compound of the present invention or an active metabolite thereof. Such derivatives are clear to those skilled in the art, without undue experimentation, and with reference to the teaching of Burger's Medicinal Chemistry And Drug Discovery, 5^{th} Edition, Vol 1: Principles and Practice, which is incorporated herein by reference to the extent that it teaches physiologically functional derivatives.

As used herein, the term “solvate” refers to a complex of variable stoichiometry formed by a solute (in this invention, a compound of formula (I) or a salt or physiologically functional derivative thereof) and a solvent. Such solvents for the purpose of the invention may not interfere with the biological activity of the solute. Examples of suitable solvents include, but are not limited to, water, methanol, ethanol and acetic acid. Preferably the solvent used is a pharmaceutically acceptable solvent. Examples of suitable pharmaceutically acceptable solvents include, without limitation, water, ethanol and acetic acid. Most preferably the solvent used is water.

As used herein, the term “substituted” refers to substitution with the named substituent or substituents, multiple degrees of substitution being allowed unless otherwise stated.

Certain of the compounds described herein may contain one or more chiral atoms, or may otherwise be capable of existing as two enantiomers. The compounds of this invention include mixtures of enantiomers as well as purified enantiomers or enantiomerically enriched mixtures. Also included within the scope of the invention are the individual isomers of the compounds represented by formula (I) above as well as any wholly or partially equilibrated mixtures thereof. The present invention also covers the individual isomers of the compounds represented by the formulas above as mixtures with isomers thereof in which one or more chiral centers are inverted. Also, it is understood that any tautomers and mixtures of tautomers of the compounds of formula (I) are included within the scope of the compounds of formula (I).

It is to be understood that reference to compounds of formula (I), above, following herein, refers to compounds within the scope of formula I, as defined above with respect to A, m, n, r, R, R′, R″, R′″, R¹, R², R³, R⁴, R⁵, R^{a}, Z, Z¹, Z², Q, Q¹, and Q² unless specifically limited otherwise.

It is also understood that substituent bonding locations having an unfilled valence are indicated by 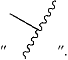
The appropriate attachments are further illustrated in the working examples recited below.

In one embodiment, A is C₁-C₄ alkynylene. In one embodiment A is C═C. In another embodiment A is C₁-C₄ alkenylene. In one embodiment, A is C═C.

As recited above, R¹ is the group defined by -(Z)-(Z¹)ₘ-(Z²)ₙ. In one embodiment, Z is heteroaryl and m and n are each 0. In another embodiment, Z is heteroaryl and m is 0 and n is 0, where the heteroaryl group is selected from

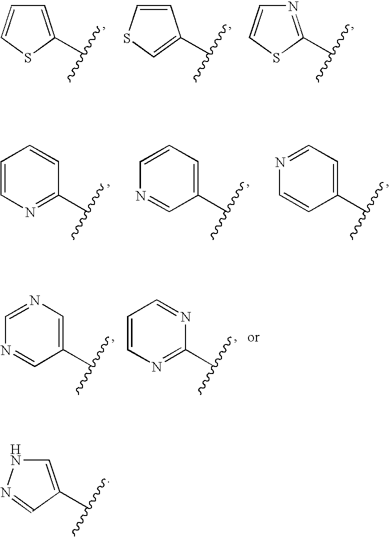

In an alternative embodiment, Z is heteroarylene, m is 0 or 1, n is 1, and Z¹ and Z² are as defined above. In an alternative embodiment, Z is heteroarylene selected from 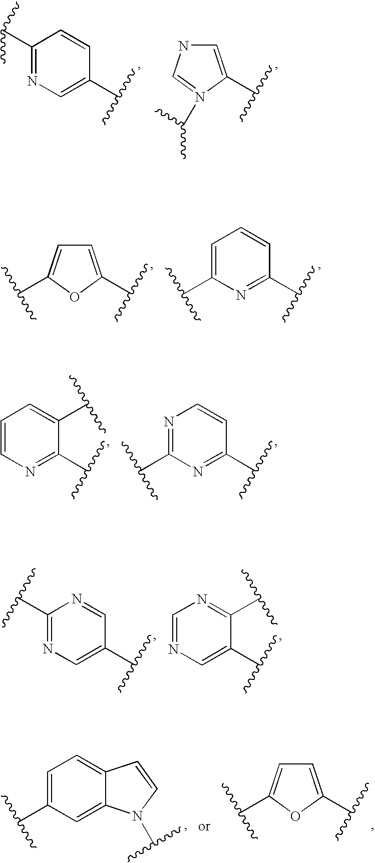
where m is 0 or 1, n is 1, and Z¹ and Z² are as defined above.

In another embodiment, Z is aryl, and m and n are each 0. In one embodiment, Z is

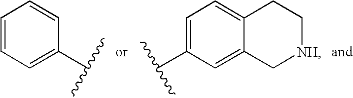

m and n are 0.

In an alternative embodiment, Z is arylene, m is 0 or 1, n is 1, and Z¹ and Z² are as defined above. In an alternative embodiment, Z is 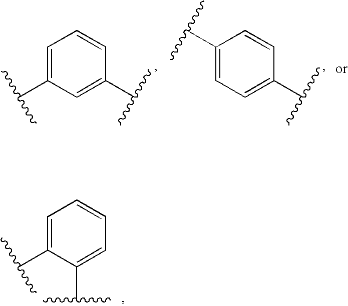
and m is 0 or 1, n is 1, and Z¹ and Z² are as defined above.

In one embodiment, R² is —H. In another embodiment, R² is C₁-C₃ alkyl.

As recited above, R³ is the group defined by -(Q)-(Q¹)ᵣ-(Q²). In one embodiment, Q is arylene, Q¹ is O and r is 1, and Q² is aralkyl, aryl, or heteroaryl. In another embodiment, Q is 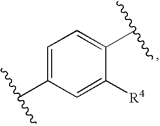
wherein R⁴ is —H or halo, preferably —Cl or —F, Q¹ is O and r is 1, and Q² is selected from 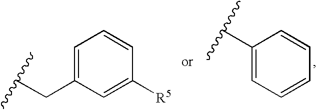
wherein R⁵ is halo, preferably —F, —Cl, or —Br.

In an alternative embodiment, Q is arylene, r is 0, and Q² is aralkyl. In one embodiment, Q is selected from 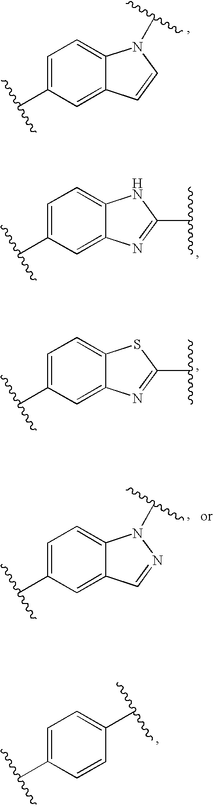
r is 0, and Q² is selected from

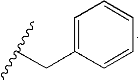

Specific examples of compounds of the present invention include the following:
2-benzyl-N-{5-[(E)-2-phenylethenyl]pyrimidin-4-yl}-1H-benzimidazol-5-amine;
2-benzyl-N-{5-[(E)-2-thien-3-ylethenyl]pyrimidin-4-yl}-1H-benzimidazol-5-amine;
2-benzyl-N-{5-[(E)-2-(1H-pyrazol-4-yl)ethenyl]pyrimidin-4-yl}-1H-benzimidazol-5-amine;
3-((E)-2-{4-[(2-benzyl-1H-benzimidazol-5-yl)amino]pyrimidin-5-yl}ethenyl)-N-methylbenzamide;
2-benzyl-N-{5-[(E)-2-thien-3-ylethenyl]pyrimidin-4-yl}-1,3-benzothiazol-5-amine;
1-benzyl-N-{5-[(E)-2-pyridin-3-ylethenyl]pyrimidin-4-yl}-1H-indazol-5-amine;
1-benzyl-N-{5-[(E)-2-pyridin-4-ylethenyl]pyrimidin-4-yl}-1H-indazol-5-amine;
2-((E)-2-{4-[(1-benzyl-1H-indazol-5-yl)amino]pyrimidin-5-yl}ethenyl)pyridin-3-ol;
1-benzyl-N-{5-[(E)-2-(1H-pyrazol-4-yl)ethenyl]pyrimidin-4-yl}-1H-indazol-5-amine;
N-{5-[(E)-2-(2-aminopyrimidin-5-yl)ethenyl]pyrimidin-4-yl}-1-benzyl-1H-indazol-5-amine;
N-[3-((E)-2-{4-[(1-benzyl-1H-indazol-5-yl)amino]pyrimidin-5-yl}ethenyl)phenyl]acetamide;
N-(4-phenoxyphenyl)-5-[(E)-2-phenylethenyl]pyrimidin-4-amine;
N-{3-chloro-4-[(3-fluorobenzyl)oxy]phenyl}-5-[(E)-2-pyridin-3-ylethenyl]pyrimidin-4-amine;
N-{3-chloro-4-[(3-fluorobenzyl)oxy]phenyl}-5-[(E)-2-pyridin-4-ylethenyl]pyrimidin-4-amine;
2-{(E)-2-[4-({3-chloro-4-[(3-fluorobenzyl)oxy]phenyl}amino)pyrimidin-5-yl]ethenyl}pyridin-3-ol;
N-{3-chloro-4-[(3-fluorobenzyl)oxy]phenyl}-5-[(E)-2-thien-2-ylethenyl]pyrimidin-4-amine;
N-{3-chloro-4-[(3-fluorobenzyl)oxy]phenyl}-5-[(E)-2-thien-3-yletheny]pyrimidin-4-amine;
5{(E)-2-[4-({3-chloro-4-[(3-fluorobenzyl)oxy]phenyl}amino)pyrimidin-5-yl]ethenyl}pyrimidin-2-amine;
N-{3-chloro-4-[(3-fluorobenzyl)oxy]phenyl}-5-[(E)-2-(1H-pyrazol-4-yl)ethenyl]pyrimidin-4-amine;
N-(3-{(E)-2-[4-({3-chloro-4-[(3-fluorobenzyl)oxy]phenyl}amino)pyrimidin-5-yl]ethenyl}phenyl)acetamide;
N-{3-chloro-4-[(3-fluorobenzyl)oxy]phenyl}-5-[(E)-2-(3,4-dimethoxyphenyl) ethenyl]pyrimidin-4-amine;
N-{3-chloro-4-[(3-fluorobenzyl)oxy]phenyl}-5-[(E)-2-phenylethenyl]pyrimidin-4-amine;
N-(5-{(E)-2-[4-({3-chloro-4-[(3-fluorobenzyl)oxy]phenyl}amino)pyrimidin-5-yl]ethenyl}pyridin-2-yl)acetamide;
N-{3-chloro-4-[(3-fluorobenzyl)oxy]phenyl}-5-(thien-2-ylethynyl)pyrimidin-4-amine;
N-{3-chloro-4-[(3-fluorobenzyl)oxy]phenyl}-5-(pyridin-3-ylethynyl)pyrimidin-4-amine;
N-{3-chloro-4-[(3-fluorobenzyl)oxy]phenyl}-5-[(1-methyl-1H-imidazol-5-yl)ethynyl]pyrimidin-4-amine;
N-{3-chloro-4-[(3-fluorobenzyl)oxy]phenyl}-5-(1H-pyrazol-4-ylethynyl)pyrimidin-4-amine;
N-{3-chloro-4-[(3-fluorobenzyl)oxy]phenyl}-5-(pyrimidin-5-ylethynyl)pyrimidin-4-amine;
N-{3-chloro-4-[(3-fluorobenzyl)oxy]phenyl}-5-(1,3-thiazol-2-ylethynyl)pyrimidin-4-amine;
N-{3-chloro-4-[(3-fluorobenzyl)oxy]phenyl}-5-(thien-3-ylethynyl)pyrimidin-4-amine;
N-{3-chloro-4-[(3-fluorobenzyl)oxy]phenyl}-5-[(2-morpholin-4-ylpyrimidin-4-yl)ethynyl]pyrimidin-4-amine;
N-(3-chloro-4-{[(3-fluorophenyl)methyl]oxy}phenyl)-5-(2-pyrimidinylethynyl)-4-pyrimidinamine;
5-[(6-amino-3-pyridinyl)ethynyl]-N-(3-chloro-4-{[(3-fluorophenyl)methyl]oxy}phenyl)-4-pyrimidinamine;
N-{3-chloro-4-[(3-fluorobenzyl)oxy]phenyl}-5-[(3-fluorophenyl)ethynyl]pyrimidin-4-amine;
4-{[4-({3-chloro-4-[(3-fluorobenzyl)oxy]phenyl}amino)pyrimidin-5-yl]ethynyl}phenol;
N-{3-chloro-4-[(3-fluorobenzyl)oxy]phenyl}-5-[(6-methoxypyridin-2-yl)ethynyl]pyrimidin-4-amine;
5-[(3-aminophenyl)ethynyl]-N-{3-chloro-4-[(3-fluorobenzyl)oxy]phenyl}pyrimidin-4-amine;
N-(3-{[4-({3-chloro-4-[(3-fluorobenzyl)oxy]phenyl}amino)pyrimidin-5-yl]ethynyl}phenyl)acetamide;
N-(3-{[4-({3-chloro-4-[(3-fluorobenzyl)oxy]phenyl}amino)pyrimidin-5-yl]ethynyl}phenyl)ethanethioamide;
2-{[4-({3-chloro-4-[(3-fluorobenzyl)oxy]phenyl}amino)pyrimidin-5-yl]ethynyl}benzonitrile;
3-{[4-({3-chloro-4-[(3-fluorobenzyl)oxy]phenyl}amino)pyrimidin-5-yl]ethynyl}benzonitrile;
3-{[4-({3-chloro-4-[(3-fluorobenzyl)oxy]phenyl}amino)pyrimidin-5-yl]ethynyl}benzaldehyde;
N-{3-chloro-4-[(3-fluorobenzyl)oxy]phenyl}-5-(phenylethynyl)pyrimidin-4-amine;
N-{3-chloro-4-[(3-fluorobenzyl)oxy]phenyl}-5-(pyridin-2-ylethynyl)pyrimidin-4-amine;
5-[(4-aminophenyl)ethynyl]-N-{3-chloro-4-[(3-fluorobenzyl)oxy]phenyl}pyrimidin-4-amine;
N-(3-{[4-({3-chloro-4-[(3-fluorobenzyl)oxy]phenyl}amino)pyrimidin-5-yl]ethynyl}phenyl)-3-(methylthio)propanamide;
N-{3-chloro-4-[(3-fluorobenzyl)oxy]phenyl}-5-({1-[(4-methylphenyl)sulfonyl]-1H-indol-6-yl}ethynyl)pyrimidin-4-amine;
tert-butyl-3-{[4-({3-chloro-4-[(3-fluorobenzyl)oxy]phenyl}amino)pyrimidin-5-yl]ethynyl}benzylcarbamate;
N-(3-{[4-({3-chloro-4-[(3-fluorobenzyl)oxy]phenyl}amino)pyrimidin-5-yl]ethynyl}phenyl)guanidine;
N-(3-{[4-({3-chloro-4-[(3-fluorobenzyl)oxy]phenyl}amino)pyrimidin-5-yl]ethynyl}benzyl)acetamide;
N-{3-chloro-4-[(3-fluorobenzyl)oxy]phenyl}-5-{[3-({[2-(methylsulfonyl) ethyl]amino}methyl)phenyl]ethynyl}pyrimidin-4-amine;
5-{[4-({3-chloro-4-[(3-fluorobenzyl)oxy]phenyl}amino)pyrimidin-5-yl]ethynyl}-2-furaldehyde;
3-{[(5-{[4-({3-chloro-4-[(3-fluorobenzyl)oxy]phenyl}amino)pyrimidin-5-yl]ethynyl}-2-furyl)methyl]amino}propanenitrile;
(5-{[4-({3-chloro-4-[(3-fluorobenzyl)oxy]phenyl}amino)pyrimidin-5-yl]ethynyl}-2-furyl)methanol;
(4-{[4-({3-chloro-4-[(3-fluorobenzyl)oxy]phenyl}amino)pyrimidin-5-yl]ethynyl}-1,3-thiazol-2-yl)methanol;
N-{3-chloro-4-[(3-fluorobenzyl)oxy]phenyl}-5-(1,2,3,4-tetrahydro-isoquinolin-7-ylethynyl)pyrimidin-4-amine;
2-{[4-({3-chloro-4-[(3-fluorobenzyl)oxy]phenyl}amino)pyrimidin-5-yl]ethynyl}benzaldehyde;
N-{3-chloro-4-[(3-fluorobenzyl)oxy]phenyl}-5-{[5-({[2-(methylsulfonyl)ethyl]amino}methyl)-2-furyl]ethynyl}pyrimidin-4-amine;
N-(3-{[4-({3-chloro-4-[(3-fluorobenzyl)oxy]phenyl}amino)pyrimidin-5-yl]ethynyl}phenyl)-2-(2-methoxyethoxy)acetamide;
N-[3-({4-[(2-benzyl-1H-benzimidazol-5-yl)amino]pyrimidin-5-yl}ethynyl)phenyl]acetamide;
N¹-(3-{[4-({3-chloro-4-[(3-fluorobenzyl)oxy]phenyl}amino)pyrimidin-5-yl]ethynyl}phenyl)-β-alaninamide;
N-(3-{[4-({3-chloro-4-[(3-fluorobenzyl)oxy]phenyl}amino)pyrimidin-5-yl]ethynyl}phenyl)-2-(methylsulfonyl)acetamide;
N-[3-({4-[(4-benzylphenyl)amino]pyrimidin-5-yl}ethynyl)phenyl]-acetamide;
N-[3-({4-[(4-phenoxyphenyl)amino]pyrimidin-5-yl}ethynyl)phenyl]acetamide;
N-[3-({4-[(1-benzyl-1H-indazol-5-yl)amino]pyrimidin-5-yl}ethynyl)phenyl]acetamide;
1-benzyl-N-[5-(phenylethynyl)pyrimidin-4-yl]-1H-indol-5-amine;
5-[(6-amino-2-pyridinyl)ethynyl]-N-(3-chloro-4-{[(3-fluorophenyl)methyl]oxy}phenyl)-4-pyrimidinamine;
N-{6-[2-(4-{3-chloro-4-[(3-fluorobenzyl)oxy]anilino}-5-pyrimidinyl)ethynyl]-2-pyridinyl}acetamide;
2-chloro-N-{6-[(4-{3-chloro-4-[(3-fluorobenzyl)oxy]anilino}-5-pyrimidinyl)ethynyl]-2-pyridinyl}-2,2-difluoroacetamide;
N-{6-[(4-{3-chloro-4-[(3-fluorobenzyl)oxy]anilino}-5-pyrimidinyl)ethynyl]-2-pyridinyl}-4-(dimethylamino)butanamide;
methyl 4-({6-[(4-{3-chloro-4-[(3-fluorobenzyl)oxy]anilino}-5-pyrimidinyl)ethynyl]-2-pyridinyl}amino)-4-oxobutanoate;
N-{3-chloro-4-[(3-fluorobenzyl)oxy]phenyl}-5-[(6-{[2-(methylsulfonyl)ethyl]amino}-2-pyridinyl)ethynyl]-4-pyrimidinamine;
{6-[(4-{3-chloro-4-[(3-fluorobenzyl)oxy]anilino}-5-pyrimidinyl)ethynyl]-2-pyridinyl}methanol;
2-[({6-[(4{-3-chloro-4-[(3-fluorobenzyl)oxy]anilino}-5-pyrimidinyl)ethynyl]-2-pyridinyl}methyl)(methyl)amino]ethanol;
3-[({6-[(4-{3-chloro-4-[(3-fluorobenzyl)oxy]anilino}-5-pyrimidinyl)ethynyl]-2-pyridinyl}methyl)amino]propanenitrile;
N-{3-chloro-4-[(3-fluorobenzyl)oxy]phenyl}-5-{[6-({[2-(4-morpholinyl)ethyl]amino}methyl)-2-pyridinyl]ethynyl}-4-pyrimidinamine;
N-{2-[({6-[2-(4-{3-chloro-4-[(3-fluorobenzyl)oxy]anilino}-5-pyrimidinyl)ethynyl]-2-pyridinyl}methyl)amino]ethyl}acetamide;
N-{3-chloro-4[(3-fluorobenzyl)oxy]phenyl}-5-{[6-({[3-(1H-imidazol-1-yl)propyl]amino}methyl)-2-pyridinyl]ethynyl}-4-pyrimidinamine;
N-{3-chloro-4-[(3-fluorobenzyl)oxy]phenyl}-5-({6-[(methylamino)methyl]-2-pyridinyl}ethynyl)-4-pyrimidinamine;
N-{3-chloro-4-[(3-fluorobenzyl)oxy]phenyl}-5-{[6-(methoxymethyl)-2-pyridinyl]ethynyl}-4-pyrimidinamine;
N-{3-chloro-4-[(3-fluorobenzyl)oxy]phenyl}-5-{[2-(methylsulfanyl)-4-pyrimidinyl]ethynyl}-4-pyrimidinamine;
N-{3-chloro-4-[(3-fluorobenzyl)oxy]phenyl}-5-({6-[(dimethylamino)methyl]-2-pyridinyl}ethynyl)-4-pyrimidinamine;
N-benzyl-N-({6-[(4-{3-chloro-4-[(3-fluorobenzyl)oxy]anilino}-5-pyrimidinyl)ethynyl]-2-pyridinyl}methyl)amine;
N-{3-chloro-4-[(3-fluorobenzyl)oxy]phenyl}-5-[(6-{([(2-methoxyethyl)amino]methyl}-2-pyridinyl)ethynyl]-4-pyrimidinamine;
5-{[6-(aminomethyl)-2-pyridinyl]ethynyl}-N-{3-chloro-4-[(3-fluorobenzyl)oxy]phenyl}-4-pyrimidinamine;
N-({6-[(4-{3-chloro-4-[(3-fluorobenzyl)oxy]anilino}-5-pyrimidinyl)ethynyl]-2-pyridinyl}methyl)-N′-(2-cyanoethyl)urea;
N′-({6-[(4-{3-chloro-4-[(3-fluorobenzyl)oxy]anilino}-5-pyrimidinyl)ethynyl]-2-pyridinyl}methyl)-N-(2-hydroxyethyl)-N-methylurea;
N-({6-[(4-{3-chloro-4-[(3-fluorobenzyl)oxy]anilino}-5-pyrimidinyl)ethynyl]-2-pyridinyl}methyl)-N′-[2-(methylsulfonyl)ethyl]urea;
N-({6-[(4-{3-chloro-4-[(3-fluorobenzyl)oxy]anilino}-5-pyrimidinyl)ethynyl]-2-pyridinyl}methyl)-N′-[2-(4-morpholinyl)ethyl]urea;
N-({6-[(4-{3-chloro-4-[(3-fluorobenzyl)oxy]anilino}-5-pyrimidinyl)ethynyl]-2-pyridinyl}methyl)-N′-methylurea;
N-({6-[(4-{3-chloro-4-[(3-fluorobenzyl)oxy]anilino}-5-pyrimidinyl)ethynyl]-2-pyridinyl}methyl)-N′-(2-methoxyethyl)urea;
N-{3-chloro-4-[(3-fluorobenzyl)oxy]phenyl}-5-{[6-(1-piperidinylmethyl)-2-pyridinyl]ethynyl}-4-pyrimidinamine;
N-{3-chloro-4-[(3-fluorobenzyl)oxy]phenyl}-5-({6-[(4-methyl-1-piperazinyl)methyl]-2-pyridinyl}ethynyl)-4-pyrimidinamine;
N-{3-chloro-4-[(3-fluorobenzyl)oxy]phenyl}-5-{[6-(4-morpholinyl-methyl)-2-pyridinyl]ethynyl}-4-pyrimidinamine;
N-{3-chloro-4-[(3-fluorobenzyl)oxy]phenyl}-5-{[6-(1-pyrrolidinyl-methyl)-2-pyridinyl]ethynyl}-4-pyrimidinamine;
N-{3-chloro-4-[(3-fluorobenzyl)oxy]phenyl}-5-{[6-(1-piperazinylmethyl)-2-pyridinyl]ethynyl}-4-pyrimidinamine;
4-amino-2-{[4-({3-chloro-4-[(4-fluorobenzyl)oxy]phenyl}amino)pyrimidin-5-yl]ethynyl}pyrimidine-5-carbonitrile;
2-{[4-({3-chloro-4-[(3-fluorobenzyl)oxy]phenyl}amino)pyrimidin-5-yl]ethynyl}-4-{[2-(methylsulfonyl)ethyl]amino}pyrimidine-5-carbonitrile;
4-{[4-({3-chloro-4-[(3-fluorobenzyl)oxy]phenyl}amino)pyrimidin-5-yl]ethynyl}pyrimidin-2-amine; and
N-(6-{[4-({3-chloro-4-[(3-fluorobenzyl)oxy]phenyl}amino)pyrimidin-5-yl]ethynyl}pyridin-2-yl)-2,2,2-trifluoroacetamide;
   or a salt, solvate, or physiologically functional derivative thereof.

Typically, the salts of the present invention are pharmaceutically acceptable salts. Salts encompassed within the term “pharmaceutically acceptable salts” refer to non-toxic salts of the compounds of this invention. Salts of the compounds of the present invention may comprise acid addition salts derived from a nitrogen on a substituent in the compound of formula (I). Representative salts include the following salts: acetate, benzenesulfonate, benzoate, bicarbonate, bisulfate, bitartrate, borate, bromide, calcium edetate, camsylate, carbonate, chloride, clavulanate, citrate, dihydrochloride, edetate, edisylate, estolate, esylate, fumarate, gluceptate, gluconate, glutamate, glycollylarsanilate, hexylresorcinate, hydrabamine, hydrobromide, hydrochloride, hydroxynaphthoate, iodide, isethionate, lactate, lactobionate, laurate, malate, maleate, mandelate, mesylate, methylbromide, methylnitrate, methylsulfate, monopotassium maleate, mucate, napsylate, nitrate, N-methylglucamine, oxalate, pamoate (embonate), palmitate, pantothenate, phosphate/diphosphate, polygalacturonate, potassium, salicylate, sodium, stearate, subacetate, succinate, tannate, tartrate, teoclate, tosylate, triethiodide, trimethylammonium and valerate. Other salts, which are not pharmaceutically acceptable, may be useful in the preparation of compounds of this invention and these form a further aspect of the invention.

While it is possible that, for use in therapy, therapeutically effective amounts of a compound of formula (I), as well as salts, solvates and physiological functional derivatives thereof, may be administered as the raw chemical, it is possible to present the active ingredient as a pharmaceutical composition. Accordingly, the invention further provides pharmaceutical compositions, which include therapeutically effective amounts of compounds of the formula (I) and salts, solvates and physiological functional derivatives thereof, and one or more pharmaceutically acceptable carriers, diluents, or excipients. The compounds of the formula (I) and salts, solvates and physiological functional derivatives thereof, are as described above. The carrier(s), diluent(s) or excipient(s) must be acceptable in the sense of being compatible with the other ingredients of the formulation and not deleterious to the recipient thereof. In accordance with another aspect of the invention there is also provided a process for the preparation of a pharmaceutical formulation including admixing a compound of the formula (I), or salts, solvates and physiological functional derivatives thereof, with one or more pharmaceutically acceptable carriers, diluents or excipients.

Pharmaceutical formulations may be presented in unit dose forms containing a predetermined amount of active ingredient per unit dose. Such a unit may contain, for example, 0.5 mg to 1 g, preferably 1 mg to 700 mg, more preferably 5 mg to 100 mg of a compound of the formula (I), depending on the condition being treated, the route of administration and the age, weight and condition of the patient, or pharmaceutical formulations may be presented in unit dose forms containing a predetermined amount of active ingredient per unit dose. Preferred unit dosage formulations are those containing a daily dose or sub-dose, as herein above recited, or an appropriate fraction thereof, of an active ingredient. Furthermore, such pharmaceutical formulations may be prepared by any of the methods well known in the pharmacy art.

Pharmaceutical formulations may be adapted for administration by any appropriate route, for example by the oral (including buccal or sublingual), rectal, nasal, topical (including buccal, sublingual or transdermal), vaginal or parenteral (including subcutaneous, intramuscular, intravenous or intradermal) route. Such formulations may be prepared by any method known in the art of pharmacy, for example by bringing into association the active ingredient with the carrier(s) or excipient(s).

Pharmaceutical formulations adapted for oral administration may be presented as discrete units such as capsules or tablets; powders or granules; solutions or suspensions in aqueous or non-aqueous liquids; edible foams or whips; or oil-in-water liquid emulsions or water-in-oil liquid emulsions.

For instance, for oral administration in the form of a tablet or capsule, the active drug component can be combined with an oral, non-toxic pharmaceutically acceptable inert carrier such as ethanol, glycerol, water and the like. Powders are prepared by comminuting the compound to a suitable fine size and mixing with a similarly comminuted pharmaceutical carrier such as an edible carbohydrate, as, for example, starch or mannitol. Flavoring, preservative, dispersing and coloring agent can also be present.

Capsules are made by preparing a powder mixture, as described above, and filling formed gelatin sheaths. Glidants and lubricants such as colloidal silica, talc, magnesium stearate, calcium stearate or solid polyethylene glycol can be added to the powder mixture before the filling operation. A disintegrating or solubilizing agent such as agar-agar, calcium carbonate or sodium carbonate can also be added to improve the availability of the medicament when the capsule is ingested.

Moreover, when desired or necessary, suitable binders, lubricants, disintegrating agents and coloring agents can also be incorporated into the mixture. Suitable binders include starch, gelatin, natural sugars such as glucose or beta-lactose, corn sweeteners, natural and synthetic gums such as acacia, tragacanth or sodium alginate, carboxymethylcellulose, polyethylene glycol, waxes and the like. Lubricants used in these dosage forms include sodium oleate, sodium stearate, magnesium stearate, sodium benzoate, sodium acetate, sodium chloride and the like. Disintegrators include, without limitation, starch, methyl cellulose, agar, bentonite, xanthan gum and the like. Tablets are formulated, for example, by preparing a powder mixture, granulating or slugging, adding a lubricant and disintegrant and pressing into tablets. A powder mixture is prepared by mixing the compound, suitably comminuted, with a diluent or base as described above, and optionally, with a binder such as carboxymethylcellulose, an aliginate, gelatin, or polyvinyl pyrrolidone, a solution retardant such as paraffin, a resorption accelerator such as a quaternary salt and/or an absorption agent such as bentonite, kaolin or dicalcium phosphate. The powder mixture can be granulated by wetting with a binder such as syrup, starch paste, acadia mucilage or solutions of cellulosic or polymeric materials and forcing through a screen. As an alternative to granulating, the powder mixture can be run through the tablet machine and the result is imperfectly formed slugs broken into granules. The granules can be lubricated to prevent sticking to the tablet forming dies by means of the addition of stearic acid, a stearate salt, talc or mineral oil. The lubricated mixture is then compressed into tablets. The compounds of the present invention can also be combined with a free flowing inert carrier and compressed into tablets directly without going through the granulating or slugging steps. A clear or opaque protective coating consisting of a sealing coat of shellac, a coating of sugar or polymeric material and a polish coating of wax can be provided. Dyestuffs can be added to these coatings to distinguish different unit dosages.

Oral fluids such as solution, syrups and elixirs can be prepared in dosage unit form so that a given quantity contains a predetermined amount of the compound. Syrups can be prepared by dissolving the compound in a suitably flavored aqueous solution, while elixirs are prepared through the use of a non-toxic alcoholic vehicle. Suspensions can be formulated by dispersing the compound in a non-toxic vehicle. Solubilizers and emulsifiers such as ethoxylated isostearyl alcohols and polyoxy ethylene sorbitol ethers, preservatives, flavor additive such as peppermint oil or natural sweeteners or saccharin or other artificial sweeteners, and the like can also be added.

Where appropriate, dosage unit formulations for oral administration can be microencapsulated. The formulation can also be prepared to prolong or sustain the release as for example by coating or embedding particulate material in polymers, wax or the like.

The compounds of formula (I), and salts, solvates and physiological functional derivatives thereof, can also be administered in the form of liposome delivery systems, such as small unilamellar vesicles, large unilamellar vesicles and multilamellar vesicles. Liposomes can be formed from a variety of phospholipids, such as cholesterol, stearylamine or phosphatidylcholines.

The compounds of formula (I) and salts, solvates and physiological functional derivatives thereof may also be delivered by the use of monoclonal antibodies as individual carriers to which the compound molecules are coupled. The compounds may also be coupled with soluble polymers as targetable drug carriers. Such polymers can include polyvinylpyrrolidone, pyran copolymer, polyhydroxypropylmethacrylamide-phenol, polyhydroxyethylaspartamidephenol, or polyethyleneoxidepolylysine substituted with palmitoyl residues. Furthermore, the compounds may be coupled to a class of biodegradable polymers useful in achieving controlled release of a drug, for example, polylactic acid, polepsilon caprolactone, polyhydroxy butyric acid, polyorthoesters, polyacetals, polydihydropyrans, polycyanoacrylates and cross-linked or amphipathic block copolymers of hydrogels.

Pharmaceutical formulations adapted for transdermal administration may be presented as discrete patches intended to remain In intimate contact with the epidermis of the recipient for a prolonged period of time. For example, the active ingredient may be delivered from the patch by iontophoresis as generally described in Pharmaceutical Research, 3(6), 318 (1986).

Pharmaceutical formulations adapted for topical administration may be formulated as ointments, creams, suspensions, lotions, powders, solutions, pastes, gels, sprays, aerosols or oils.

For treatments of the eye or other external tissues, for example mouth and skin, the formulations are preferably applied as a topical ointment or cream. When formulated in an ointment, the active ingredient may be employed with either a paraffinic or a water-miscible ointment base. Alternatively, the active ingredient may be formulated in a cream with an oil-in-water cream base or a water-in-oil base.

Pharmaceutical formulations adapted for topical administrations to the eye include eye drops wherein the active ingredient is dissolved or suspended in a suitable carrier, especially an aqueous solvent.

Pharmaceutical formulations adapted for topical administration in the mouth include lozenges, pastilles and mouth washes.

Pharmaceutical formulations adapted for rectal administration may be presented as suppositories or as enemas.

Pharmaceutical formulations adapted for nasal administration wherein the carrier is a solid include a coarse powder having a particle size for example in the range 20 to 500 microns which is administered in the manner in which snuff is taken, i.e. by rapid inhalation through the nasal passage from a container of the powder held close up to the nose. Suitable formulations wherein the carrier is a liquid, for administration as a nasal spray or as nasal drops, include aqueous or oil solutions of the active ingredient.

Pharmaceutical formulations adapted for administration by inhalation include fine particle dusts or mists, which may be generated by means of various types of metered, dose pressurised aerosols, nebulizers or insufflators.

Pharmaceutical formulations adapted for vaginal administration may be presented as pessaries, tampons, creams, gels, pastes, foams or spray formulations.

Pharmaceutical formulations adapted for parenteral administration include aqueous and non-aqueous sterile injection solutions which may contain anti-oxidants, buffers, bacteriostats and solutes which render the formulation isotonic with the blood of the intended recipient; and aqueous and non-aqueous sterile suspensions which may include suspending agents and thickening agents. The formulations may be presented in unit-dose or multi-dose containers, for example sealed ampoules and vials, and may be stored in a freeze-dried (lyophilized) condition requiring only the addition of the sterile liquid carrier, for example water for injections, immediately prior to use. Extemporaneous injection solutions and suspensions may be prepared from sterile powders, granules and tablets.

It should be understood that in addition to the ingredients particularly mentioned above, the formulations may include other agents conventional in the art having regard to the type of formulation in question, for example those suitable for oral administration may include flavouring agents.

A therapeutically effective amount of a compound of the present invention will depend upon a number of factors including, for example, the age and weight of the human or other animal, the precise condition requiring treatment and its severity, the nature of the formulation, and the route of administration, and will ultimately be at the discretion of the attendant physician or veterinarian. However, an effective amount of a compound of formula (I) for the treatment of neoplastic growth, for example colon or breast carcinoma, will generally be in the range of 0.1 to 100 mg/kg body weight of recipient (mammal) per day and more usually in the range of 1 to 10 mg/kg body weight per day. Thus, for a 70 kg adult mammal, the actual amount per day would usually be from 70 to 700 mg and this amount may be given in a single dose per day or more usually in a number (such as two, three, four, five or six) of sub-doses per day such that the total daily dose is the same. An effective amount of a salt or solvate, or physiologically functional derivative thereof, may be determined as a proportion of the effective amount of the compound of formula (I) per se. It is envisaged that similar dosages would be appropriate for treatment of the other conditions referred to above.

The compounds of the present invention and their salts and solvates, and physiologically functional derivatives thereof, may be employed alone or in combination with other therapeutic agents for the treatment of the above-mentioned conditions. In particular, in anti-cancer therapy, combination with other chemotherapeutic, hormonal or antibody agent is envisaged as well as combination with surgical therapy and radiotherapy. Combination therapies according to the present invention thus comprise the administration of at least one compound of formula (I) or a pharmaceutically acceptable salt or solvate thereof, or a physiologically functional derivative thereof, and the use of at least one other cancer treatment method. Preferably, combination therapies according to the present invention comprise the administration of at least one compound of formula (I) or a pharmaceutically acceptable salt or solvate thereof, or a physiologically functional derivative thereof, and at least one other pharmaceutically active agent, preferably an anti-neoplastic agent. The compound(s) of formula (I) and the other pharmaceutically active agent(s) may be administered together or separately and, when administered separately this may occur simultaneously or sequentially in any order. The amounts of the compound(s) of formula (I) and the other pharmaceutically active agent(s) and the relative timings of administration will be selected in order to achieve the desired combined therapeutic effect.

The compounds of the Formula (I) or salts, solvates, or physiologically functional derivatives thereof and at least one additional cancer treatment therapy may be employed in combination concomitantly or sequentially in any therapeutically appropriate combination with such other anti-cancer therapies. In one embodiment, the other anti-cancer therapy is at least one additional chemotherapeutic therapy including administration of at least one anti-neoplastic agent. The administration in combination of a compound of formula (I) or salts, solvates, or physiologically functional derivatives thereof with other anti-neoplastic agents may be in combination in accordance with the invention by administration concomitantly in (1) a unitary pharmaceutical composition including both compounds, or (2) separate pharmaceutical compositions each including one of the compounds. Alternatively, the combination may be administered separately in a sequential manner wherein one anti-neoplastic agent is administered first and the other second or vice versa. Such sequential administration may be close in time or remote in time.

Anti-neoplastic agents may induce anti-neoplastic effects in a cell-cycle specific manner, i.e., are phase specific and act at a specific phase of the cell cycle, or bind DNA and act in a non cell-cycle specific manner, i.e., are non-cell cycle specific and operate by other mechanisms.

Anti-neoplastic agents useful in combination with the compounds and salts, solvates or physiologically functional derivatives thereof of formula I include, but are not limited to, the following:

(1) cell cycle specific anti-neoplastic agents including, but not limited to, diterpenoids such as paclitaxel and its analog docetaxel; vinca alkaloids such as vinblastine, vincristine, vindesine, and vinorelbine; epipodophyllotoxins such as etoposide and teniposide; fluoropyrimidines such as 5-fluorouracil and fluorodeoxyuridine; antimetabolites such as allopurinol, fludurabine, methotrexate, cladrabine, cytarabine, mercaptopurine and thioguanine; and camptothecins such as 9-amino camptothecin, irinotecan, CPT-11 and the various optical forms of 7-(4-methylpiperazino-methylene)-10,11-ethylenedioxy-20-camptothecin;

(2) cytotoxic chemotherapeutic agents including, but not limited to, alkylating agents such as melphalan, chlorambucil, cyclophosphamide, mechlorethamine, hexamethylmelamine, busulfan, carmustine, lomustine, and dacarbazine; anti-tumour antibiotics such as doxorubicin, daunomycin, epirubicin, idarubicin, mitomycin-C, dacttinomycin and mithramycin; and platinum coordination complexes such as cisplatin, carboplatin, and oxaliplatin; and

(3) other chemotherapeutic agents including, but not limited to, anti-estrogens such as tamoxifen, toremifene, raloxifene, droloxifene and iodoxyfene; progestrogens such as megestrol acetate; aromatase inhibitors such as anastrozole, letrazole, vorazole, and exemestane; antiandrogens such as flutamide, nilutamide, bicalutamide, and cyproterone acetate; LHRH agonists and antagagonists such as goserelin acetate and luprolide, testosterone 5α-dihydroreductase inhibitors such as finasteride; metalloproteinase inhibitors such as marimastat; antiprogestogens; urokinase plasminogen activator receptor function inhibitors; cyclooxygenase type 2 (COX-2) inhibitors such as celecoxib; angiogenic inhibiting agents such as VEGFR inhibitors and TIE-2 inhibitors; growth factor function inhibitors such as inhibitors of the functions of hepatocyte growth factor; platelet derived growth factor receptor (PDGFr), vascular endothelial growth factor receptor (VEGFR) and TIE-2; and other protein kinase inhibitors such as c-Raf, b-Raf, and cyclin dependent inhibitors such as CDK2 and CDK4 inhibitors.

The compounds of formula (I) and salts, solvates and physiological functional derivatives thereof, are believed to have anticancer activity as a result of inhibition of one or more erbB family protein kinase and its effect on selected cell lines whose growth is dependent on erbB family protein kinase activity.

The present invention thus also provides compounds of formula (I) and pharmaceutically acceptable salts or solvates thereof, or physiologically functional derivatives thereof, for use in medical therapy, and particularly in the treatment of disorders mediated by inappropriate activity of one or more erbB family kinase.

The inappropriate erbB family activity referred to herein is any erbB kinase activity that deviates from the normal erbB family kinase activity expected in a particular mammalian subject. The inappropriate activity may arise from one or more of EGFR (erbB-1), erbB-2, or erbB-4. Inappropriate erbB family kinase activity may take the form of, for instance, an abnormal increase in activity, or an aberration in the timing and or control of erbB family kinase activity. Such inappropriate activity may result then, for example, from overexpression or mutation of the protein kinase or ligand leading to inappropriate or uncontrolled activation of the receptor. Furthermore, it is also understood that unwanted erbB family kinase activity may reside in an abnormal source, such as a malignancy. That is, the level of erbB family activity does not have to be abnormal to be considered inappropriate rather the activity derives from an abnormal source.

The present invention is directed to methods of regulating, modulating, or inhibiting one or more erbB family kinase for the prevention and/or treatment of disorders related to unregulated erbB family kinase activity. In particular, the compounds of the present invention can also be used in the treatment of certain forms of cancer. Furthermore, the compounds of the present invention can be used to provide additive or synergistic effects with certain existing cancer chemotherapies and radiation, and/or be used to restore effectiveness of certain existing cancer chemotherapies and radiation.

A further aspect of the invention provides a method of treatment of a mammal suffering from a disorder mediated by one or more inappropriate erbB family kinase activity, including susceptible malignancies, which includes administering to said subject an effective amount of a compound of formula (I) or a pharmaceutically acceptable salt, solvate, or a physiologically functional derivative thereof. In a preferred embodiment, the disorder is cancer.

A further aspect of the invention provides a method of treatment of a mammal suffering from cancer, which includes administering to said subject an effective amount of a compound of formula (I) or a pharmaceutically acceptable salt or solvate thereof, or a physiologically functional derivative thereof.

A further aspect of the present invention provides the use of a compound of formula (I), or a pharmaceutically acceptable salt or solvate thereof, or a physiologically functional derivative thereof, in the preparation of a medicament for the treatment of a disorder characterized by inappropriate activity of one or more erbB family kinase. In a preferred embodiment, the disorder is cancer.

A further aspect of the present invention provides the use of a compound of formula (I), or a pharmaceutically acceptable salt or solvate thereof, or a physiologically functional derivative thereof, in the preparation of a medicament for the treatment of cancer and malignant tumours.

The mammal requiring treatment with a compound of the present invention is typically a human being.

In another embodiment, therapeutically effective amounts of the compounds of formula (I) or salts, solvates or physiologically derived derivatives thereof and agents which inhibit growth factor receptor function may be administered in combination to a mammal for treatment of a disorder mediated by inappropriate activity of one or more erbB family kinase, for instance in the treatment of cancer. Such growth factor receptors include, for example, PDGFR, VEGFR, TIE-2, as well as erbB family kinase inhibitors other than those described herein. Growth factor receptors and agents that inhibit growth factor receptor function are described, for instance, in Kath, John C., Exp. Opin. Ther. Patents (2000) 10(6):803-818 and in Shawver et al DDT Vol 2, No. 2 Feb. 1997.

The compounds of the Formula (I) or salts, solvates, or physiologically functional derivatives thereof and the agent for inhibiting growth factor receptor function may be employed in combination concomitantly or sequentially in any therapeutically appropriate combination. The combination may be employed in combination in accordance with the invention by administration concomitantly in (1) a unitary pharmaceutical composition including both compounds, or (2) separate pharmaceutical compositions each including one of the compounds. Alternatively, the combination may be administered separately in a sequential manner wherein one is administered first and the other second or vice versa. Such sequential administration may be close in time or remote in time.

The compounds of this invention may be made by a variety of methods, including standard chemistry. Any previously defined variable will continue to have the previously defined meaning unless otherwise indicated. Illustrative general synthetic methods are set out below and then specific compounds of the invention are prepared in the working Examples.

Compounds of general Formula (I) may be prepared by methods known in the art of organic synthesis as set forth in part by the following synthesis schemes. In all of the schemes described below, it is well understood that protecting groups for sensitive or reactive groups are employed where necessary in accordance with general principles of chemistry. Protecting groups are manipulated according to standard methods of organic synthesis (T. W. Green and P. G. M. Wuts (1991) Protecting Groups in Organic Synthesis, John Wiley & Sons). These groups are removed at a convenient stage of the compound synthesis using methods that are readily apparent to those skilled in the art. The selection of processes as well as the reaction conditions and order of their execution shall be consistent with the preparation of compounds of Formula (I). Those skilled in the art will recognize if a stereocenter exists in compounds of Formula (I). Accordingly, the present invention includes both possible stereoisomers and includes not only racemic compounds but the individual enantiomers as well. When a compound is desired as a single enantiomer, it may be obtained by stereospecific synthesis or by resolution of the final product or any convenient intermediate. Resolution of the final product, an intermediate, or a starting material may be effected by any suitable method known in the art. See, for example, Stereochemistry of Organic Compounds by E. L. Eliel, S. H. Wilen, and L. N. Mander (Wiley-Interscience, 1994).

A general method for preparing compounds of the general formula (I) wherein A is an alkynylene group involves the reaction of a compound of general formula (A) with a compound of general formula (B). Alternatively, a general method for preparing compounds of the general formula (I) involves the reaction of a compound of general formula (C) with a compound of general formula (D). Formula (A), formula (B), formula (C), and formula (D) are depicted in Scheme 1.

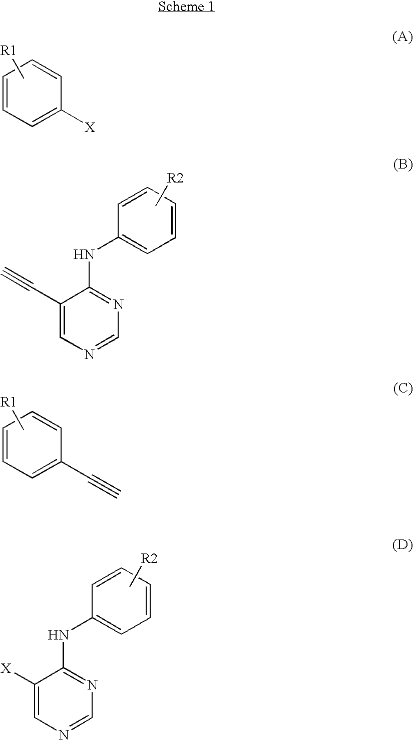

X is halogen, triflate, tosylate, or other common leaving group in such metal-mediated reactions. Groups R¹ and R², recited In Schemes 1 to 6, are as defined above. The general method can be readily carried out by mixing a compound of general formula (A) with a compound of general formula (B) in a suitable solvent, in the presence of an amine base, a paladium catalyst such as PdCl₂(PPh₃)₂, a copper salt such as copper (I) iodide and heating the reaction mixture to about 35-150° C. Typically the solvent is THF or DMF, and the amine base can be, for example, triethyl amine. Alternatively, the general method can be readily carried out by mixing a compound of general formula (C) with a compound of general formula (D) in a suitable solvent, in the presence of an amine base, a paladium catalyst such as PdCl₂(PPh₃)₂, a copper salt such as copper (I) iodide and heating the reaction mixture to about 35-150° C. Typically the solvent is THF or DMF, and the amine base can be, for example, triethyl amine. This reaction is commonly known as a Sonogashira reaction or Sonogashira coupling.

A general method for preparing compounds of the general formula (I) wherein A is an alkenylene group involves the reaction of a compound of general formula (A) with a compound of general formula (E). Alternatively, a general method for preparing compounds of the general formula (II) involves the reaction of a compound of general formula (F) with a compound of general formula (D). Formula (A), formula (E), formula (F), and formula (D) are depicted in Scheme 2.

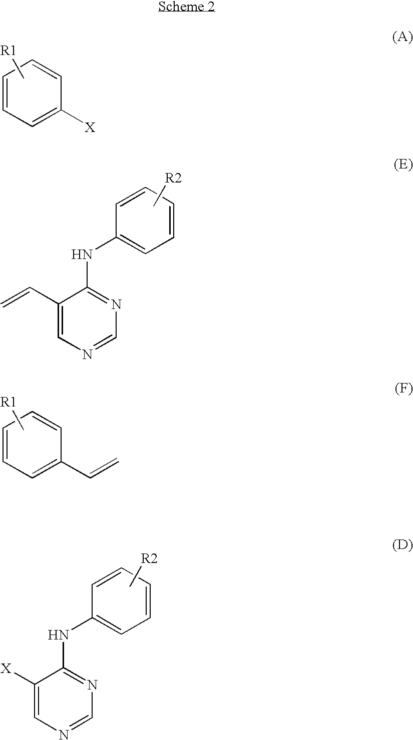

X is halogen, triflate, tosylate, or other common leaving group in such metal-mediated reactions. Groups R¹ and R² are as defined above. The general method can be readily carried out by mixing a compound of general formula (A) with a compound of general formula (E) in a suitable solvent, in the presence of a palladium catalyst such as Pd(OAc)₂, a phosphine source such as tri-o-tolylphosphine, and heating the reaction mixture to about 35-150° C. Typically the solvent is THF or DMF. Alternatively, the general method can be readily carried out by mixing a compound of general formula (F) with a compound of general formula (D) in a suitable solvent, in the presence of a palladium catalyst such as Pd(OAc)₂, a phosphine source such as tri-o-tolylphosphine, and heating the reaction mixture to about 35-150° C. Typically the solvent is THF or DMF. This reaction is commonly known as a Heck reaction or Heck coupling.

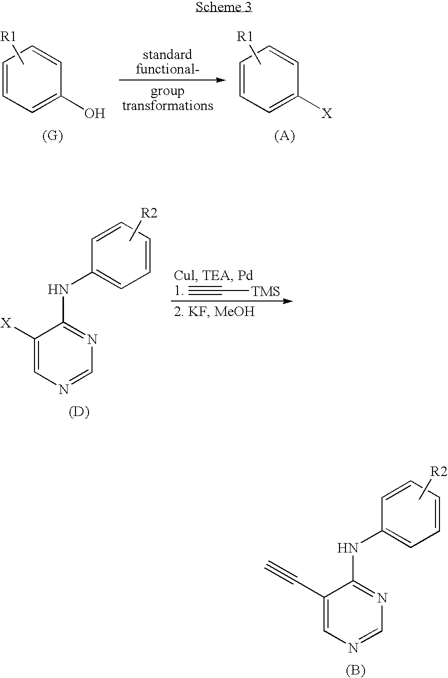

As shown in Scheme 3, compounds of general formula (A) may be commercially available, are known in the literature, or may be conveniently prepared by reacting a compound of general formula (G) with reagents standard to one skilled in the arts to give the halogen, triflate, or tosyl leaving groups of compounds of formula (A). Compounds of general formula (B) may be conveniently prepared by reacting a compound of general formula (D) with TMS-acetylene, a copper (I) salt, an amine base, and palladium such as PdCl₂(PPh₃)₂ followed by treatment with a fluoride source such as TBAF or KF to give compounds of formula (B).

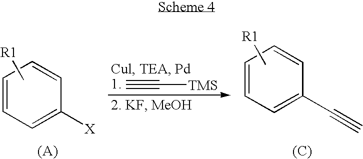

As shown in Scheme 4, compounds of general formula (C) may be commercially available, are known in the literature, or may be conveniently prepared by reacting a compound of general formula (A) with TMS-acetylene, a copper (I) salt, an amine base, and palladium such as PdCl₂(PPh₃)₂ followed by treatment with a fluoride source such as TBAF or KF to give compounds of formula (C).

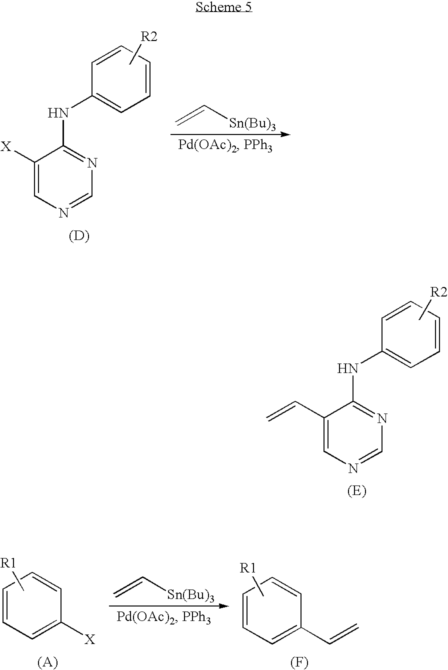

As shown in Scheme 5, compounds of general formula (E) can be readily prepared by mixing a compound of general formula (D) with vinyltributylstannane in a suitable solvent, in the presence of a palladium catalyst such as Pd(OAc)₂, a phosphine source such as tri-o-tolylphosphine, and heating the reaction mixture to about 35-150° C. to give compounds of general formula (E). Compounds of general formula (F) may be commercially available, are known in the literature, or may be conveniently prepared by reacting a compound of general formula (A) with vinyltributylstannane in a suitable solvent, in the presence of a palladium catalyst such as Pd(OAc)₂, a phosphine source such as tri-o-tolylphosphine, and heating the reaction mixture to about 35-150° C. to give compounds of general formula (F).

As shown in Scheme 6, compounds of general formula (D) may be conveniently prepared by reacting a compound of general formula (H) with a compound of general formula (J) in an alcohol solvent, optionally with a base such as potasium carbonate, optionally with an acid such as hydrochloric acid, optionally with heating from 35-150° C. to give compounds of general formula (D).

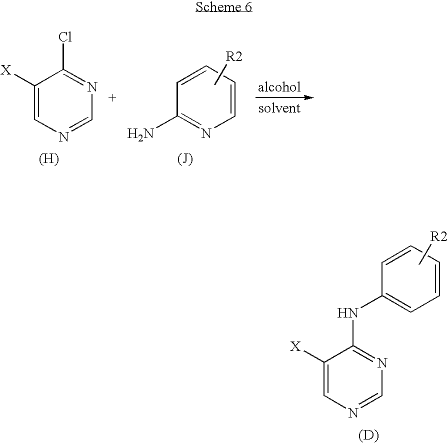

Compounds of general formula (H) may be commercially available and are known in the literature. Compounds of general formula (J) may be commercially available and are known in the literature, or may be prepared by known chemistry to those skilled in the arts.

Certain embodiments of the present invention will now be illustrated by way of example only. The physical data given for the compounds exemplified is consistent with the assigned structure of those compounds.

### EXAMPLES

As used herein the symbols and conventions used in these processes, schemes and examples are consistent with those used in the contemporary scientific literature, for example, the Journal of the American Chemical Society or the Journal of Biological Chemistry. Standard single-letter or three-letter abbreviations are generally used to designate amino acid residues, which are assumed to be in the L-configuration unless otherwise noted. Unless otherwise noted, all starting materials were obtained from commercial suppliers and used without further purification. Specifically, the following abbreviations may be used in the examples and throughout the specification:

g (grams); mg (milligrams);

L (liters); mL (milliliters);

μL (microliters); psi (pounds per square inch);

M (molar); mM (millimolar);

i.v. (intravenous); Hz (Hertz);

MHz (megahertz); mol (moles);

mmol (millimoles); RT (room temperature);

min (minutes); h (hours);

mp (melting point); TLC (thin layer chromatography);

Tr (retention time); RP (reverse phase);

MeOH (methanol); I-PrOH (isopropanol);

TEA (triethylamine); TFA (trifluoroacetic acid);

TFAA (trifluoroacetic anhydride); THF (tetrahydrofuran);

DMSO (dimethylsulfoxide); EtOAc or EA (ethyl acetate);

DME (1,2-dimethoxyethane); DCM (dichloromethane);

DCE (dichloroethane); DMF (N,N-dimethylformamide);

DMPU (N,N′-dimethylpropyleneurea);

(CDI (1,1-carbonyldiimidazole); IBCF (isobutyl chloroformate);

HOAc (acetic acid);

HOSu (N-hydroxysuccinimide); HOBT (1-hydroxybenzotriazole);

mCPBA (meta-chloroperbenzoic acid;

EDC (ethylcarbodiimide hydrochloride);

BOC (tert-butyloxycarbonyl); FMOC (9-fluorenylmethoxycarbonyl);

DCC (dicyclohexylcarbodiimide); CBZ (benzyloxycarbonyl);

Ac (acetyl); atm (atmosphere);

TMSE (2-(trimethylsilyl)ethyl); TMS (trimethylsilyl);

TIPS (triisopropylsilyl); TBS (t-butyldimethylsilyl);

DMAP (4-dimethylaminopyridine); Me (methyl);

HPLC (high pressure liquid chromatography);

BOP (bis(2-oxo-3-oxazolidinyl)phosphinic chloride);

TBAF (tetra-n-butylammonium fluoride);

Et (ethyl); tBu (tert-butyl);

HOSA (hydroxylamine sulfonic acid); DEAD (diethylazodicarboxylate);

DIEA (diisopropylethylamine).

All references to ether are to diethyl ether; brine refers to a saturated aqueous solution of NaCl. Unless otherwise indicated, all temperatures are expressed in ° C. (degrees Centigrade). All reactions conducted under an inert atmosphere at room temperature unless otherwise noted.

¹H NMR spectra were recorded on a Varian VXR-300, a Varian Unity-300, a Varian Unity-400 instrument, or a General Electric QE-300. Chemical shifts are expressed in parts per million (ppm, δ units)) relative to Me₄Si. Coupling constants are in units of hertz (Hz). Splitting patterns describe apparent multiplicities and are designated as s (singlet), d (doublet), t (triplet), q (quartet), m (multiplet), br (broad).

Low-resolution mass spectra (MS) were recorded via LCMS on a Micromass ZQ, ZMD, or QuattroMicro spectrometer; high resolution MS were obtained using a JOEL SX-102A spectrometer. All mass spectra were taken under electrospray ionization (ESI), chemical ionization (CI), electron impact (EI), atmospheric pressure chemical ionization (APCI) or by fast atom bombardment (FAB) methods. Infrared (IR) spectra were obtained on a Nicolet 510 FT-IR spectrometer using a 1-mm NaCl cell. All reactions were monitored by thin-layer chromatography on 0.25 mm E. Merck silica gel plates (60F-254), visualized with UV light, 5% ethanolic phosphomolybdic acid or p-anisaldehyde solution. Flash column chromatography was performed on silica gel (230-400 mesh, Merck). Optical rotations were obtained using a Perkin Elmer Model 241 Polarimeter. Melting points were determined using a Mel-Temp II apparatus and are uncorrected.

### Example 1

### 2-Benzyl-N-{5-[(E)-2-phenylethenyl]pyrimidin-4-yl}-1H-benzimidazol-5-amine

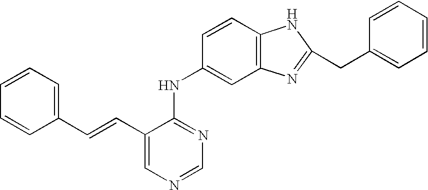

a) A mixture of 2-benzyl-N-(5-vinylpyrimidin-4-yl)-1H-benzimidazol-5-amine (100 mg, 0.30 mmol), phenyl iodide (61 mg, 0.30 mmol), palladium(II) acetate (7 mg), triethylamine (30 mg, 0.30 mmol), and tri-o-tolylphosphine (9 mg, 0.03 mmol) in dimethylformamide (1 mL) was heated at 100° C. for 5 h. The cooled reaction mixture was diluted with water and the resulting precipitate was collected by filtering, dried and chromatographed on silica gel eluting with an ethyl acetate to ethyl acetate:methanol 19:1 gradient to give a tan solid (10 mg, 8%). ¹H NMR (400 MHz, DMSO-d₆) δ 4.16 (s, 2H), 7.22-7.51 (m, 12H), 7.69 (d, J=8 Hz, 2H), 7.77 (s, 1H), 8.42 (s, 1H), 8.59 (s, 1H), 9.00 (s, 1H), 12.26 (brs, 1H); ESIMS: 404 (M+H)⁺

b) 2-Benzyl-N-(5-vinylpyrimidin-4-yl)-1H-benzimidazol-5-amine A mixture of 2-benzyl-N-(5-iodopyrimidin-4-yl)-1H-benzimidazol-5-amine (2.0 g, 4.7 mmol), vinyltributylstannane (1.8 mL, 6.1 mmol), tetrabutylammonium chloride (1.3 g, 4.7 mmol), palladium acetate (50 mg) and palladium(II) chloride (42 mg) in dimethylformamide (10 mL) was heated at 55° C. for 6 h. The reaction was cooled, diluted with ethyl acetate (200 mL) and water (200 mL) containing potassium fluoride (2.5 g), shaken well and filtered through celite. The ethyl acetate layer was separated, the aqueous extracted with additional ethyl acetate and the combined organic layers were dried, concentrated and chromatographed on silica gel eluting with an ethyl acetate to 9:1 ethyl acetate:methanol gradient to give a straw-colored solid (1.0 g, 67%). ¹H NMR (400 MHz, DMSO-d₆) δ 4.13 (s, 2H), 5.41 (d, J=12 Hz, 1H), 5.83 (d, J=17 Hz, 1H), 6.97-7.04 (m, 1H), 7.20-7.31 (m, 6H), 7.38 (d, J=8 Hz, 1H), 7.76 (s, 1H), 8.40 (s, 2H), 8.78 (s, 1H), 12.19 (brs, 1H); ESIMS: 328 (M+H)⁺

c) 2-Benzyl-N-(5-iodopyrimidin-4-yl)-1H-benzimidazol-5-amine A mixture of 4-chloro-5-iodopyrimidine (5.0 g, 21 mmol) and 2-benzyl-1H-benzimidazol-5-amine (4.7 g, 21 mmol) in absolute ethanol (250 mL) was refluxed for 16 h. The mixture was concentrated and partitioned between water containing hydrochloric acid (to pH 1) and ethyl acetate. The ethyl acetate layer was discarded. The aqueous layer was made basic by adding 50% sodium hydroxide solution (to pH 12) and extracted with ethyl acetate. The organic layer was dried and concentrated to an amber foam (6.2 g, 73%). The crude material was carried on to the next step. ESIMS: 429 (M+H)⁺

d) 4-Chloro-5-Iodopyrimidine To a stirred solution containing 7.7 mL (99 mmol) of DMF and 150 mL of dichloroethane at 0° C. was added 12.7 mL (144.6 mmol) of oxalyl chloride slowly to control vigorous gas evolution. After the evolution of gas had ceased, 10.0 g of iodopyrimidone was added and the reaction mixture was heated at reflux for 3 h, then cooled to room temperature and partitioned between water and dichloromethane. The organic layers were dried over MgSO₄ and the solvent was removed under reduced pressure to give 9.6 g (88%) of the title compound. ¹H-NMR (300 MHz, CDCl₃) δ 8.89 (s, 1H) and 8.98 (s, 1H); ESIMS: 241.1 (M+H)⁺

e) 5-Iodopyrimidin-4(3H)-one To a stirred solution containing 20.2 g (0.21 mol) of pyrimidin-4(3H)-one and 170 mL of water was added 10.9 g (0.27 mol) of sodium hydroxide, followed by 53.3 g (0.21 mol) of iodide. The reaction mixture was heated at 85° C. for 16 h, then cooled to room temperature and filtered. The filter cake was washed with water, collected, and dried under reduced pressure to give 29.7 g (64%) of the title compound. ¹H-NMR (300 MHz, DMSO-d₆) δ 8.17 (s, 1H), 8.43 (s, 1H), and 12.92 (brs, 1H); ESIMS: 223.1 (M+H)⁺

### Example 2

### 2-Benzyl-N-{5-[(E)-2-thien-3-ylethenyl]pyrimidin-4-yl}-1H-benzimidazol-5-amine

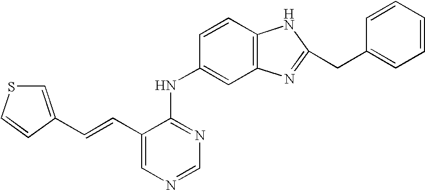

In a similar manner as described in Example 1a, from 3-iodothiophene (126 mg, 0.5 mmol) was obtained the title compound as a tan solid (7 mg, 3%). ¹H NMR (400 MHz, DMSO-d₆) δ4.15 (s, 2H), 7.21-7.32 (m, 9H), 7.42 (d, J=8 Hz, 1H), 7.56-7.60 (m, 4H), 8.39 (s, 1H), 8.51 (s, 1H), 8.91 (s, 1H); ESIMS: 410 (M+H)⁺

### Example 3

### 2-Benzyl-N-{5-[(E)-2-(1H-pyrazol-4-yl)ethenyl]pyrimidin-4-yl}-1H-benzimidazol-5-amine

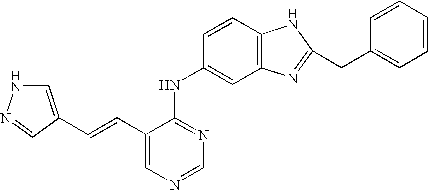

In a similar manner as described in Example 1a, from 1-acetyl-4-iodo-1H-pyrazole (71 mg, 0.3 mmol) was obtained the title compound as a tan solid (5 mg, 4%). ¹H NMR (400 MHz, DMSO-d₆) δ 4.14 (s, 2H), 7.05 (d, J=16 Hz, 1H), 7.12 (d, J=16 Hz, 1H), 7.20-7.31 (m, 7H), 7.41 (d, J=8 Hz, 1H), 7.76 (s, 1H), 7.87 (br s, 2H), 8.36 (s, 1H), 8.45 (s, 1H), 8.78 (s, 1H), 12.26 (br s, 1H); ESIMS: 394 (M+H)⁺

### Example 4

### 3-((E)-2-{4-[(2-Benzyl-1H-benzimidazol-5-yl)amino]pyrimidin-5-yl}ethenyl)-N-methylbenzamide

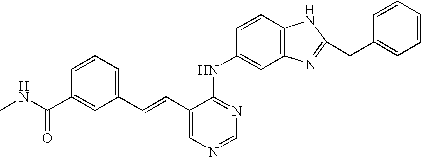

In a similar manner as described in Example 1a, from 3-iodo-N-methylbenzamide (78 mg, 0.3 mmol) was obtained the title compound as a tan solid (20 mg, 14%). ¹H NMR (400 MHz, DMSO-d₆) δ 2.78 (d, J=5 Hz, 3H), 4.15 (s, 2H), 7.20-7.32 (m, 8H), 7.42-7.55 (m, 3H), 7.70-7.81 (m, 3H), 8.11 (s, 1H), 8.42 (s, 1H), 8.46 (d, J=5 Hz, 1H), 8.58 (s, 1H), 9.07 (s, 1H); ESIMS: 461 (M+H)⁺

### Example 5

### 2-Benzyl-N-{5-[(E)-2-thien-3-ylethenyl]pyrimidin-4-yl}-1,3-benzothiazol-5-amine

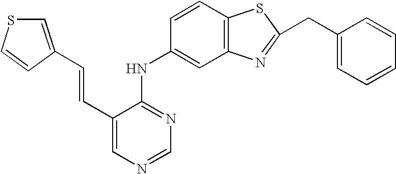

a) In a similar manner as described in Example 1a, from 2-benzyl-N-(5-vinylpyrimidin-4-yl)-1,3-benzothiazol-5-amine (210 mg, 0.6 mmol) and 3-Iodothiophene (126 mg, 0.6 mmol) was obtained the title compound as an amber oil (37 mg, 14%). ¹H NMR (400 MHz, DMSO-d₆) δ 4.44 (s, 2H), 7.32-7.39 (m, 4H), 7.52-7.61 (m, 7H), 7.92 (d, J=9 Hz, 1H), 8.32 (d, J=2 Hz, 1H), 8.50 (s, 1H), 8.59 (s, 1H), 9.08 (s, 1H); ESIMS: 427 (M+H)⁺

b) 2-Benzyl-N-(5-vinylpyrimidin-4-yl)-1,3-benzothiazol-5-amine In a similar manner as described in Example 1b, 2-benzyl-N-(5-iodopyrimidin-4-yl)-1,3-benzothiazol-5-amine (0.71 g, 1.6 mmol) gave the title compound as a tan solid (212 mg, 38%). ¹H NMR (400 MHz, DMSO-d₆) δ4.43 (s, 2H), 5.45 (d, J=12 Hz, 1H), 5.87 (d, J=17 Hz, 1H), 6.99-7.06 (m, 1H), 7.26-7.39 (m, 5H), 7.61 (dd, J=2, 9 Hz, 1H), 7.88 (d, J=9 Hz, 1H), 8.33 (d, J=2 Hz, 1H), 8.48 (s, 1H), 8.52 (s, 1H), 8.98 (s, 1H); ESIMS: 345 (M+H)⁺

c) 2-Benzyl-N-(5-iodopyrimidin-4-yl)-1,3-benzothiazol-5-amine Hydrochloride 2-Benzyl-1,3-benzothiazol-5-amine dihydrochloride (5.0 g, 18 mmol) was partitioned between saturated sodium bicarbonate solution (50 mL) and dichloromethane (100 mL). The organic layer was dried and concentrated to a brown solid. A mixture of this sample of 2-benzyl-1,3-benzothiazol-5-amine (3.8 g, 15.8 mmol) and 4-chloro-5-iodopyrimidine (3.8 g, 15.8 mmol) in ethanol (200 mL) was refluxed under nitrogen for 3 h, cooled to rt and the resulting tan precipitate was collected by filtering and dried (5.3 g, 71%). ¹H NMR (400 MHz, DMSO-d₆) δ 4.46 (s, 2H), 7.27-7.39 (m, 5H), 7.48 (d, J=8 Hz, 1H), 7.99 (d, J=8 Hz, 1H), 8.10 (d, J=2 Hz, 1H), 8.66 (s, 1H), 8.83 (d, J=7 Hz, 1H), 9.66 (s, 1H); ESIMS: 445 (M+H)⁺

d) 2-Benzyl-1,3-benzothiazol-5-amine Dihydrochloride A mixture of 2-benzyl-5-nitro-1,3-benzothiazole (15.0 g, 55 mmol) and tin(II) chloride dihydrate (45.0 g, 0.2 mmol) in absolute ethanol (500 mL) was refluxed for 3 h. The mixture was cooled to rt, basified by slow addition of saturated sodium bicarbonate solution (600 mL) and extracted with ethyl acetate. The organic layer was dried, concentrated, redissolved in ethyl acetate (100 mL) and 4N hydrochloric acid in dioxane (15 mL) was slowly added. The resulting gray solid was collected and recrystallized from ethanol to give a gray solid (17.1 g, 81%). ¹H NMR (400 MHz, DMSO-d₆) δ 4.48 (s, 2H), 7.27-7.40 (m, 6H), 7.95 (d, J=2 Hz, 1H), 8.11 (d, J=9 Hz, 1H), 10.44 (br s, 3H); APIMS: 241 (M+H)⁺

e) 2-Benzyl-5-nitro-1,3-benzothiazole Phenylacetyl chloride (28 mL, 0.21 mmol) was added dropwise to a stirred solution of 2-amino-4-nitrobenzenethiol (35.5 g, 0.21 mol) in 1-methyl-2-pyrrolidinone (150 mL) at rt under a nitrogen atmosphere. The resulting yellow solution was heated at 100° C. for 1 h, cooled to rt, diluted with water (450 mL), adjusted to pH 9 by adding 28% ammonium hydroxide and the resulting yellow precipitate was collected by filtering, washed with water and dried (55.4 g, 98%). ¹H NMR (400 MHz, DMSO-d₆) δ 4.53 (s, 2H), 7.26-7.41 (m, 5H), 8.21 (dd, J=2, 9 Hz, 1H), 8.28 (d, J=9 Hz, 1H), 8.71 (d, J=2 Hz, 1H); ESIMS: 269 (M−H)⁻

### Example 6

### 1-Benzyl-N-{5-[(E)-2-pyridin-3-ylethenyl]pyrimidin-4-yl}-1H-indazol-5-amine

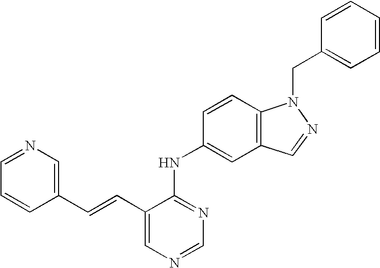

a) In a similar manner as described in Example 1a, from 3-iodopyridine (62 mg, 0.3 mmol) and 1-benzyl-N-(5-vinylpyrimidin-4-yl)-1H-indazol-5-amine (98 mg, 0.3 mmol) was obtained the title compound as an amber glass (27 mg, 22%). ¹H NMR (400 MHz, DMSO-d₆) δ 5.63 (s, 2H), 7.20-7.31 (m, 6H), 7.41-7.67 (m, 4H), 7.94 (s, 1H), 8.07 (s, 1H), 8.08 (d, J=8 Hz, 1H), 8.43 (s, 1H), 8.47 (d, J=4 Hz, 1H), 8.61 (s, 1H), 8.84 (s, 1H), 9.06 (s, 1H); ESIMS: 405 (M+H)⁺

b) 1-Benzyl-N-(5-vinylpyrimidin-4-yl)-1H-indazol-5-amine In a similar manner as described in Example 1b, 1-benzyl-N-(5-iodopyrimidin-4-yl)-1H-indazol-5-amine (2.0 g, 4.7 mmol) gave the title compound as a solid (1.0 g, 67%). ¹H NMR (400 MHz, DMSO-d₆) δ 5.42 (d, J=12 Hz, 1H), 5.62 (s, 2H), 5.85 (d, J=17 Hz, 1H), 6.96-7.03 (m, 1H), 7.19-7.30 (m, 5H), 7.48 (dd, J=2, 9 Hz, 1H), 7.61 (d, J=9 Hz, 1H), 7.95 (d, J=2 Hz, 1H), 8.05 (s, 1H), 8.42 (s, 2H), 8.86 (s, 1H); ESIMS: 328 (M+H)⁺

c) 1-Benzyl-N-(5-iodopyrimidin-4-yl)-1H-indazol-5-amine In a similar manner as described in Example 1c, from 1-benzyl-1H-indazol-5-amine (3.7 g, 16.6 mmol) and 4-chloro-5-iodopyrimidine (4.0 g, 16.6 mmol) was obtained the title compound as a tan solid. ¹H NMR (400 MHz, DMSO-d₆) δ 5.63 (s, 2H), 7.19-7.30 (m, 5H), 7.41 (dd, J=2, 9 Hz, 1H), 7.63 (d, J=9 Hz, 1H), 7.84 (s, 1H), 8.07 (s, 1H), 8.36 (s, 1H), 8.54 (s, 1H), 8.61 (s, 1H).

### Example 7

### 1-Benzyl-N-{5-[(E)-2-pyridin-4-ylethenyl]pyrimidin-4-yl}-1H-indazol-5-amine

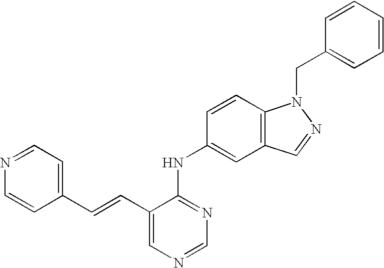

In a similar manner as described in Example 1a, from 4-iodopyridine (62 mg, 0.3 mmol) and 1-benzyl-N-(5-vinylpyrimidin-4-yl)-1H-indazol-5-amine (98 mg, 0.3 mmol) was obtained the title compound as a yellow solid (22 mg, 18%). ¹H NMR (400 MHz, DMSO-d₆) δ 5.64 (s, 2H), 7.18-7.31 (m, 6H), 7.48 (m, 1H), 7.60-7.73 (m, 3H), 7.80 (d, J=6 Hz, 1H), 7.93 (s, 1H), 8.08 (s, 1H), 8.44 (s, 1H), 8.57 (d, J=6 Hz, 1H), 8.64 (s, 1H), 8.70 (d, J=6 Hz, 1H), 9.14 (s, 1H); ESIMS: 405 (M+H)⁺

### Example 8

### 2-((E)-2-{4-[(1-Benzyl-1H-indazol-5-yl)amino]pyrimidin-5-yl}ethenyl)pyridin-3-ol

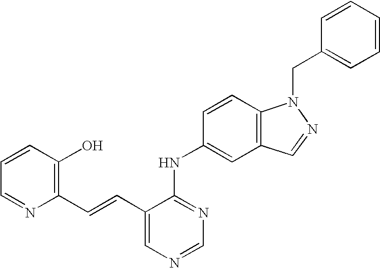

In a similar manner as described in Example 1a, from 2-iodopyridin-3-ol (66 mg, 0.3 mmol) and 1-benzyl-N-(5-vinylpyrimidin-4-yl)-1H-indazol-5-amine (98 mg, 0.3 mmol) was obtained the title compound as a brown solid (15 mg, 12%). ¹H NMR (400 MHz, DMSO-d₆) δ 5.62 (s, 2H), 7.13-7.31 (m, 6H), 7.45-7.62 (m, 4H), 7.82 (d, J=16 Hz, 1H), 7.94 (s, 1H), 8.05-8.10 (m, 2H), 8.42 (s, 1H), 8.53 (s, 1H), 9.18 (s, 1H), 10.16 (s, 1H); ESIMS: 421 (M+H)⁺

### Example 9

### 1-Benzyl-N-{5-[(E)-2-(1H-pyrazol-4-yl)ethenyl]pyrimidin-4-yl}-1H-indazol-5-amine

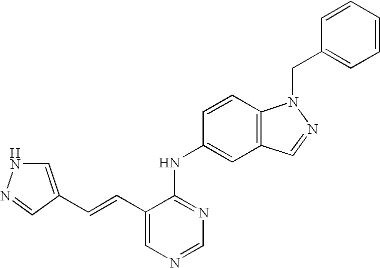

In a similar manner as described in Example 1a, from 1-acetyl-4-iodo-1H-pyrazole (71 mg, 0.3 mmol) and 1-benzyl-N-(5-vinylpyrimidin-4-yl)-1H-indazol-5-amine (98 mg, 0.3 mmol) was obtained the title compound as a tan solid (8 mg, 7%). ¹H NMR (400 MHz, DMSO-d₆) δ 5.64 (s, 2H), 7.09 (d, J=4 Hz, 1H), 7.20-7.32 (m, 9H), 7.50 (dd, J=2, 9 Hz, 1H), 7.64 (d, J=9 Hz, 1H), 7.96 (s, 1H), 8.07 (s, 1H), 8.38 (s, 1H), 8.47 (s, 1H), 8.88 (s, 1H); ESIMS: 394 (M+H)⁺

### Example 10

### N-{5-[(E)-2-(2-Aminopyrimidin-5-yl)ethenyl]pyrimidin-4-yl}-1-benzyl-1H-indazol-5-amine

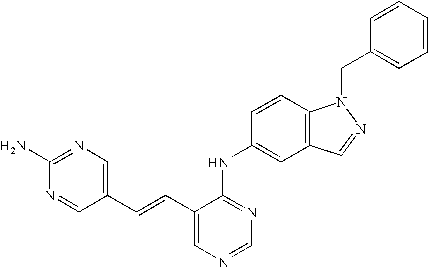

In a similar manner as described in Example 1a, from 5-iodopyrimidin-2-amine (66 mg, 0.3 mmol) and 1-benzyl-N-(5-vinylpyrimidin-4-yl)-1H-indazol-5-amine (98 mg, 0.3 mmol) was obtained the title compound as a tan solid (33 mg, 26%). ¹H NMR (400 MHz, DMSO-d₆) δ 5.64 (s, 2H), 6.89 (s, 2H), 7.03 (d, J=116 Hz, 1H), 7.21-7.32 (m, 6H), 7.50 (dd, J=2, 9 Hz, 1H), 7.65 (d, J=9 Hz, 1H), 7.96 (s, 1H), 8.08 (s, 1H), 8.40 (s, 1H), 8.52 (s, 1H), 8.56 (s, 2H), 8.93 (s, 1H); ESIMS: 419 (M−H)⁻

### Example 11

### N-[3-((E)-2-{4-[(1-Benzyl-1H-indazol-5-yl)amino]pyrimidin-5-yl}ethenyl)phenyl]acetamide

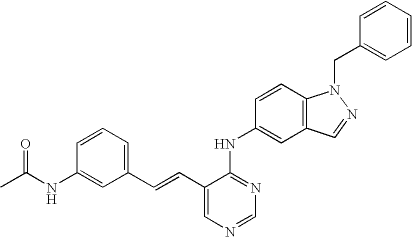

In a similar manner as described in Example 1a, from N-(3-iodophenyl)acetamide (78 mg, 0.3 mmol) and 1-benzyl-N-(5-vinylpyrimidin-4-yl)-1H-indazol-5-amine (98 mg, 0.3 mmol) was obtained the title compound as an amber glass (30 mg, 22%). ¹H NMR (400 MHz, DMSO-d₆) δ 2.03 (s, 3H), 5.63 (s, 2H), 7.16-7.51 (m, 11H), 7.63 (d, J=9 Hz, 1H), 7.76 (s, 1H), 7.94 (s, 1H), 8.06 (s, 1H), 8.41 (s, 1H), 8.59 (s, 1H), 9.09 (s, 1H), 9.97 (s, 1H); ESIMS: 461 (M+H)⁺

### Example 12

### N-(4-Phenoxyphenyl)-5-[(E)-2-phenylethenyl]pyrimidin-4-amine Hydrochloride

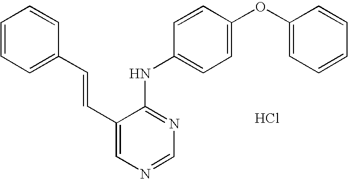

a) In a similar manner as described in Example 1c, mixture of 4-chloro-5-[(E)-2-phenylethenyl]pyrimidine (50 mg, 0.23 mmol) and 4-phenoxyaniline (47 mg, 0.25 mmol) in 2-propanol (5 mL) was heated at 70° C. for 16 h. The mixture was cooled to rt and the resulting precipitate was collected by filtering, washed with cold 2-propanol, with diethyl ether, and dried to give the title compound as a yellow solid (62 mg, 67%). ¹H NMR (400 MHz; DMSO-d₈) δ 7.03-7.19 (m, 5H), 7.37-7.47 (m, 7H), 7.56 (d, J=8 Hz, 2H), 7.73 (d, J=8 Hz, 2H), 8.76 (d, J=4 Hz, 2H), 10.47 (s, 1H), ESIMS: 366 (M+H)⁺

b) 4-Chloro-5-[(E)-2-phenylethenyl]pyrimidine A mixture of (E)-2-phenylethenylboronic acid (0.93 g, 6.2 mmol), 4-chloro-5-iodopyrimidine (1.5 g, 6.2 mmol), 2M sodium carbonate solution (1.5 mL) and 1,1′-bis(diphenylphosphino)ferrocene palladium (II) (0.39 g) in dimethylformamide:toluene 1:2 (15 mL) was heated at 80° C. for 20 h. The reaction mixture was partitioned between dichloromethane and water. The dichloromethane layer was dried, concentrated, and chromatographed on silica gel, eluting with ethyl acetate:hexane 1:9 to give the title compound (0.61 g, 47%). ¹H NMR (400 MHz; DMSO-d₆) δ 7.22 (d, J=116 Hz, 1H), 7.33-7.42 (m, 3H), 7.54 (d, J=16 Hz, 1H), 7.63 (d, J=8 Hz, 2H), 8.89 (s, 1H), 9.22 (s, 1H); ESIMS: 217 (M+H)⁺

### Example 13

### N-{3-Chloro-4-[(3-fluorobenzyl)oxy]phenyl}-5-[(E)-2-pyridin-3-ylethenyl]pyrimidin-4-amine

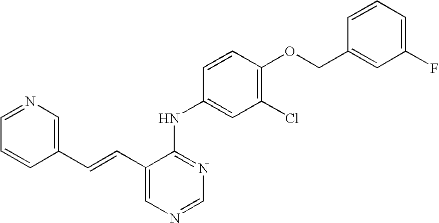

a) In a similar manner as described in Example 1a, from 3-Iodopyridine (62 mg, 0.3 mmol) and N-{3-chloro-4-[(3-fluorobenzyl)oxy]phenyl}-5-vinylpyrimidin-4-amine (107 mg, 0.3 mmol) was obtained the title compound as a brown solid (83 mg, 64%). ¹H NMR (400 MHz; DMSO-d₆) δ 5.25 (s, 2H), 7.17-7.33 (m, 5H), 7.45-7.59 (m, 4H), 7.80 (d, J=2 Hz, 1H), 8.10 (d, J=8 Hz, 1H), 8.53 (m, 2H), 8.67 (s, 1H), 8.86 (s, 1H), 8.99 (s, 1H); ESIMS: 433 (M+H)⁺

b) N-(3-Chloro-4-[(3-fluorobenzyl)oxy]phenyl)-5-ethenyl-4-pyrimidinamine In a similar manner as described in Example 1 b, a mixture of N-(3-Chloro-4-{[(3-fluorophenyl)methyl]oxy}phenyl)-5-iodo-4-pyrimidinamine (2.0 g, 4.7 mmol), vinyltributylstannane (1.8 mL, 6.1 mmol), tetrabutylammonium chloride (1.3 g, 4.7 mmol), palladium acetate (50 mg) and palladium(II) chloride (42 mg) in dimethylformamide (10 mL) was heated at 55° C. for 6 h. The reaction was cooled, diluted with ethyl acetate (200 mL) and water (200 mL) containing potassium fluoride (2.5 g), shaken well and filtered through celite. The ethyl acetate layer was separated, the aqueous extracted with additional ethyl acetate and the combined organic layers were dried, concentrated and chromatographed on silica gel eluting with a ethyl acetate to 9:1 ethyl acetate:methanol gradient to give a straw-colored solid.

c) N-{3-Chloro-4-[(3-fluorobenzyl)oxy]phenyl}-5-iodopyrimidin-4-amine hydrochloride To a stirred solution containing 2.0 g (8.33 mmol) of iodochloropyrimidine and 20 mL of isopropanol was added 2.2 g (8.73 mmol) of aniline. The reaction mixture was heated at 80° C. for 13 h, then cooled to room temperature and filtered. The filter cake was washed with isopropanol, collected, and dried under reduced pressure to give the title compound as a tan solid (3.53 g, 86%). ¹H-NMR (300 MHz, DMSO-d₆) δ 4.56 (brs, 1H), 5.26 (s, 2H), 7.17 (dd, 1H, J=8.1 and 8.1 Hz), 7.24 (d, 1H, J=9.0 Hz), 7.27-7.32 (m, 2H), 7.39-7.50 (m, 2H), 7.64 (d, 1H, J=2.4 Hz), 8.63 (s, 1H), 8.77 (s, 1H), and 9.31 (brs, 1H); ESIMS: 456.0 (M+H⁺).

### Example 14

### N-{3-Chloro-4-[(3-fluorobenzyl)oxy]phenyl}-5-[(E)-2-pyridin-4-ylethenyl]pyrimidin-4-amine

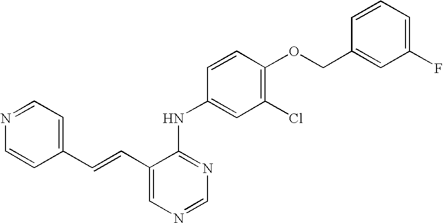

In a similar manner as described in Example 1a, from 4-iodopyridine (62 mg, 0.3 mmol) and N-{3-chloro-4-[(3-fluorobenzyl)oxy]phenyl}-5-vinylpyrimidin-4-amine (107 mg, 0.3 mmol) was obtained the title compound as a brown solid (8 mg, 6%). ¹H NMR (400 MHz; DMSO-d₆) δ 5.23 (s, 2H), 7.14-7.31 (m, 5H), 7.42-7.54 (m, 2H), 7.60 (d, J=6 Hz, 2H), 7.66 (d, J=16 Hz, 1H), 7.76 (d, J=3 Hz, 1H), 8.51 (s, 1H), 8.58 (d, J=6 Hz, 2H), 8.66 (s, 1H), 9.04 (s, 1H); ESIMS: 433 (M+H)⁺

### Example 15

### 2-{(E)-2-[4-({3-Chloro-4-[(3-fluorobenzyl)oxy]phenyl}amino)pyrimidin-5-yl]ethenyl}pyridin-3-ol

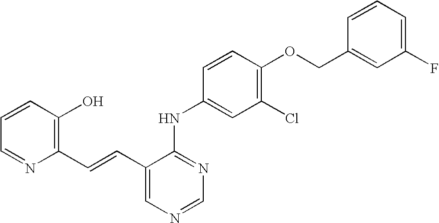

In a similar manner as described in Example 1a, from 2-iodopyridin-3-ol (66 mg, 0.3 mmol) and N-{3-chloro-4-[(3-fluorobenzyl)oxy]phenyl}-5-vinylpyrimidin-4-amine (107 mg, 0.3 mmol) was obtained the title compound as a brown solid (30 mg, 22%). ¹H NMR (400 MHz; DMSO-d₆) δ 5.24 (s, 2H), 7.16-7.34 (m, 5H), 7.44-7.60 (m, 3H), 7.79-7.96 (m, 2H), 7.96 (s, 1H), 8.14 (d, J=4 Hz, 1H), 8.52 (s, 1H), 8.60 (s, 1H), 9.14 (s, 1H), 10.21 (s, 1H); ESIMS: 449 (M+H)⁺

### Example 16

### N-{3-Chloro-4-[(3-fluorobenzyl)oxy]phenyl}-5-[(E)-2-thien-2-ylethenyl]pyrimidin-4-amine

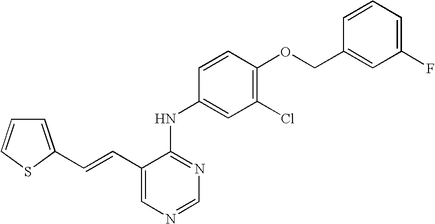

In a similar manner as described in Example 1a, from 2-iodothiophene (63 mg, 0.3 mmol) and N-{3-chloro-4-[(3-fluorobenzyl)oxy]phenyl}-5-vinylpyrimidin-4-amine (107 mg, 0.3 mmol) was obtained the title compound as an orange solid (21 mg, 16%). ¹H NMR (400 MHz; DMSO-d₆) δ 5.25 (s, 2H), 7.02-7.34 (m, 7H), 7.43-7.56 (m, 4H), 7.79 (s, 1H), 8.49 (s, 1H), 8.60 (s, 1H), 9.01 (s, 1H); ESIMS: 438 (M+H)⁺

### Example 17

### N-{3-Chloro-4-[(3-fluorobenzyl)oxy]phenyl}-5-[(E)-2-thien-3-yletheny]pyrimidin-4-amine

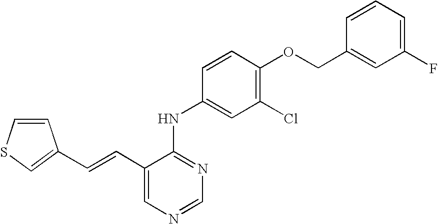

In a similar manner as described in Example 1a, from 3-iodothiophene (63 mg, 0.3 mmol) and N-{3-chloro-4-[(3-fluorobenzyl)oxy]phenyl}-5-vinylpyrimidin-4-amine (107 mg, 0.3 mmol) was obtained the title compound as a tan foam (79 mg, 60%). ¹H NMR (400 MHz; DMSO-d₆) δ 5.25 (s, 2H), 7.19-7.35 (m, 7H), 7.41-7.64 (m, 4H), 7.80 (d, J=2 Hz, 1H), 8.50 (s, 1H), 8.58 (s, 1H), 8.91 (s, 1H); ESIMS: 438 (M+H)⁺

### Example 18

### 5-{(E)-2-[4-({3-Chloro-4-[(3-fluorobenzyl)oxy]phenyl}amino)pyrimidin-5-yl]ethenyl}pyrimidin-2-amine

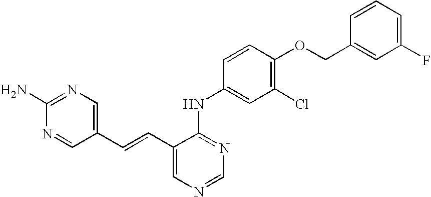

In a similar manner as described in Example 1a, from 5-iodopyrimidine-2-amine (66 mg, 0.3 mmol) and N-{3-chloro-4-[(3-fluorobenzyl)oxy]phenyl}-5-vinylpyrimidin-4-amine (107 mg, 0.3 mmol) was obtained the title compound as a tan solid (49 mg, 36%). ¹H NMR (400 MHz; DMSO-d₆) δ 5.22 (s, 2H), 6.90 (s, 1H), 7.02 (d, J=16 Hz, 1H), 7.16-7.31 (m, 6H), 7.42-7.48 (m, 1H), 7.52-7.56 (m, 1H), 7.77 (d, J=2 Hz, 1H), 8.46 (s, 1H), 8.54 (d, J=4 Hz, 2H), 8.55 (s, 1H), 8.83 (s, 1H); ESIMS: 449 (M+H)⁺

### Example 19

### N-{3-Chloro-4-[(3-fluorobenzyl)oxy]phenyl}-5-[(E)-2-(1H-pyrazol-4-yl)ethenyl]pyrimidin-4-amine

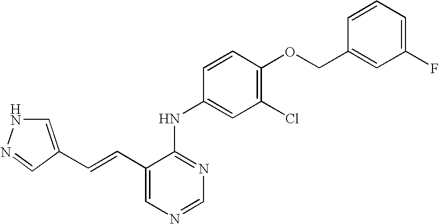

In a similar manner as described in Example 1a, from 1-acetyl-4-iodo-1H-indazol-5-amine (71 mg, 0.3 mmol) and N-{3-chloro-4-[(3-fluorobenzyl)oxy]phenyl}-5-vinylpyrimidin-4-amine (107 mg, 0.3 mmol) was obtained the title compound as a yellow solid (8 mg, 6%). ¹H NMR (400 MHz; DMSO-d₆) δ 5.22 (s, 2H), 7.06 (d, J=6 Hz, 2H), 7.11-7.31 (m, 6H), 7.42-7.55 (m, 2H), 7.77 (d, J=3 Hz, 1H), 7.87 (br s, 1H), 8.46 (s, 1H), 8.50 (s, 1H), 8.88 (s, 1H); ESIMS: 422 (M+H)⁺

### Example 20

### N-(3-{(E)-2-[4-({3-Chloro-4-[(3-fluorobenzyl)oxy]phenyl}amino)pyrimidin-5-yl]ethenyl}phenyl)acetamide

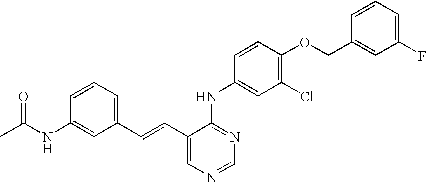

In a similar manner as described in Example 1a, from N-(3-iodophenyl)acetamide (78 mg, 0.3 mmol) and N-{3-chloro-4-[(3-fluorobenzyl)oxy]phenyl}-5-vinylpyrimidin-4-amine (107 mg, 0.3 mmol) was obtained the title compound as a yellow solid (55 mg, 37%). ¹H NMR (400 MHz; DMSO-d₈) δ 2.04 (s, 3H), 5.22 (s, 2H), 7.16-7.55 (m, 11H), 7.77 (m, 2H), 8.48 (s, 1H), 8.62 (s, 1H), 9.01 (s, 1H), 9.99 (s, 1H); ESIMS: 489 (M+H)⁺

### Example 21

### N-{3-Chloro-4-[(3-fluorobenzyl)oxy]phenyl}-5-[(E)-2-(3,4-dimethoxyphenyl) ethenyl]pyrimidin-4-amine

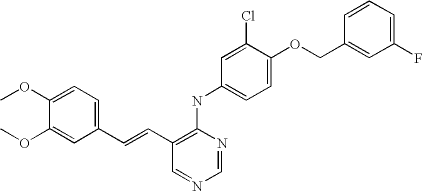

A mixture of N-{3-chloro-4-[(3-fluorobenzyl)oxy]phenyl}-5-iodopyrimidin-4-amine (228 mg, 0.5 mmol), dimethoxy styrene (0.11 mL, 0.75 mmol), Pd(OAc)₂(PPh₃)₂ (18.7 mg, 0.025 mmol), NaOAc (123 mg, 1.5 mmol), and Bu₄NCl (152.8 mg, 0.55 mmol) in 1.5 mL degassed DMF was heated at 95° C. for three days. Water and ethyl acetate was added and the aqueous layer extracted with ethyl acetate. The organic layer was washed with brine, dried with Na₂SO₄, filtered, and evaporated in vacuo. Purification via preparative TLC (50% Ethyl Acetate/hexanes) provided the title compound as a yellow film (23 mg, 9%). ¹H NMR (300 MHz, DMSO-d₆) δ 3.78 (s, 3H), 3.82 (s, 3H), 5.23 (s, 2H), 7.0-6.98 (m, 1H), 7.23-7.18 (m, 5H), 7.32-7.28 (m, 3H), 7.81-7.79 (m, 1H), 8.47 (s, 1H), 8.57 (s, 1H), 8.89-8.91 (m, 1H); ESIMS: 492 (M+H)⁺

### Example 22

### N-{3-Chloro-4-[(3-fluorobenzyl)oxy]phenyl}-5-[(E)-2-phenylethenyl]pyrimidin-4-amine

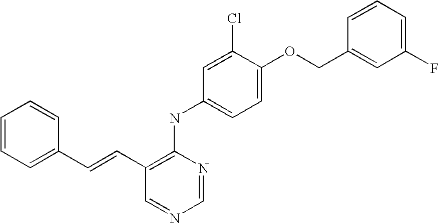

In a similar manner as described in Example 1a, from iodobenzene (0.123 mL, 1.10 mmol) and N-{3-chloro-4-[(3-fluorobenzyl)oxy]phenyl}-5-vinylpyrimidin-4-amine (107 mg, 0.3 mmol) was obtained the title compound as a yellow solid (21.8 mg, 22%). ESIMS: 432 (M+H)⁺

### Example 23

### N-(5-{(E)-2-[4-({3-Chloro-4-[(3-fluorobenzyl)oxy]phenyl}amino)pyrimidin-5-yl]ethenyl}pyridin-2-yl)acetamide

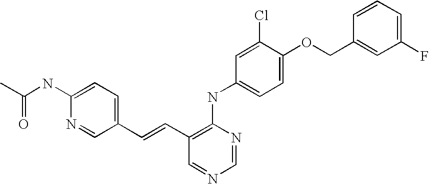

In a similar manner as described in Example 1a, 5-bromo-2-acetamidopyridine (236 mg, 1.10 mmol) and N-{3-chloro-4-[(3-fluorobenzyl)oxy]phenyl}-5-vinylpyrimidin-4-amine (107 mg, 0.3 mmol) was obtained the title compound as a yellow solid (17.9 mg, 33%). ¹H NMR (300 MHz, DMSO-d₆) δ 2.10 (s, 3H), 5.23 (s, 2H), 7.54-7.17 (m, 7H), 7.78 (d, J=2.56 Hz, 1H), 8.14-8.07 (m, 3H), 8.66-8.49 (m, 3H), 8.93 (s, 1H); ESIMS: 490 (M+H)⁺

### Example 24

### N-{3-Chloro-4-[(3-fluorobenzyl)oxy]phenyl}-5-(thien-2-ylethynyl)pyrimidin-4-amine

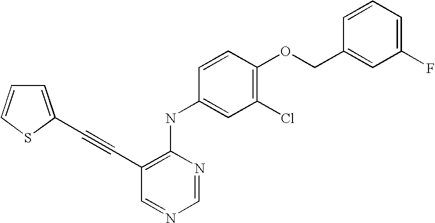

a) A mixture of N-{3-chloro-4-[(3-fluorobenzyl)oxy]phenyl}-5-ethynylpyrimidin-4-amine (41 mg, 0.12 mmol), 2-iodothiophene (0.03 ml, 0.23 mmol), Pd(PPh₃)₂Cl₂ (8.1 mg, 0.01 mmol), PPh₃ (1.5 mg, 0.006 mmol), CuI (0.5 mg, 0.003 mmol), and TEA (0.04 mL, 0.3 mmol) in 3 mL THF was heated at reflux for 6 h. Water was added and the mixture extracted with ethyl acetate. The organic layer was washed with brine, dried with Na₂SO₄, filtered, and evacuated in vacuo. Purification via column chromatography (30% ethyl acetate/hexanes) provided the title compound as a yellow solid (38 mg, 75%). ¹H NMR (300 MHz, DMSO-d₆) δ 5.19 (s, 2H), 7.18 9m, 3H), 7.25 (m, 2H), 7.41 (m, 2H), 7.68 (m, 2H), 8.45 (s, 1H), 8.48 (s, 1H), 9.04 (s, 1H); ESIMS: (M+H)⁺

b) N-{3-Chloro-4-[(3-fluorobenzyl)oxy]phenyl}-5-ethynylpyrimidin-4-amine To a stirred solution containing 3.88 g (9.11 mmol) of N-{3-chloro-4-[(3-fluorobenzyl)oxy]phenyl}-5-[(trimethylsilyl)ethynyl]pyrimidin-4-amine and 150 mL of THF at 0° C. was added 18.2 mL (18.2 mmol) of 1M TBAF in THF. The reaction mixture was allowed to stir at this temperature for 30 min and quenched by the addition of water. The mixture was extracted with diethyl ether and the combined organic layers were washed with brine and dried over MgSO₄. The solvents were removed under reduced pressure and the residue was subjected to silca gel chromatography to give the title compound as a tan solid (3.0 g, 94%). ¹H-NMR (300 MHz, CDCl₃) δ 3.71 (s, 1H), 5.19 (s, 2H), 6.98 (d, 1H, J=9.0 Hz), 7.06 (dd, 1H, J=9.0 Hz), 7.22-7.30 (m, 3H), 7.39 (ddd, 1H, J=14.1, 8.1, and 6.0 Hz), 7.47 (dd, 1H, J=8.7 and 2.7 Hz), 7.77 (d, 1H, J=3.0 Hz), 8.49 (s, 1H), and 8.69 (s, 1H).

c) N-{3-Chloro-4-[(3-fluorobenzyl)oxy]phenyl}-5-[(trimethylsilyl)ethynyl]pyrimidin-4-amine To a stirred solution containing 5.0 g (11.0 mmol) of N-{3-chloro-4-[(3-fluorobenzyl)oxy]phenyl}-5-iodopyrimidin-4-amine in 100 mL of THF was added 1.85 mL (13.2 mmol) of trimethylsilylacetylene, 155 mg (0.22 mmol) of dichloropalladium-bistriphenylphosphine, 85 mg (0.44 mmol) of copper (I) iodide, and 6.5 mL (46.2 mmol) of triethyl amine. The reaction mixture was heated at 60° C. for 3 h, then allowed to cool to room temperature. The mixture was diluted with ethyl acetate and washed with water and brine, and dried over NaSO₄. The solvents were removed under reduced pressure and the residue subjected to silica gel chromatography to give the title compound as a brown oil (3.89 g, 83%). ¹H-NMR (300 MHz, CDCl₃) δ 0.31 (s, 9H), 5.15 (s, 2H), 6.95 (d, 1H, J=9.0 Hz), 7.01 (dd, 1H, J=8.7 and 8.7 Hz), 7.18-7.27 (m, 3H), 7.32-7.42 (m, 2H), 7.74 (d, 1H, J=2.7 Hz), 8.37 (brs), and 8.70 (brs).

### Example 25

### N-{3-Chloro-4-[(3-fluorobenzyl)oxy]phenyl}-5-(pyridin-3-ylethynyl)pyrimidin-4-amine

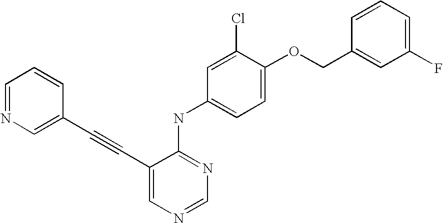

In a similar manner as described in Example 24a, from 3-iodopyridine (32.6 mg, 0.16 mmol) was obtained the title compound as a brown solid (20 mg, 41%). ¹H NMR (300 MHz, DMSO-d₆) δ 5.19 (s, 2H), 7.27-7.10 (m, 5H), 7.49-7.38 (m, 5H), 7.71 (m, 1H), 8.55 (bs, 1H), 9.31 (s, 1H); ESIMS: 431 (M+H)⁺

### Example 26

### N-{3-Chloro-4-[(3-fluorobenzyl)oxy]phenyl}-5-[(1-methyl-1H-imidazol-5-yl)ethynyl]pyrimidin-4-amine

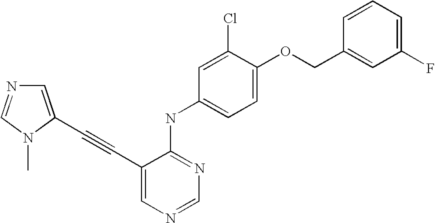

In a similar manner as described in Example 24a, from 5-iodo-1-methyl-imidazole (83.2 mg, 0.4 mmol) was obtained the title compound as a brown solid (52 mg, 60%). ¹H NMR (300 MHz, DMSO-d₆) δ 3.77 (s, 3H), 5.27 (s, 2H), 5.78 (s, 2H), 7.26 (m, 4H), 7.50 (m, 4H), 8.58 (m, 2H), 9.05 (s, 1H); ESIMS: 434 (M+H)⁺

### Example 27

### N-{3-Chloro-4-[(3-fluorobenzyl)oxy]phenyl}-5-(1H-pyrazol-4-ylethynyl)pyrimidin-4-amine

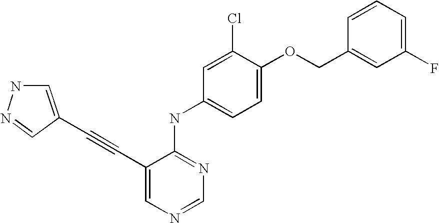

In a similar manner as described in Example 24a, from 4-iodopyrazole (77.6 mg, 0.4 mmol) was obtained the title compound as a brown solid (21 mg, 25%). ¹H NMR (300 MHz, DMSO-d₆) δ 5.19 (s, 2H), 7.16 (m, 2H), 7.25 9m, 2H), 7.41 (m, 1H), 7.49 (m, 1H), 7.71 (m, 1H), 7.76 (m, 1H), 8.13 (m, 1H), 8.38 (s, 1H), 8.46 (s, 1H), 8.83 (s, 1H); ESIMS: 420 (M+H)⁺

### Example 28

### N-{-3-Chloro-4-[(3-fluorobenzyl)oxy]phenyl}-5-(pyrimidin-5-ylethynyl)pyrimidin-4-amine

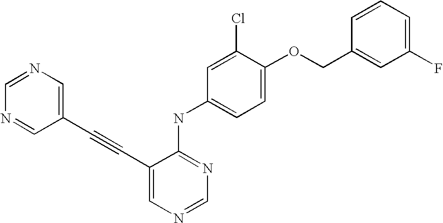

In a similar manner as described in Example 24a, from 5-bromopyrimidine (63.6 mg, 0.4 mmol) was obtained the title compound as a tan solid (31 mg, 36%). ¹H NMR (300 MHz, DMSO-d₆) δ 5.20 (s, 2H), 7.27-7.21 (m, 3H), 7.59-7.47 (m, 5H), 7.69 (d, J=2.8 Hz, 1H), 8.52 (d, J=5.5 Hz, 2H), 9.05 (s, 1H), 9.17 (s, 1H); ESIMS: 432 (M+H)⁺

### Example 29

### N-{3-Chloro-4-[(3-fluorobenzyl)oxy]phenyl}-5-(1,3-thiazol-2-ylethynyl)pyrimidin-4-amine

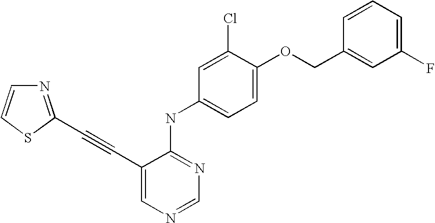

In a similar manner as described in Example 24a, from 2-bromothiazole (0.018 mL, 0.2 mmol) was obtained the title compound as a brown solid (19 mg, 44%). ¹H NMR (300 MHz, DMSO-d₆) δ 5.19 (s, 2H), 7.19 (m, 4H), 7.43 (m, 2H), 7.67 (m, 1H), 7.94 (m, 2H), 8.54 (m, 2H), 9.28 (s, 1H); ESIMS: 437 (M+H)⁺

### Example 30

### N-{3-Chloro-4-[(3-fluorobenzyl)oxy]phenyl}-5-(thien-3-ylethynyl)pyrimidin-4-amine

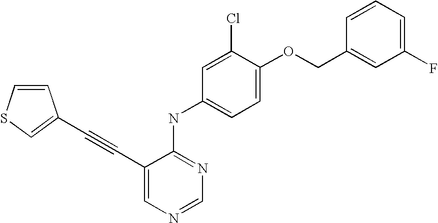

In a similar manner as described in Example 24a, from 3-iodothiophene (42 mg, 0.2 mmol) was obtained the title compound as a tan solid (14 mg, 34%). ¹H NMR (300 MHz, DMSO-d₆) δ 5.20 (s, 2H), 7.17 (m, 2H), 7.26 (m, 2H), 7.31 (m, 1H), 7.41 (m, 1H), 7.47 (m, 1H), 7.62 (m, 1H), 7.71 (m, 1H), 7.95 (m, 1H), 8.43 (s, 1H), 8.48 (s, 1H), 8.92 (s, 1H); ESIMS: 436 (M+H)⁺

### Example 31

### N-{3-Chloro-4-[(3-fluorobenzyl)oxy]phenyl}-5-[(2-morpholin-4-ylpyrimidin-4-yl)ethynyl]pyrimidin-4-amine

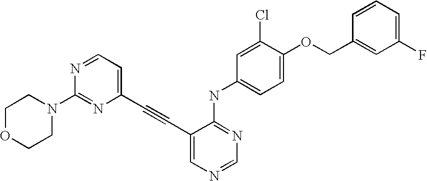

In a similar manner as described in Example 24a, from 4-(4-iodo-2-pyrimidinyl)morpholine (58 mg, 0.2 mmol) was obtained the title compound as a tan solid (16 mg, 31%). ¹H NMR (300 MHz, DMSO-d₆) δ 3.66-3.69 (m, 8H), 5.24 (s, 2H), 7.21-7.23 (m, 1H), 7.30-7.31 (m, 1H), 7.63-7.54 (m, 6H), 8.46-8.47 (m, 1H), 8.58-8.57 (m, 2H), 9.16 (m, 1H); ESIMS: 517 (M+H)⁺

### Example 32

### N-(3-Chloro-4-{[3-fluorophenyl)methyl]oxy}phenyl)-5-(2-pyrimidinylethynyl)-4-pyrimidinamine

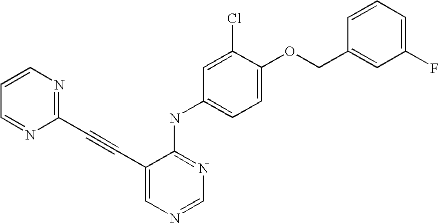

In a similar manner as described In Example 24a, from 2-bromopyridine (24 mg, 0.15 mmol) was obtained the title compound as a brown solid (14 mg, 32%). ESIMS: 431 (M+H)⁺

### Example 33

### 5-[(6-Amino-3-pyridinyl)ethynyl]-N-(3-chloro-4-{[(3-fluorophenyl)methyl]oxy}phenyl)-4-pyrimidinamine

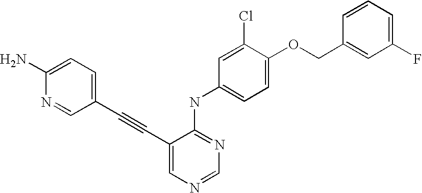

In a similar manner as described in Example 24a, from 2-amino-5-iodopyridine (105 mg, 0.48 mmol) was obtained the title compound as a tan solid (18 mg, 35%). ESIMS: 446 (M+H)⁺

### Example 34

### N-{3-Chloro-4-[(3-fluorobenzyl)oxy]phenyl}-5-[(3-fluorophenyl) ethynyl]pyrimidin-4-amine

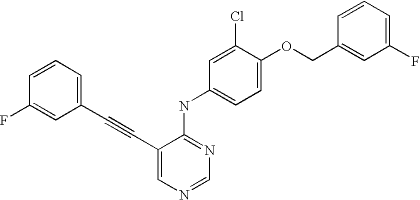

In a similar manner as described in Example 24a, from 3-fluoro-iodobenzene (0.018 mL, 0.16 mmol) was obtained the title compound as a brwon solid (20 mg, 30%). ¹H NMR (300 MHz, DMSO-d₆) δ 5.25 (s, 2H), 7.67-7.16 (m, 10H), 7.74-7.73 (m, 1H), 8.56 (bs, 2H), 9.01 (s, 1H); ESIMS: 448 (M+H)⁺

### Example 35

### 4-{[4-({3-Chloro-4-[(3-fluorobenzyl)oxy]phenyl}amino)pyrimidin-5-yl]ethynyl}phenol

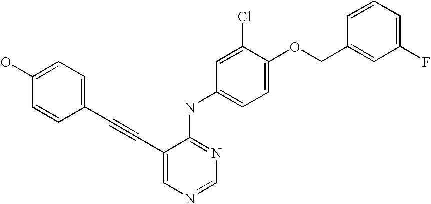

In a similar manner as described in Example 24a, from 4-iodophenol (80.4 mg, 0.365 mmol) was obtained the title compound as a tan solid (40 mg, 34%). ¹H NMR (300 MHz, DMSO-d₆) δ 2.83 (s, 3H), 5.26 (s, 2H), 6.91-6.88 (m, 2H), 7.08-7.13 (m, 1H), 7.19-7.17 (d, J=8.79 Hz, 1H), 7.36-7.30 (m, 2H), 7.50-7.42 (m, 3H), 7.62-7.60 (m, 1H), 7.92 (m, 1H), 8.36 (s, 1H), 8.45 (s, 1H), 8.53 (s, 1H), 8.92 (bs, 1H); ESIMS: 446 (M+H)⁺

### Example 36

### N-{3-Chloro-4-[(3-fluorobenzyl)oxy]phenyl}-5-[(6-methoxypyridin-2-yl)ethynyl]pyrimidin-4-amine

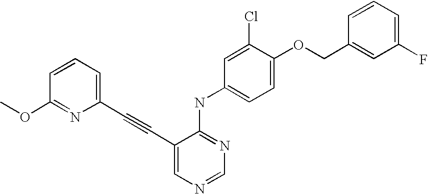

In a similar manner as described in Example 24a, from 2-bromo-6-methoxypyridine (0.025 mL, 0.2 mmol) was obtained the title compound as a brown solid (11 mg, 20%). ¹H NMR (300 MHz, DMSO-d₆) δ 4.37 (s, 3H), 5.71 (s, 2H), 7.29 (d, J=8.4 Hz, 1H), 7.53-7.57 (m, 1H), 7.64 (d, J=8.97 Hz, 1H), 7.81-7.76 (m, 3H), 7.92-7.87 (m, 1H), 8.02-8.06 (m, 1H), 8.20-8.17 (dd, J=0.91, 7.33 Hz, 1H), 8.36-8.34 (m, 1H), 9.03-8.93 (m, 3H); ESIMS: 461 (M+H)⁺

### Example 37

### 5-[(3-Aminophenyl)ethynyl]-N-{3-chloro-4-[(3-fluorobenzyl)oxy]phenyl}pyrimidin-4-amine

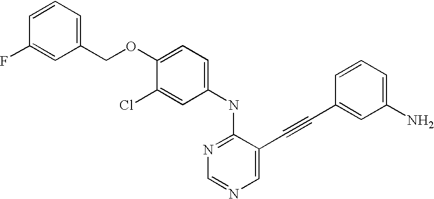

In a similar manner as described in Example 24a, from 3-iodoaniline (0.2 mL, 1.7 mmol) was obtained the title compound as a tan solid (480 mg, 76%). ¹H-NMR (300 MHz, CDCl₃) δ 3.82 (brs, 2H), 5.16 (s, 2H), 6.76 (dd, 1H, J=8.1 and 1.5 Hz), 6.87-6.89 (m, 2H), 6.97-7.00 (m, 2H), 7.05 (ddd, 1H, J=8.7, 8.7, and 1.8 Hz), 7.19-7.31 (m, 3H), 7.39 (ddd, 1H, J=13.8, 7.8, and 6.0 Hz), 7.47 (dd, 1H, J=8.7 and 2.7 Hz), 7.79 (d, 1H, J=2.7 Hz), 8.60 (brs, 1H), and 8.70 (brs, 1H).

### Example 38

### N-(3-{[4-({3-Chloro-4-[(3-fluorobenzyl)oxy]phenyl}amino)pyrimidin-5-yl]ethynyl}phenyl)acetamide

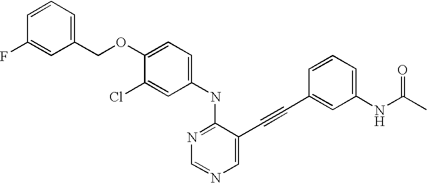

To a stirred solution containing 88 mg (0.198 mmol) of 5-[(3-aminophenyl)ethynyl]-N-{3-chloro-4-[(3-fluorobenzyl)oxy]phenyl}pyrimidin-4-amine (Example 37) in 5 mL of DMF was added 28 μL (0.297 mmol) of acetic anhydride. The reaction mixture was heated at 80° C. for 13 h and allowed to cool to room temperature. The mixture was diluted with diethyl ether, washed with water and brine, and dried over MgSO₄. The solvent was removed under reduced pressure and the residue was subjected to silica gel chromatography to give the title compound as an oil (76 mg, 79%). ¹H-NMR (300 MHz, DMSO-d₆) δ 2.05 (s, 3H), 5.24 (s, 2H), 7.17 (dd, 1H, J=8.4 and 8.4 Hz), 7.22 (d, 1H, J=8.7 Hz), 7.27-7.37 (m, 3H), 7.42-7.56 (m, 4H), 7.75 (d, 1H, J=2.1 Hz), 7.93 (s, 1H), 8.54 (d, 1H, J=3.0 Hz), 9.06 (s, 1H), and 10.07 (s, 1H).

### Example 39

### N-(3-{[4-({3-Chloro-4-[(3-fluorobenzyl)oxy]phenyl}amino)pyrimidin-5-yl]ethynyl}phenyl)ethanethioamide

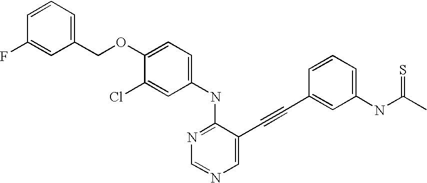

To a stirred solution containing 70 mg (0.144 mmol) of N-(3-{[4-({3-chloro-4-[(3-fluorobenzyl)oxy]phenyl}amino)pyrimidin-5-yl]ethynyl}phenyl)acetamide (Example 38) in 5 mL of toluene was added 32 mg (0.079 mmol) of Lawesson's reagent. The reaction mixture was heated to 100° C. for 6 h, then allowed to cool to room temperature. The solvent was removed under reduced pressure and the residue was subjected to silica gel chromatography to give the title compound as a tan solid (33 mg, 46%): ¹H NMR (300 MHz, DMSO-d₆) δ 2.61 (s, 3H), 5.24 (s, 2H), 7.13-7.32 (m, 5H), 7.42-7.56 (m, 5H), 8.09 (s, 1H), 8.53 (d, 1H, J=3.0 Hz), 9.04 (s, 1H), and 11.66 (brs, 1H); ESIMS: 503.2.

### Example 40

### 2-{[4-({3-Chloro-4-[(3-fluorobenzyl)oxy]phenyl}amino)pyrimidin-5-yl]ethynyl}benzonitrile

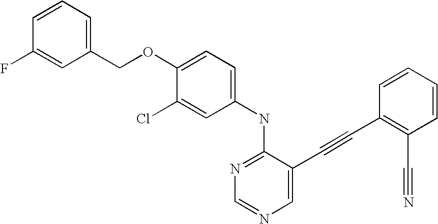

In a similar manner as described in Example 24a, from 2-iodobenzonitrile (20 mg, 0.028 mmol) was obtained the title compound as a brown solid (302 mg, 47%). ¹H NMR (300 MHz, DMSO-d₆) δ 5.27 (s, 2H), 7.17-7.35 (m, 4H), 7.49 (dd, 1H, J=8.4 and 8.4 Hz), 7.58 (dd, 1H, J=8.7 and 2.7 Hz), 7.68 (ddd, 1H, J=9.0, 9.0, and 0.6 Hz), 7.81-7.87 (m, 2H), 7.95 (d, 1H, J=7.5 Hz), 8.00 (d, 1H, J=7.5 Hz), 8.56 (s, 1H), 8.64 (s, 1H), and 8.97 (brs, 1H); ESIMS: 455.1 (M+H)⁺

### Example 41

### 3-{[4-({3-Chloro-4-[(3-fluorobenzyl)oxy]phenyl}amino)pyrimidin-5-yl]ethynyl}benzonitrile

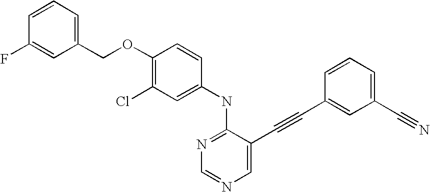

In a similar manner as described in Example 24a, from 3-iodobenzonitrile (20 mg, 0.028 mmol) was obtained the title compound as a brown solid (125 mg, 20%). ¹H NMR (300 MHz, DMSO-d₆) δ 5.25 (s, 2H), 7.14-7.33 (m, 4H), 7.42-7.52 (m, 2H), 7.67 (dd, 1H, J=9.5 and 9.5 Hz), 7.73 (d, 1H, J=2.4 Hz), 7.91 (d, 1H, J=8.1 Hz), 7.98 (d, 1H, J=8.1 Hz), 8.21 (s, 1H), 8.54 (d, 1H, J=4.2 Hz), and 9.02 (s, 1H); ESIMS: 455.1 (M+H)⁺

### Example 42

### 3-{[4-({3-Chloro-4-[(3-fluorobenzyl)oxy]phenyl}amino)pyrimidin-5-yl]ethynyl}benzaldehyde

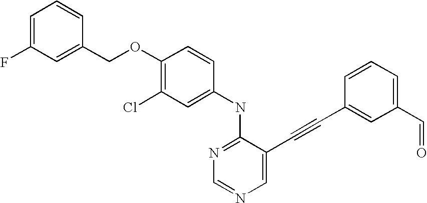

In a similar manner as described in Example 24a, from 3-iodobenzaldehyde (12 mg, 0.017 mmol) was obtained the title compound as an oil (270 mg, 70%). ¹H NMR (300 MHz, DMSO-d₆) δ 5.25 (s, 2H), 7.17 (dd, 1H, J=8.1 Hz), 7.23 (d, 1H, J=9.0 Hz), 7.27-7.33 (m, 2H), 7.42-7.53 (m, 2H), 7.69 (dd, 1H, J=7.5 Hz), 7.73 (d, 1H, J=2.4 Hz), 7.98 (m, 2H), 8.22 (s, 1H), 8.56 (brs, 1H), 9.09 (s, 1H), and 10.04 (s, 1H); ESIMS: 457.1.

### Example 43

### N-{3-Chloro-4-[(3-fluorobenzyl)oxy]phenyl}-5-(phenylethynyl)pyrimidin-4-amine

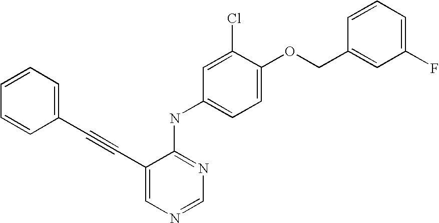

In a similar manner as described in Example 24c, from phenyl-acetylene (0.3 ml, 0.26 mmol) was obtained the title compound as a tan solid (22 mg, 20%). ¹H NMR (300 MHz, DMSO-d₆) δ 5.23 (s, 2H), 7.14-7.32 (m, 3H), 7.42-7.53 (m, 6H), 7.66-7.70 (m, 2H), 7.75 (m, 1H), 8.54 (bs, 2H), 9.00 (s, 1H); ESI MS: 430 (M+H)⁺

### Example 44

### N-{3-Chloro-4-[(3-fluorobenzyl)oxy]phenyl}-5-(pyridin-2-ylethynyl)pyrimidin-4-amine

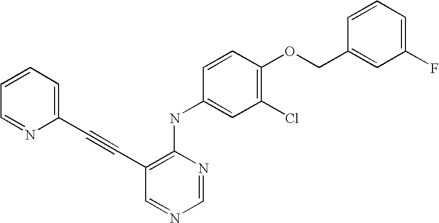

In a similar manner as described in Example 24c, from 2-ethynyl pyridine (46.2 mg, 0.45 mmol) was obtained the title compound as a tan solid (100 mg, 78%). ¹H NMR (300 MHz, DMSO-d₆) δ 5.20 (s, 2H), 7.10-7.19 (m, 2H), 7.24-7.27 (m, 2H), 7.38-7.42 (m, 2H), 7.48-7.49 (m, 1H), 7.70 (m, 1H), 7.76-7.78 (m, 1H), 7.86-7.82 (m, 1H), 8.52 (s, 2H), 8.59 (m, 1H), 9.07 (s, 1H); ESIMS: 431 (M+H)⁺

### Example 45

### 5-[(4-Aminophenyl)ethynyl]-N-{3-chloro-4-[(3-fluorobenzyl)oxy]phenyl}pyrimidin-4-amine

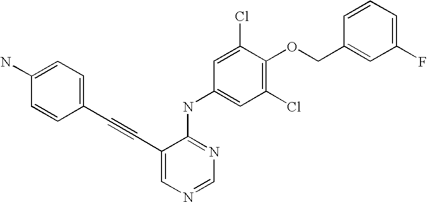

In a similar manner as described in Example 24c, from 4-ethynyl aniline (33.2 mg, 0.283 mmol) was obtained the title compound as a tan solid (63 mg, 68%). ¹H NMR (300 MHz, DMSO-d₆) δ 5.24 (s, 2H), 5.63 (s, 1H), 6.57-6.55 (m, 2H), 7.22-7.20 (m, 2H), 7.34-7.28 (m, 5H), 7.54-7.43 (m, 2H), 7.77 (m, 1H), 8.41 (s, 1H), 8.48 (s, 1H), 8.80 (m, 1H); ESIMS: 445 (M+H)⁺

### Example 46

### N-(3-{[4-({3-Chloro-4-[(3-fluorobenzyl)oxy]phenyl}amino)pyrimidin-5-yl]ethynyl}phenyl)-3-(methylthio)propanamide

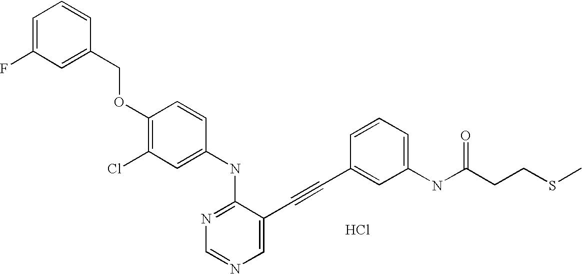

To a stirred solution containing 5-[(3-aminophenyl)ethynyl]-N-3-chloro-4-[(3-fluorobenzyl)oxy]phenyl)pyrimidin-4-amine (Example 37) (136 mg, 0.306 mmol) and 5 mL of dichloromethane at 0° C. was added 51 mg (0.367 mmol) of 3-(methylthio)propanoyl chloride. The ice bath was removed and the reaction mixture was allowed to stir for 15 min, then filtered. The solvents were removed under reduced pressure to give the title compound as a tan solid (152 mg, 85%). ¹H NMR (300 MHz, DMSO-d₆) δ 2.08 (s, 3H), 2.62 (t, 2H, J=6.6 Hz), 2.74 (t, 2H, J=6.6 Hz), 5.26 (s, 2H), 7.17 (dd, 1H, J=9.3 Hz and 9.3 Hz), 7.25-7.32 (m, 3H), 7.38-7.50 (m, 4H), 7.55 (d, 1H, J=3.9 Hz), 7.70 (s, 1H), 8.03 (s, 1H), 8.69 (s, 1H), 9.85 (brs, 1H), and 10.19 (s, 1H); ESIMS: 585.2 (M+H)⁺

### Example 47

### N-{3-Chloro-4-[(3-fluorobenzyl)oxy]phenyl}-5-({1-[(4-methylphenyl)sulfonyl]-1H-indol-6-yl}ethynyl)pyrimidin-4-amine

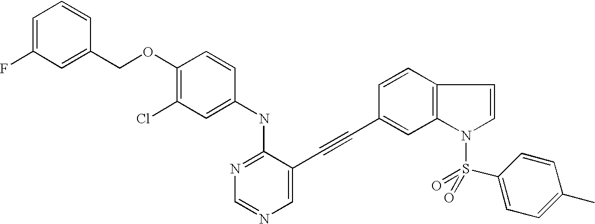

a) To a solution containing 244 mg (0.496 mmol) of N-{3-chloro-4-[(3-fluorobenzyl)oxy]phenyl}-5-iodopyrimidin-4-amine (Example 13c), 176 mg (0.595 mmol) of 6-ethynyl-1-[(4-methylphenyl)sulfonyl]-1H-indole, and 10 mL of THF was added 7 mg (0.011 mmol) of dichloropalladium-bistriphenylphosphine, 4 mg (0.021 mmol) of copper (I) iodide, and 0.29 mL (2.08 mmol) of triethylamine. The reaction mixture was heated at 60° C. for 3 h, then allowed to cool to room temperature. The reaction mixture was filtered through a short pad of Celite, diluted with ethyl acetate. The solvents were removed under reduced pressure. The residue was subjected to silica gel chromatography to give the title compound (128 mg, 41%). ¹H-NMR (300 MHz, DMSO-d₆) δ 2.32 (s, 3H), 5.25 (s, 2H), 7.17 (dd, 1H, J=10.2 and 10.2 Hz), 7.25 (d, 2H, J=9.3 Hz), 7.27-7.32 (m, 2H), 7.39-7.49 (m, 3H), 7.57 (d, 2H, J=9.3 Hz), 7.67 (d, 1H, J=7.8 Hz), 7.79 (d, 1H, J=2.1 Hz), 7.89-7.93 (m, 2H), 8.21 (s, 1H), 8.31 (s, 1H), 8.49 (s, 1H), 8.62 (brs, 1H), and 9.09 (s, 1H); ESIMS: 623.2.

b) 6-Ethynyl-1-[(4-methylphenyl)sulfonyl]-1H-indole To a stirred solution of 216 mg (0.588 mmol) of 1-[(4-methylphenyl)sulfonyl]-6-[(trimethylsilyl)ethynyl]-1H-indole, 9 mL of THF, and 1 mL of methanol was added 170 mg (2.94 mmol) of KF. The reaction mixture was allowed to stir at room temperature for 2 h and 162 mg (1.18 mmol) of potassium carbonate was added. After further stirring for 3 h, the solvents were removed under reduced pressure and the residue was passed through a short pad of silica to give the title compound as tan solid (176 mg, 99%). ¹H-NMR (300 MHz, CDCl₃) δ 2.35 (s, 3H), 3.11 (s, 1H), 6.65 (d, 1H, J=3.6 Hz), 7.24 (d, 2H, J=8.4 Hz), 7.34 (d, 1H, J=8.1 Hz), 7.46 (d, 1H, J=8.1 Hz), 7.60 (d, 1H, J=3.6 Hz), 7.77 (d, 2H, J=8.4 Hz), and 8.15 (s, 1H).

c) 1-[(4-Methylphenyl)sulfonyl]-6-[(trimethylsilyl)ethynyl]-1H-indole To a solution containing 300 mg (0.856 mmol) of 6-bromo-1-[(4-methylphenyl)sulfonyl]-1H-indole and 10 mL of dioxane was added 12 mg (0.013 mmol) of tris(dibenylidene-acetone)dipalladium (0), 334 mg (1.03 mmol) of cesium carbonate, 1 mL (7 mmol) of trimethylsilylacetylene, 7 mg (0.034 mmol) of copper (I) iodide, and 6 mg (0.031 mmol) of tri-tert-butylphosphine. The reaction mixture was heated at 80° C. for 13 h and allowed to cool to room temperature. Water was added and the resulting mixture was extracted with dichloromethane. The combined organic layers were dried over Na₂SO₄ and the solvents were removed under reduced pressure. The residue was subjected to silica gel chromatography to give the title compound as a yellow oil (231 mg, 74%). ¹H-NMR (300 MHz, CDCl₃) δ 0.28 (s, 9H), 2.35 (s, 3H), 6.62 (d, 1H, J=3.6 Hz), 7.24 (d, 2H, J=8.4 Hz), 7.32 (dd, 1H, J=8.1 and 4.2 Hz), 7.43 (d, 2H, J=8.4 Hz), 7.57 (d, 1H, J=3.6 Hz), 7.77 (d, 1H, J=8.1 Hz), and 8.14 (s, 1H).

### Example 48

### tert-Butyl-3-{[4-({3-chloro-4-[(3-fluorobenzyl)oxy]phenyl}amino)pyrimidin-5-yl]ethynyl}benzylcarbamate

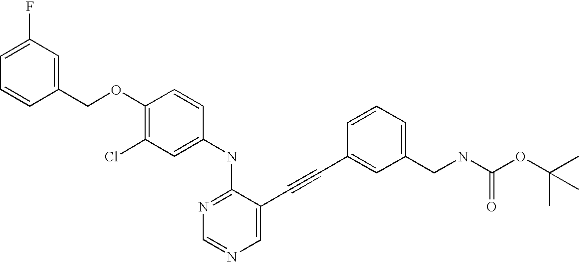

a) In a similar manner as described in Example 24a, from tert-butyl-3-iodobenzylcarbamate (207 mg, 0.62 mmol) was obtained the title compound as an oil (153 mg, 48%). ¹H NMR (300 MHz, DMSO-d₆) δ 1.38 (s, 9H), 4.14 (d, 2H, J=6.9 Hz), 5.24 (s, 2H), 7.17 (dd, 1H, J=8.7 and 8.7 Hz), 7.22 (d, 1H, J=8.7 Hz), 7.27-7.32 (m, 3H), 7.37-7.47 (m, 3H), 7.50-7.56 (m, 2H), 7.74 (d, 1H, J=2.4 Hz), 8.51 (s, 1H), 8.53 (s, 1H), and 9.03 (s, 1H); ESIMS: 559.3.

b) tert-Butyl-3-iodobenzylcarbamate To a stirred solution containing 1 mL (7.5 mmol) of 3-iodobenzylamine and 15 mL of dichloromethane was added 1.7 g (7.87 mmol) of di-tert-butyl dicarbonate. The reaction mixture was allowed to stir at room temperature for 12 h, then diluted with ether and washed successively with water, 10% aqueous NaOH, 10% aqueous HCl, and brine, and dried over MgSO₄. The solvents were removed under reduced pressure to give the title compound as a white solid (2.20 g, 88%). ¹H NMR (300 MHz, CDCl₃) δ 1.45 (s, 9H), 4.26 (d, 2H, J=6.0 Hz), 4.83 (brs, 1H), 7.05 (dd, 1H, J=7.5 and 7.5 Hz), 7.24 (d, 1H, J=7.5 Hz), 7.58 (d, 1H, J=7.5 Hz), and 7.62 (s, 1H).

### Example 49

### N-(3-{[4-({3-Chloro-4-[(3-fluorobenzyl)oxy]phenyl}amino)pyrimidin-5-yl]ethynyl}phenyl)guanidine

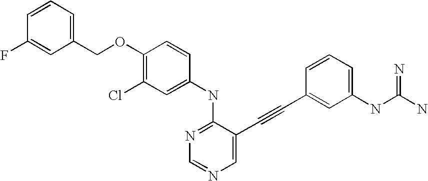

a) In a similar manner as described in Example 24c, from N-(3-ethynylphenyl)guanidine (60 mg, 0.38 mmol) was obtained the title compound as a brown solid (91 mg, 60%). ¹H NMR (300 MHz, DMSO-d₆) δ 5.24 (s, 2H), 5.95 (s, 2H), 7.13-7.54 (m, 10H), 7.75 (d, 1H, J=2.4 Hz), 7.77-7.78 (m, 1H), 8.53 (brs, 1H), 8.76 (s, 1H), and 9.03 (s, 1H); ESIMS: 486.3.

b) N-(3-Ethynylphenyl)guanidine To a stirred solution containing 500 mg (4.27 mmol) of 3-ethynylaniline, 25 mL of ethanol, and 2.5 mL of water was added 1.8 g (42.7 mmol) of NH₂CN and 1.25 mL of concentrated HCl. The reaction mixture was heated at 85° C. for 3 days and allowed to cool to room temperature. The ethanol was removed under reduced pressure and the remainder was partitioned between ether and water. The organic layer was washed with brine and dried over MgSO₄ and the solvents were removed under reduced pressure. The residue was subjected to silica gel chromatography to give the title compound as a white solid (80 mg, 12%). ¹H-NMR (300 MHz, DMSO-d₆) 64.10 (s, 1H), 5.90 (brs, 2H), 6.98 (d, 1H, J=7.2 Hz), 7.21 (dd, 1H, J=7.8 and 7.8 Hz), 7.30 (d, 1H, J=8.1 Hz), 7.62 (s, 1H), and 8.62 (s, 1H); ESIMS: 161.0 (M+H)⁺

### Example 50

### N-(3-{[4-({3-Chloro-4-[(3-fluorobenzyl)oxy]phenyl}amino)pyrimidin-5-yl]ethynyl}benzyl)acetamide

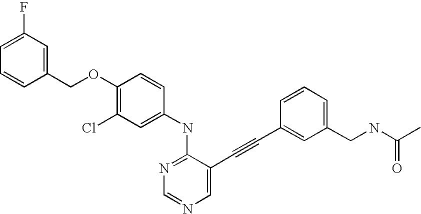

a) To a stirred solution containing 45 mg (0.098 mmol) of 5-{[3-(aminomethyl)phenyl]ethynyl}-N-{3-chloro-4-[(3-fluorobenzyl)oxy]phenyl}pyrimidin-4-amine (Example 50) and 1 mL of DMF was added 11 μL of acetic anhydride and 21 □L of triethylamine. The reaction mixture was allowed to stir at room temperature for 13 h, then diluted with ether, washed with water and brine, and dried over MgSO₄. The solvents were removed under reduced pressure and the residue was subjected to silica gel chromatography to give the title compound as a tan solid (34 mg, 69%). ¹H NMR (300 MHz, CDCl₃) □ 2.02 (s, 3H), 4.42 (d, 2H, J=5.7 Hz), 5.11 (s, 2H), 6.20 (brs, 1H), 6.92 (d, 1H, J=9.0 Hz), 6.99 (dd, 1H, J=9.3 and 9.3 Hz), 7.16-7.21 (m, 2H), 7.29-7.45 (m, 7H), 7.72 (s, 1H), 8.42 (s, 1H), and 8.59 (s, 1H); ESIMS: 501.2.

b) 5-{[3-(Aminomethyl)phenyl]ethynyl}-N-{3-chloro-4-[(3-fluorobenzyl)oxy]-phenyl}pyrimidin-4-amine To a solution containing 123 mg (0.22 mmol) of tert-butyl-3-{[4-({3-chloro-4-[(3-fluorobenzyl)oxy]phenyl}amino)pyrimidin-5-yl]ethynyl}benzyl carbamate (Example 48) and 2 mL of chloroform was added 0.2 mL of trifluoroaceteic acid. The reaction mixture was allowed to stir at room temperature for 2 h, then diluted with chloroform and treated with 1 M NaOH until the pH remained basic. The mixture was extracted with chloroform and the combined organic layers were washed with water and dried over MgSO₄. The solvents were removed under reduced pressure to give the title compound as a tan solid (96 mg, 95%). ¹H NMR (300 MHz, CDCl₃) □ 3.90 (brs, 2H), 5.12 (s, 2H), 6.94 (d, 1H, J=9.0 Hz), 7.00 (dd, 1H, J=7.8 Hz and 7.8 Hz), 7.16-7.22 (m, 3H), 7.30-7.38 (m, 4H), 7.41-7.45 (m, 2H), 7.53 (s, 1H), 7.74 (d, 1H, J=2.4 Hz), 8.47 (s, 1H), and 8.62 (s, 1H); ESIMS: 459.2

### Example 51

### N-{3-Chloro-4-[(3-fluorobenzyl)oxy]phenyl}-5-{[3-({[2-(methylsulfonyl) ethyl]amino}methyl)phenyl]ethynyl}pyrimidin-4-amine

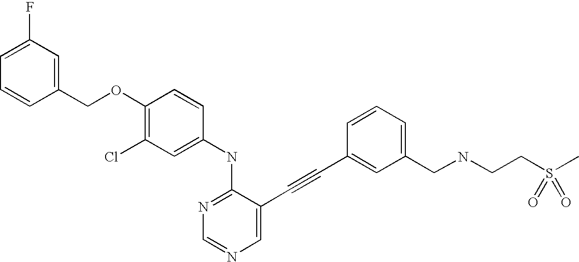

To a solution containing 51 mg (0.11 mmol) of 5-{[3-(aminomethyl)phenyl]ethynyl}-N-{3-chloro-4-[(3-fluorobenzyl)oxy]phenyl}pyrimidin-4-amine (Example 50b) and 1 mL of DMF was added 24 μL (0.266 mmol) of methyl vinyl sulfone and 46 μL (0.334 mmol) of triethylamine. The reaction mixture was allowed to stir at room temperature for 3d, then diluted with ether, washed with water and brine, and dried over MgSO₄. The solvents were removed under reduced pressure and the residue was subjected to silica gel chromatography to give the title compound as a tan solid (21 mg, 34%). ¹H NMR (300 MHz, CDCl₃) δ 2.96 (s, 3H), 3.14-3.20 (m, 4H), 3.83 (s, 2H), 5.13 (s, 2H), 6.95 (d, 1H, J=9.0 Hz), 7.00 (dd, 1H, J=7.5 and 7.5 Hz), 7.17-7.46 (m, 9H), 7.53 (s, 1H), 8.47 (s, 1H), and 8.62 (s, 1H); ESIMS: 565.2.

### Example 52

### 5-{[4-({3-Chloro-4-[(3-fluorobenzyl)oxy]phenyl}amino)pyrimidin-5-yl]ethynyl}-2-furaldehyde

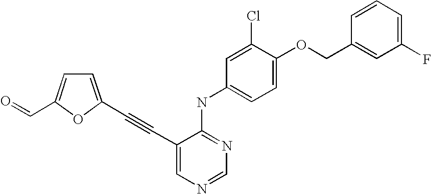

In a similar manner as described in Example 24a, from 5-bromo-2-furaldehyde (192 mg, 1.1 mmol) was obtained the title compound as a tan solid (176 mg, 39%). ¹H NMR (300 MHz, CDCl₃) δ 5.15 (s, 2H), 6.89 (d, 1H, J=3.6 Hz), 6.96 (d, 1H, J=9.0 Hz), 7.01 (dd, 1H, J=8.4 and 8.4 Hz), 7.12 (s, 1H), 7.18-7.25 (m, 2H), 7.29 (d, 1H, J=3.6 Hz), 7.31-7.42 (m, 2H), 7.75 (d, 1H, J=2.4 Hz), 8.53 (brs, 1H), 8.68 (brs, 1H), and 9.67 (s, 1H); ESIMS: 448.0.

### Example 53

### 3-{[(5-{[4-({3-Chloro-4-[(3-fluorobenzyl)oxy]phenyl}amino)pyrimidin-5-yl]ethynyl}-2-furyl)methyl]amino}propanenitrile

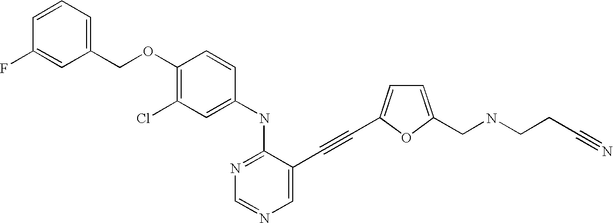

To a solution containing 75 mg (0.17 mmol) of 5-{[4-({3-chloro-4-[(3-fluorobenzyl)oxy]phenyl}amino)pyrimidin-5-yl]ethynyl}-2-furaldehyde (Example 53), 14 μL (0.25 mmol) of acetic acid, 23 μL (0.17 mmol) of triethylamine, and 3 mL of dichloroethane was added 37 μL (0.50 mmol) of 3-aminopropanenitrile. The reaction mixture was heated at 50° C. for 1 h, then cooled to room temperature and 142 mg (0.67 mmol) of sodium cyanoborohydride was added. The reaction mixture was heated at 50° C. for 16 h, then cooled and diluted with dichloromethane. The organic layers were washed with saturated sodium bicarbonate and brine, dried over MgSO₄, and the solvents were removed under reduced pressure. The residue was subjected to silica gel chromatography to give the title compound as a brown solid (42 mg, 50%). ¹H NMR (300 MHz, CDCl₃) δ 2.52 (t, 2H, J=6.3 Hz), 2.95 (t, 2H, J=6.3 Hz), 3.87 (s, 2H), 5.13 (s, 2H), 6.30 (d, 1H, J=3.6 Hz), 6.70 (d, 1H, J=2.1 Hz), 6.94 (d, 1H, J=8.7 Hz), 7.01 (dd, 1H, J=8.1 Hz), 7.17-7.23 (m, 3H), 7.34 (ddd, 1H, J=7.8, 7.8, and 6.0 Hz), 7.42 (dd, 1H, J=8.7 and 2.7 Hz), 7.72 (d, 1H, J=2.7 Hz), 8.46 (s, 1H), and 8.63 (s, 1H); ESIMS: 502.2 (M+H)⁺

### Example 54

### (5-{[4-({3-Chloro-4-[(3-fluorobenzyl)oxy]phenyl}amino)pyrimidin-5-yl]ethynyl}-2-furyl)methanol

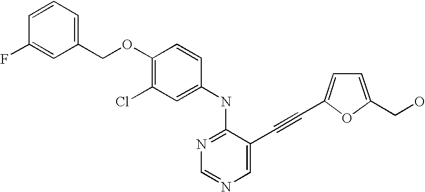

To a solution containing 45 mg (0.10 mmol) of 5-{[4-({3-chloro-4-[(3-fluorobenzyl)oxy]phenyl}amino)pyrimidin-5-yl]ethynyl}-2-furaldehyde (Example 53), 3 mL of methanol, and 1 mL of THF at 0° C. was added 8 mg (0.20 mmol) of sodium borohydride. The reaction mixture was allowed to stir for 50 min, then diluted with hexane and ethyl acetate and passed through a short plug of silica. The solvents were removed under reduced pressure and the residue was subjected to silica gel chromatography to give the title compound as a tan solid (30 mg, 67%). ¹H NMR (300 MHz, CDCl₃) δ4.65 (d, 2H, J=4.8 Hz), 5.15 (s, 2H), 6.38 (d, 1H, J=3.3 Hz), 6.73 (d, 1H, J=3.3 Hz), 6.95 (d, 1H, J=9.0 Hz), 7.01 (dd, 1H, J=9.9 and 9.9 Hz), 7.12-7.28 (m, 3H), 7.32 (d, 1H, J=5.4 Hz), 7.35 (d, 1H, J=5.4 Hz), 7.42 (dd, 1H, J=8.7 and 2.7 Hz), 8.48 (s, 1H), and 8.64 (s, 1H); ESIMS: 450.1.

### Example 55

### (4-{[4-({3-Chloro-4[(3-fluorobenzyl)oxy]phenyl}amino)pyrimidin-5-yl]ethynyl}-1,3-thiazol-2-yl)methanol

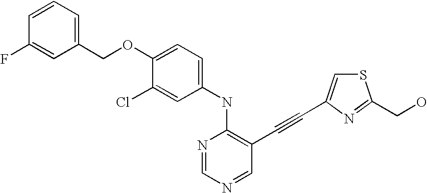

a) In a similar manner as described in Example 24c, from (4-ethynyl-1,3-thiazol-2-yl)methanol (24 mg, 0.11 mmol) was obtained the title compound as a tan solid (13 mg, 19%). ¹H NMR (300 MHz, CDCl₃) δ4.87 (s, 2H), 5.15 (s, 2H), 6.95 (d, 1H, J=9.0 Hz), 7.01 (dd, 1H, J=9.0 and 9.0 Hz), 7.18-7.25 (m, 2H), 7.31-7.58 (m, 2H), 7.62 (d, 1H, J=6.9 Hz), 7.64 (d, 1H, J=13 Hz), 7.74 (d, 1H, J=2.4 Hz), 7.93 (s, 1H), 8.47 (brs, 1H), and 8.62 (s, 1H); ESIMS: 467.1.

b) (4-Ethynyl-1,3-thiazol-2-yl)methanol To a solution containing 44 mg (0.21 mmol) of {4-[(trimethylsilyl)ethynyl]-1,3-thiazol-2-yl}methanol and 5 mL of THF at 0° C. was added 0.42 mL (0.42 mmol) of 1.0M TBAF in THF. The reaction mixture was allowed to stir for 20 min, then quenched by the addition of water and extracted with ethyl acetate. The organic layers were washed with water and brine and dried over MgSO₄. The solvents were removed under reduced pressure and the residue was passed over a short pad of silica gel to give the title compound as a brown solid (24 mg, 83%). ¹H NMR (300 MHz, CDCl₃) δ3.10 (s, 1H), 3.20 (brs, 1H), 4.93 (s, 2H), and 7.48 (s, 1H).

c) {4-[(Trimethylsilyl)ethynyl]-1,3-thiazol-2-yl}methanol To a solution containing 56 mg (0.29 mmol) of (4-bromo-1,3-thiazol-2-yl)methanol, 120 μL (0.86 mmol) of trimethylsilylacetylene, and 5 mL of THF was added 8 mg (0.012 mmol) of dichloropalladiumbistriphenylphosphine, 1 mg (0.006 mmol) of copper (I) iodide, and 0.17 mL (1.2 mmol) of triethylamine. The reaction mixture was heated at 60° C. for 13 h, then allowed to cool to room temperature. The reaction mixture was filtered through a short pad of Celite, diluting with ethyl acetate, and the solvents were removed under reduced pressure. The residue was subjected to silica gel chromatography to give the title compound as a tan oil (44 mg, 72%). ¹H NMR (300 MHz, CDCl₃) δ 0.24 (s, 9H), 2.43 (t, 1H, J=6.0 Hz), 4.93 (d, 2H, J=6.0 Hz), and 7.45 (s, 1H); ESIMS: 211.9.

d) (4-Bromo-1,3-thiazol-2-yl)methanol To a solution containing 0.5 mL (1.1 mmol) of 2.3M n-butyllithium in hexane and 2 mL of THF at −78° C. was added a solution containing 243 mg (1.0 mmol) of 2,4-dibromo-1,3-thiazole and 5 mL of THF slowly. The bright yellow reaction mixture was allowed to stir for 15 min and 0.15 μL of DMF was added. The orange reaction mixture was allowed to stir for a further 30 min and quenched by the addition of methanol. To this crude reaction mixture was added 63 μL of acetic acid and 34 mg (1.0 mmol) of sodium borohydride. The mixture was allowed to warm slowly to room temperature, then quenched by the addition of water and extracted with ethyl acetate. The organic layers were washed with water and brine, then dried over MgSO₄. The solvents were removed under reduced pressure and the residue was subjected to silica gel chromatography to give the title compound (66 mg, 34%). ¹H NMR (300 MHz, CDCl₃) δ 4.00 (brs, 1H), 4.93 (s, 3H), and 7.20 (s, 1H).

### Example 56

### N-{3-Chloro-4-[(3-fluorobenzyl)oxy]phenyl}-5-(1,2,3,4-tetrahydro-isoquinolin-7-ylethynyl)pyrimidin-4-amine

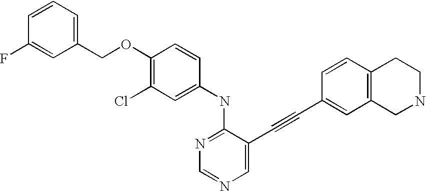

a) In a similar manner as described in Example 24a, from 7-iodo-1,2,3,4-tetrahydroisoquinoline hydrochloride (100 mg, 0.34 mmol) was obtained the title compound as tan solid (55 mg, 40%). ¹H NMR (300 MHz, CDCl₃) δ 2.86 (brs, 1H), 3.25 (brs, 2H), 4.07-4.17 (brm, 2H), 4.80 (brs, 2H), 5.12 (s, 2H), 6.87 (d, 1H, J=8.1 Hz), 6.93 (d, 1H, J=9.0 Hz), 7.00 (dd, 1H, J=9.0 and 9.0 Hz), 7.12-7.48 (m, 7H), 7.73 (d, 1H, J=2.4 Hz), 8.46 (brs, 1H), and 8.62 (brs, 1H); ESI MS: 485.2 (M+H)⁺

b) 7-Iodo-1,2,3,4-tetrahydroisoquinoline hydrochloride To a solution containing 0.48 g (1.6 mmol) of 2-acetyl-7-iodo-1,2,3,4-tetrahydroisoquinoline, 15 mL of ethanol, and 2 mL of water was added 2 mL of concentrated HCl. The reaction mixture was heated at reflux for 18 h, then cooled to room temperature and the ethanol was removed under reduced pressure. The aqueous mixture was treated with 10% aqueous sodium hydroxide until basic and extracted with ethyl acetate. The organic layers were washed with water and brine and dried over MgSO₄. The solvents were removed under reduced pressure and the residue was taken up in methanol, triturated with concentrated HCl, and filtered to give the title compound as a brown solid (460 mg, 97%), that was used without further purification. ¹H NMR (300 MHz, CDCl₃) δ 1.77 (brs, 1H), 2.69 (t, 2H, J=6.0 Hz), 3.07 (t, 2H, J=6.0 Hz), 3.91 (s, 2H), 6.79 (d, 1H, J=8.1 Hz), 7.32 (s, 1H), and 7.40 (d, 1H, J=8.4 Hz); ESIMS: 259.8.

c) 2-Acetyl-7-iodo-1,2,3,4-tetrahydroisoquinoline To a solution containing 0.60 g (3.15 mmol) of 2-acetyl-1,2,3,4-tetrahydroisoquinolin-7-amine, 1.1 mL of concentrated HCl, and 4.75 mL of water at 0° C. was added 0.33 g (4.73 mmol) of sodium nitrite. The reaction mixture was allowed to stir for 30 min, then added to a stirred solution containing 1.05 g (6.3 mmol) of potassium iodide, 3.75 mL of water, 42 mg of copper (I) iodide, and 12.5 mL of chloroform. The combined reaction mixture was allowed to warm to room temperature, stirred for 13 h, and diluted with dichloromethane. The organic layers were washed with aqueous sodium bisulfite and brine and dried over Na₂SO₄. The solvents were removed under reduced pressure to give the title compound as a tan solid (820 mg, 86%). ¹H NMR (300 MHz, CDCl₃) δ 2.16 (s, 3H), 2.77 (t, 0.8H, J=6.0 Hz), 2.84 (t, 1.2H, J=6.0 Hz), 3.65 (t, 1.2H, J=6.0 Hz), 3.79 (t, 0.8H, J=6.0 Hz), 4.55 (s, 0.8H), 4.67 (s, 1.2H), 6.89 (dd, 1H, J=8.1 Hz), and 7.45-7.52 (m, 2H); ESIMS: 301.4.

d) 2-Acetyl-1,2,3,4-tetrahydroisoquinolin-7-amine To a solution containing 0.70 g (3.2 mmol) of 2-acetyl-7-nitro-1,2,3,4-tetrahydroisoquinoline and 5 mL of ethanol was added 0.5 mL of concentrated HCl and 29 mg of platinum oxide. The reaction mixture was subjected to an atmosphere of hydrogen at 40 psi until hydrogen uptake had ceased. The crude reaction mixture was filtered through a short pad of Celite and the solvents were removed under reduced pressure. The residue was taken up in water, made basic by the addition of 10% aqueous sodium hydroxide, and extracted with dichloromethane and dried over MgSO₄. The solvents were removed under reduced pressure to give the title compound as a brown solid (600 mg, 100%). ¹H NMR (300 MHz, CDCl₃) δ 2.15 (s, 3H), 2.71 (t, 0.9H, J=6.0 Hz), 2.78 (t, 1.1H, J=6.0 Hz), 3.59 (brs, 2H), 3.62 (t, 1.1H, J=6.0 Hz), 3.77 (t, 0.9H, J=6.0 Hz), 4.50 (s, 1.1H), 4.62 (s, 0.9H), 6.46 (d, 1H, J=14.1 Hz), 6.53 (ddd, 1H, J=8.7, 8.7, and 2.1 Hz), and 6.93 (dd, 1H, J=10.5 Hz); ESIMS: 191.4 (M+H)⁺

e) 2-Acetyl-7-nitro-1,2,3,4-tetrahydroisoquinoline To a slurry containing 710 mg (3.3 mmol) of 7-nitro-1,2,3,4-tetrahydroisoquinoline and 10 mL of THF at 0° C. was added 1.15 mL (8.3 mmol) of triethylamine, followed by 0.25 mL (3.5 mmol) of acetyl chloride. The reaction mixture was allowed to stir for 30 min, then diluted with ethyl acetate, washed with water and brine, and dried over MgSO₄. The solvents were removed under reduced pressure to give the title compound as a brown oil (700 mg, 96%). ¹H NMR (300 MHz, CDCl₃) δ2.19 (s, 1.8H), 2.20 (s, 1.2H), 2.94 (t, 0.8H, J=5.7 Hz), 3.00 (t, 1.2H, J=5.7 Hz), 3.72 (t, 0.8H, J=6.0 Hz), 3.86 (t, 1.2H, J=6.0 Hz), 4.71 (s, 0.8H), 4.82 (s, 1.2H), 7.32 (dd, 1H, J=8.4 Hz), and 8.01-8.07 (m, 2H); ESIMS: 221.0 (M+H)⁺

### Example 57

### 2-{[4-({3-Chloro-4-[(3-fluorobenzyl)oxy]phenyl}amino)pyrimidin-5-yl]ethynyl}benzaldehyde

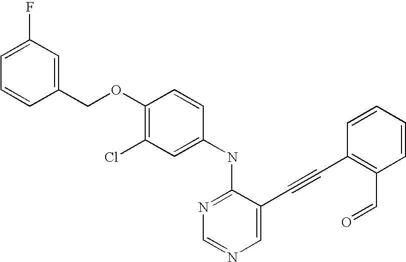

In a similar manner as described in Example 24a, from 2-iodobenzaldehyde (72 mg, 0.31 mmol) was obtained the title compound as a tan solid (48 mg, 37%). ¹H NMR (300 MHz, DMSO-d₆) δ 5.25 (s, 2H), 7.17 (dd, 1H, J=9.0 and 9.0 Hz), 7.28-7.33 (m, 2H), 7.46 (ddd, 1H, J=8.1, 8.1, and 6.6 Hz), 7.65 (d, 1H, J=9.3 Hz), 7.70 (d, 1H, J=7.8 Hz), 7.79 (dd, 1H, J=7.5 and 7.5 Hz), 7.85 (dd, 1H, J=7.8 Hz), 7.91 (d, 1H, J=2.7 Hz), 8.02 (d, 1H, J=7.2 Hz), 8.62 (d, 1H, J=3.0 Hz), 9.17 (s, 1H), and 10.34 (s, 1H); ESIMS: 458.1.

### Example 58

### N-{3-Chloro-4-[(3-fluorobenzyl)oxy]phenyl)-5-([5-({[2-(methylsulfonyl)ethyl]amino}methyl)-2-furyl]ethynyl}pyrimidin-4-amine

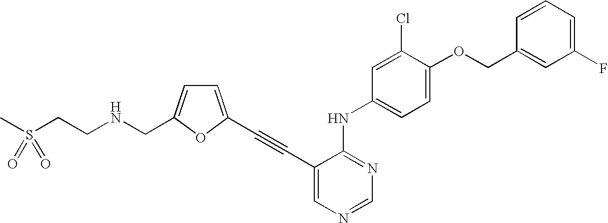

a) In a similar manner as described in Example 24a, from N-[(5-ethynyl-2-furyl)methyl]-2-(methylsulfonyl)ethanamine (50 mg, 0.22 mmol) was obtained the title compound as a beige solid (21 mg, 17%). ¹H NMR (400 MHz, CDCl₃) δ 2.99 (s, 3H), 3.18 (s, 4H), 3.85 (s, 2H), 5.15 (s, 2H), 6.30 (d, J=3 Hz, 1H), 6.72 (d, J=3 Hz, 1H), 6.95 (d, J=9 Hz, 2H), 7.18-7.23 (m, 2H), 7.27-7.38 (m, 1H), 7.43-7.46 (m, 1H), 7.74 (d, J=3 Hz, 1H), 8.47 (s, 1H), 8.64 (s, 1H).

b) N-[(5-Ethynyl-2-furyl)methyl]-2-(methylsulfonyl)ethanamine A mixture of N-[(5-bromo-2-furyl)methyl]-2-(methylsulfonyl)ethanamine (1.2 g, 4.25 mmol), dichlorobis(triphenylphosphine)palladium(II) (35 mg, 0.085 mmol), ethynyl(trimethyl)silane (0.46 g, 4.68 mmol), copper(I) iodide (32.4 mg, 0.17 mmol) and triethylamine (1.2 mL, 8.5 mmol) in tetrahydrofuran (5 mL) was stirred at rt under nitrogen for 48 h. The mixture was diluted with ethyl acetate, washed with water, with brine, dried and concentrated. The residue in dichloromethane (5 mL) under stirring and nitrogen was cooled to 0° C. and treated with 1 M tetrabutylammonium fluoride in tetrahydrofuran (5 mL). The mixture was let warm to rt and stirred at rt for 30 min, diluted with dichloromethane, washed with water, with brine, dried and concentrated. Purification was accomplished on silica gel eluting with ethyl acetate to give the title compound as a yellow oil (0.32 g, 35%). ¹H NMR (400 MHz, CDCl₃) δ 2.99 (s, 3H), 3.13 (s, 4H), 3.39 (s, 1H), 3.78 (s, 2H), 6.18 (d, J=3 Hz, 1H), 6.58 (d, J=3 Hz, 1H).

c) N-[(5-Bromo-2-furyl)methyl]-2-(methylsulfonyl)ethanamine A mixture of 2-(methylsulfonyl)ethanamine (30 g, 0.24 mol) and 5-bromo-2-furaldehyde (28.4 g, 0.16 mol) in ethanol:tetrahydrofuran 1:1 (300 mL) was stirred for 1 h at rt under nitrogen before sodium borohydride (30 g, 0.79 mol) was added portionwise. The resulting mixture was stirred overnight at rt under nitrogen. Saturated sodium carbonate solution was added and the mixture was extracted with ethyl acetate. The organic layer was washed with brine, dried and concentrated to give the title compound as a colorless oil (33 g, 72%). ¹H NMR (400 MHz, DMSO-d₆) δ 2.86 (t, J=6 Hz, 2H), 2.98 (s, 3H), 3.20 (t, J=6 Hz, 2H), 3.66 (s, 2H), 6.31 (d, J=3 Hz, 1H), 6.46 (d, J=3 Hz, 1H).

### Example 59

### N-(3-{[4-({3-Chloro-4-[(3-fluorobenzyl)oxy]phenyl}amino)pyrimidin-5-yl]ethynyl}phenyl)-2-(2-methoxyethoxy)acetamide

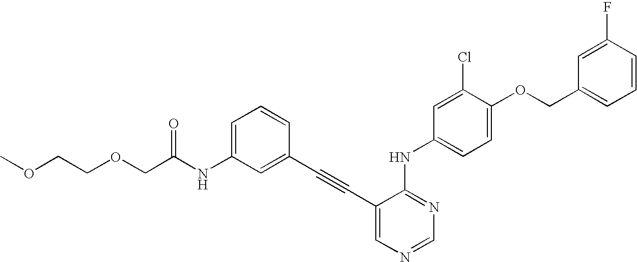

In a similar manner as described in Example 24c, from N-(3-ethynylphenyl)-2-(2-methoxyethoxy)acetamide (38 mg, 0.24 mmol) was obtained the title compound as a yellow solid (66 mg, 53%). ¹H NMR (400 MHz, CDCl₃) δ 3.47 (s, 3H), 3.62 (m, 2H), 3.76 (m, 2H), 4.11 (s, 2H), 5.12 (s, 2H), 6.93 (d, J=9 Hz, 1H), 7.00 (m, 1H), 7.18-7.37 (m, 6H), 7.43 (dd, J=3.9 Hz, 1H), 7.53 (d, J=8 Hz, 1H), 7.76 (d, J=3 Hz, 1H), 7.95 (s, 1H), 8.47 (s, 1H), 8.63 (s, 1H), 8.99 (s, 1H); ESIMS: 561 (M+H)⁺

### Example 60

### N-[3-({4-[(2-Benzyl-1H-benzimidazol-5-yl)amino]pyrimidin-5-yl}ethynyl)phenyl]acetamide

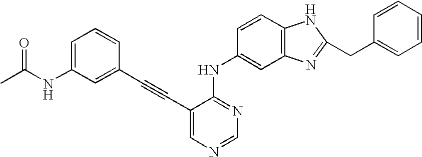

In a similar manner as described in Example 24c, from N-(3-ethynylphenyl)acetamide (82 mg, 0.51 mmol) and 2-benzyl-N-(5-iodopyrimidin-4-yl)-1H-benzimidazol-5-amine was obtained the title compound as a yellow solid (34 mg, 16%). ¹H NMR (400 MHz, DMSO-d₆) δ 2.04 (s, 3H), 4.15 (s, 2H), 7.19-7.33 (m, 8H), 7.40 (m, 1H), 7.54 (m, 1H), 7.72 (s, 1H), 7.91 (s, 1H), 8.46 (s, 1H), 8.47 (d, J=9 Hz, 1H), 9.05 (s, 1H), 10.05 (s, 1H), 12.30 (br s, 1H); ESIMS: 459 (M+H)⁺

### Example 61

### N¹-(3-([4-({3-Chloro-4-[(3-fluorobenzyl)oxy]phenyl}amino)pyrimidin-5-yl]ethynyl)phenyl)-β-alaninamide

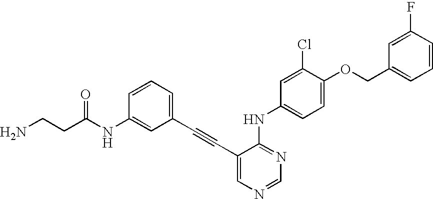

In a similar manner as described in Example 24c, from ethynylaniline (424 mg, 3.6 mmol) was obtained the cross-coupled aniline product. A mixture of this intermediate (50 mg, 0.11 mmol), N-(tert-butoxycarbonyl)-β-alanine (21 mg, 0.11 mmol), 1-(3-dimethylaminopropyl)-3-ethylcarbodiimide hydrochloride (25 mg, 0.13 mmol) and 1-hydroxybenzotriazole hydrate (14 mg, 0.11 mmol) in dimethylformamide (1 mL) was stirred at rt under nitrogen for 16 h. The reaction mixture was concentrated, diluted with ethyl acetate, washed with water, with brine, dried and concentrated. The residue was dissolved in dichloromethane (1.5 mL), treated with trifluoroacetic acid (0.4 mL) and the mixture was stirred for 30 min at rt. The reaction mixture was concentrated, redissolved in ethyl acetate, washed with 10% aqueous sodium carbonate solution, with brine, dried and concentrated. Trituration with hexane gave the title compound as a light pink solid (30 mg, 53%). ¹H NMR (400 MHz, DMSO-d₆) δ 2.66 (t, J=6 Hz, 2H), 3.05 (t, J=6 Hz, 2H), 5.23 (s, 2H), 7.13-7.56 (m, 11H), 7.72 (s, 1H), 7.93 (s, 1H), 8.52 (d, J=9 Hz, 2H), 9.02 (s, 1H), 10.27 (br s, 1H); ESIMS: 516 (M+H)⁺

### Example 62

### N-(3-{[4-({3-Chloro-4-[(3-fluorobenzyl)oxy]phenyl}amino)pyrimidin-5-yl]ethynyl}phenyl)-2-(methylsulfonyl)acetamide

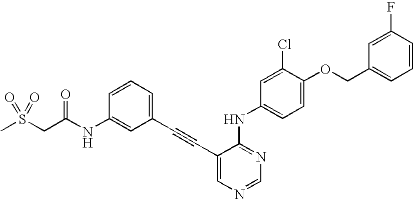

A mixture of 5-[(3-aminophenyl)ethynyl]-N-{3-chloro-4-[(3-fluorobenzyl)oxy]phenyl}pyrimidin-4-amine (50 mg, 0.11 mmol) (Example 63), (methylsulfonyl)acetic acid (17 mg, 0.12 mmol), 1-(3-dimethylaminopropyl)-3-ethylcarbodiimide hydrochloride (25 mg, 0.13 mmol) and 1-hydroxybenzotriazole hydrate (14 mg, 0.11 mmol) in dimethylformamide (1 mL) was stirred at rt under nitrogen for 16 h. The reaction mixture was concentrated, diluted with ethyl acetate, washed with water, with brine, dried and concentrated. Chromatography on silica gel eluting with ethyl acetate gave the title compound as a white solid (40 mg, 64%). ¹H NMR (400 MHz, DMSO-d₆) δ 3.16 (s, 3H), 4.28 (s, 2H), 5.23 (s, 2H), 7.13-7.55 (m, 9H), 7.74 (d, J=3 Hz, 1H), 7.95 (s, 1H), 8.52 (s, 2H), 9.06 (s, 1H), 10.55 (s, 1H); ESIMS: 565 (M+H)⁺

### Example 63

### N-[3-({4-[(4-Benzylphenyl)amino]pyrimidin-5-yl}ethynyl)phenyl]-acetamide

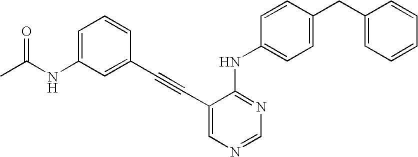

In a similar manner as described in Example 24c, from N-(3-ethynylphenyl)acetamide (56 mg, 0.35 mmol) and 5-iodo-N-[4-(phenylmethyl)phenyl]-4-pyrimidinamine (124 mg, 0.32 mmol) was obtained the title compound as a white solid (32 mg, 24%). ¹H NMR (400 MHz, DMSO-d₆) δ 2.19 (s, 3H), 3.97 (s, 2H), 7.18-7.36 (m, 11H), 7.47 (d, J=8 Hz, 1H), 7.55 (d, J=8 Hz, 2H), 7.81 (s, 1H), 8.47 (s, 1H), 8.63 (s, 1H); ESIMS: 419 (M+H)⁺

### Example 64

### N-[3-({4-[(4-Phenoxyphenyl)amino]pyrimidin-5-yl}ethynyl)phenyl]acetamide

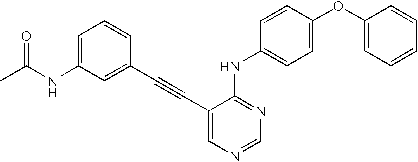

In a similar manner as described in Example 24c, from N-(3-ethynylphenyl)acetamide (39 mg, 0.24 mmol) and 5-iodo-N-(4-phenoxyphenyl)pyrimidin-4-amine (83 mg, 0.21 mmol) was obtained the title compound as a light yellow solid (45 mg, 51%). ¹H NMR (400 MHz, DMSO-d₆) δ 2.17 (s, 3H), 7.01-7.10 (m, 5H), 7.25-7.37 (m, 5H), 7.46 (d, J=8 Hz, 1H), 7.58 (d, J=9 Hz, 2H), 7.73 (s, 1H), 7.86 (s, 1H), 8.45 (s, 1H), 8.62 (s, 1H). ESIMS: 421 (M+H)⁺

### Example 65

### N-[3-({4-[(1-Benzyl-1H-indazol-5-yl)amino]pyrimidin-5-yl}ethynyl)phenyl]acetamide

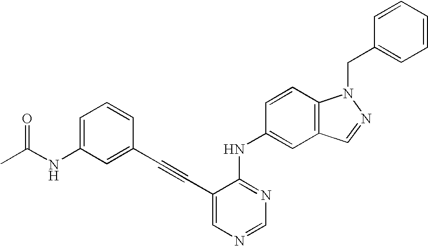

In a similar manner as described in Example 24c, from N-(3-ethynylphenyl)acetamide (38 mg, 0.23 mmol) and 1-benzyl-N-(5-iodopyrimidin-4-yl)-1H-indazol-5-amine (100 mg, 0.23 mmol) was obtained the title compound as a white solid (48 mg, 46%). ¹H NMR (400 MHz, DMSO-d₆) δ 2.04 (s, 3H), 5.65 (s, 2H), 7.19-7.37 (m, 7H), 7.48-7.55 (m, 2H), 7.67 (d, J=9 Hz, 1H), 7.92 (s, 2H), 8.09 (s, 1H), 8.48 (d, J=112 Hz, 2H), 9.13 (s, 1H), 10.06 (s, 1H); ESIMS: 459 (M+H)⁺

### Example 66

### 1-Benzyl-N-[5-(phenylethynyl)pyrimidin-4-yl]-1H-indol-5-amine

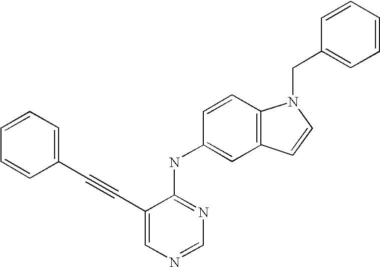

In a similar manner as described in Example 24c, from phenyl acetylene (0.02 mL, 0.18 mmol) and N-(5-iodo-4-pyrimidinyl)-1-(phenylmethyl)-1H-indol-5-amine (100 mg, 0.412 mmol) was obtained the title compound as a brown solid. ¹H NMR (300 MHz, Acetone-d₆) δ5.44 (s, 2H), 6.51-6.50 (dd, J=0.73, 3.11 Hz, 1H), 7.44-7.19 (m, 11H), 7.65-7.62 (m, 2H), 7.91 (s, 1H), 8.33 (s, 1H), 8.47-8.44 (d, J=11.7 Hz, 2H).

### Example 67

### 5-[(6-Amino-2-pyridinyl)ethynyl]-N-(3-chloro-4-{[(3-fluorophenyl) methyl]oxy}phenyl)₄-pyrimidinamine

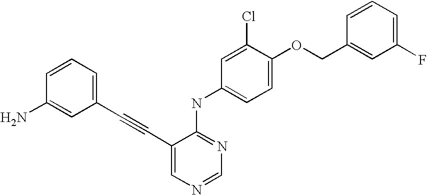

a) In a similar manner as described in Example 24c, from 6-ethynyl-2-pyridinamine (607 mg, 5.1 mmol), was obtained the title compound as a beige solid (1.1 g, 58%). ¹H NMR (300 MHz, CDCl₃) δ 5.14 (s, 1H), 5.18 (s, 2H), 6.64 (d, J=8.4 Hz, 1H), 6.97-7.08 (m, 3H), 7.25 (m, 2H), 7.39 (m, 1H), 7.56 (m, 2H), 7.83 (d, J=2.6 Hz, 1H), 7.88 (s, 1H), 8.54 (s, 1H), 8.68 (s, 1H); ESIMS: 446 (M+H)⁺

b) 6-Ethynyl-2-pyridinamine 6-bromo-2-pyridinamine (1.50 g, 8.7 mmol), TMS acetylene (2.45 mL, 17.4 mmol), Pd(PPh₃)₂Cl₂ (304 mg, 0.4 mmol), CuI (165 mg, 0.9 mmol), and TEA (3.6 mL, 26 mmol) were added to THF (40 mL) at RT. The mixture was heated at 50° C. for 1 h. The mixture was cooled and then water (100 mL) was added and the mixture extracted with ethyl acetate (2×200 mL). The combined organic layers were washed with brine, dried with MgSO₄, filtered, and evacuated in vacuo. The residue was passed through a pad of silica gel (50% EA/Hexanes) to provide the silyl protected intermediate. The dark solid was dissolved in THF (50 mL) and TBAF (1.0 M in THF, 17 mL, 17 mmol) was added dropwise at RT. The solution was stirred 30 min. at RT. Water (100 mL) was added and the mixture extracted with ethyl acetate (2×200 mL). The combined organic layers were washed with brine, dried with MgSO₄, filtered, and evacuated in vacuo. The residue was purified by silica gel column chromatography (1% MeOH/CH₂Cl₂) to provide the title compound as a brown solid (890 mg, 87%).

### Example 68

### N-{6-[2-(4-{3-Chloro-4-[(3-fluorobenzyl)oxy]anilino}-5-pyrimidinyl)ethynyl]-2-pyridinyl}acetamide

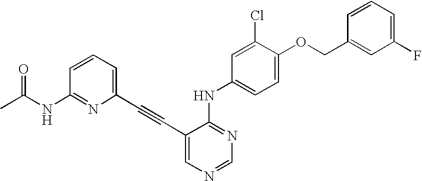

Acetic anhydride (0.014 mL, 0.1 mmol) was added to 5-[(6-amino-2-pyridinyl)ethynyl]-N-(3-chloro-4-{[(3-fluorophenyl)methyl]oxy}phenyl)-4-pyrimidine amine (43 mg, 0.1 mmol) (Example 69) in CH₂Cl₂ (1 mL) and was heated to reflux overnight. The mixture was cooled and then saturated aqueous NaHCO₃ (50 mL) was added and the mixture extracted with ethyl acetate (2×150 mL). The combined organic layers were washed with brine, dried with MgSO₄, filtered, and evacuated in vacuo. The residue was triturated (EA/hexanes) to provide the title compound as a beige solid (33 mg, 70%). ¹H NMR (400 MHz, DMSO-d₆) δ2.07 (s, 3H), 5.23 (s, 2H), 7.15 (dt, J=8.6, 2.4 Hz, 1H), 7.21 (d, J=9.0 Hz, 1H), 7.29 (m, 2H), 7.45 (m, 1H), 7.50 (dd, J=8.8, 2.5 Hz, 1H), 7.52 (d, J=7.1 Hz, 1H), 7.74 (d, J=2.6 Hz, 1H), 7.84 (t, J=8.1 Hz, 1H), 8.10 (d, J=8.4 Hz, 1H), 8.50 (s, 1H), 8.56 (s, 1H), 9.08 (s, 1H), 10.76 (s, 1H); ESIMS: 487 (M+H)⁺

### Example 69

### 2-Chloro-N-{6-[(4-{3-chloro-4-[(3-fluorobenzyl)oxy]anilino}-5-pyrimidinyl)ethynyl]-2-pyridinyl}-2,2-difluoroacetamide

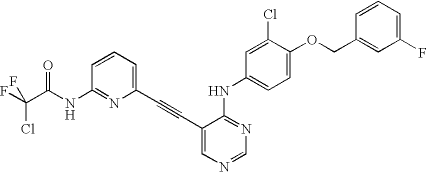

Chlorodifluoroacetic anhydride (0.022 mL, 0.1 mmol) was added to 5-[(6-amino-2-pyridinyl)ethynyl]-N-(3-chloro-4-{[(3-fluorophenyl)methyl]oxy}phenyl)-4-pyrimidinamine (37 mg, 0.1 mmol) (Example 69) in CH₂Cl₂ (1 mL) and was heated to reflux overnight. The mixture was cooled and then saturated aqueous NaHCO₃ (50 mL) was added and the mixture extracted with ethyl acetate (2×150 mL). The combined organic layers were washed with brine, dried with MgSO₄, filtered, and evacuated in vacuo. The residue was triturated (EA/hexanes) to provide the title compound as a brown solid (26 mg, 57%). ¹H NMR (300 MHz, CDCl₃) δ 5.20 (s, 2H), 7.02 (d, J=8.9 Hz, 1H), 7.07 (t, J=7.0 Hz, 1H), 7.22-7.25 (m, 2H), 7.37-7.53 (m, 3H), 7.74 (s, 1H), 7.76 (d, J=4.4 Hz, 1H), 7.93 (t, J=8.0 Hz, 1H), 8.31 (d, J=8.4 Hz, 1H), 8.60 (bs, 1H), 8.73 (bs, 1H); ESIMS: 558 (M+H)⁺

### Example 70

### N-{6-[(4-{3-Chloro-4-[(3-fluorobenzyl)oxy]anilino}-5-pyrimidinyl)ethynyl]-2-pyridinyl}-4-(dimethylamino)butanamide

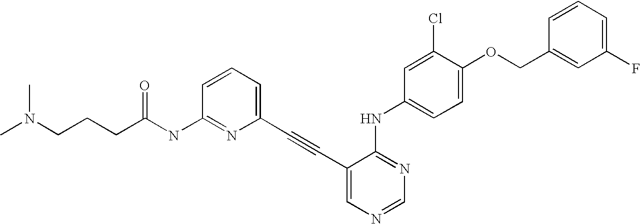

1,3-Dicyclohexylcarbodiimide (25 mg, 0.1 mmol) was added to a RT mixture of 5-[(6-amino-2-pyridinyl)ethynyl]-N-(3-chloro-4-{[(3-fluorophenyl)methyl]oxy}phenyl)-4-pyrimidinamine (53 mg, 0.1 mmol) (Example 69), TEA (0.017 ml, 0.1 mmol) and DMAP (2 mg) in 1,2-Dichloroethane (1 mL). The mixture was heated at 55° C. overnight. The mixture was cooled and then passed through a celite packed glass frit, which was rinsed with CH₂Cl₂. The resulting organic eluant was concentrated and purified by silica gel column chromatography (5-15% MeOH/CH₂Cl₂) to give an oily solid. The residue was triturated with EtOAc/CH₂Cl₂/Et₂O and filtered to give the title compound as a brown powder (22 mg, 31%). ¹H NMR (300 MHz, DMSO-d₆) δ 5.28 (s, 2H), 7.17-7.35 (m, 4H), 7.45-7.57 (m, 2H), 7.59 (d, J=7.4 Hz, 1H), 7.78 (d, J=2.5 Hz, 1H), 7.90 (t, J=8.0 Hz, 1H), 8.16 (d, J=8.4 Hz, 1H), 8.55 (bs, 1H), 8.61 (s, 1H), 9.15 (s, 1H), 10.89 (s, 1H); ESIMS: 559 (M+H)⁺

### Example 71

### Methyl 4-({6-[(4-{3-chloro-4-[(3-fluorobenzyl)oxy]anilino}-5-pyrimidinyl)ethynyl]-2-pyridinyl}amino)-4-oxobutanoate

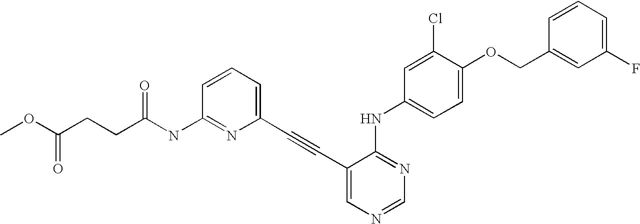

Methyl 4-chloro-4-oxobutanoate (0.017 mL, 0.1 mmol) was added to a solution 5-[(6-amino-2-pyridinyl)ethynyl]-N-(3-chloro-4-{[(3-fluorophenyl)methyl]oxy}phenyl)-4-pyrimidinamine (42 mg, 0.1 mmol) (Example 69) and DIEA (0.05 mL, 0.3 mmol) in CH₂Cl₂ (1 mL) and was heated to reflux overnight. The mixture was cooled and then saturated aqueous NaHCO₃ (50 mL) was added and the mixture extracted with ethyl acetate (2×150 mL). The combined organic layers were washed with brine, dried with MgSO₄, filtered, and evacuated in vacuo. The residue was triturated (EA/hexanes) to provide the title compound as a beige solid (27 mg, 51%). ¹H NMR (300 MHz, CDCl₃) δ 1.94 (bs, 4H), 3.75 (s, 3H), 5.24 (s, 2H), 6.99 (d, J=7.4 Hz, 1H), 7.06-7.43 (m, 8H), 7.54 (d, J=2.3 Hz, 1H), 7.70 (t, J=8.0 Hz, 1H), 8.24 (d, J=8.2 Hz, 1H), 8.99 (s, 1H), 9.18 (s, 1H); ESIMS: 559 (M+H)⁺

### Example 72

### N-{3-Chloro-4-[(3-fluorobenzyl)oxy]phenyl}-5-[(6-{[2-(methylsulfonyl)ethyl]amino}-2-pyridinyl)ethynyl]-4-pyrimidinamine

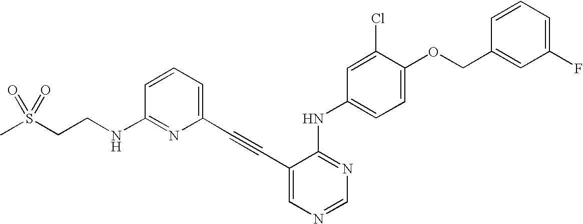

Sodium hydride (60% dispersion, 6 mg, 0.2 mmol) was added to 5-[(6-amino-2-pyridinyl)ethynyl]-N-(3-chloro-4-{[(3-fluorophenyl)methyl]oxy}phenyl)-4-pyrimidine amine (50 mg, 0.1 mmol) (Example 69) in DMF (1 mL) and stirred for 10 min. at RT. Methyl vinyl sulfone (0.013 mL, 0.1 mmol) was added and the mixture was stirred overnight. MeOH (5 mL) was added to the mixture and it was concentrated in vacuo. The residue was purified by silica gel column chromatography (5% MeOH/CH₂Cl₂) to provide the title compound as a brown powder (37 mg, 59%). ¹H NMR (300 MHz, DMSO-d₆) δ 2.93 (s, 3H), 3.02 (m, 2H), 3.28 (m, 2H), 5.32 (s, 2H), 6.49 (d, J=8.3 Hz, 1H), 7.20 (m, 2H), 7.33 (m, 2H), 7.36 (m, 3H), 7.51 (m, 3H), 8.80 (s, 1H), 9.14 (s, 1H); ESIMS: 552 (M+H)⁺

### Example 73

### {6-[(4-{3-Chloro-4-[(3-fluorobenzyl)oxy]anilino}-5-pyrimidinyl)ethynyl]-2-pyridinyl}methanol

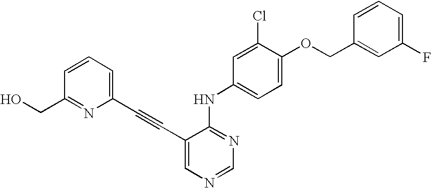

a) In a similar manner as described in Example 24c, from (6-ethynyl-2-pyridinyl)methanol (700 mg, 5.3 mmol) was obtained the title compound as light brown powder (1.54 g, 76%). ¹H NMR (300 MHz, DMSO-d₆) δ 4.60 (d, J=5.5 Hz, 2H), 5.27 (s, 2H), 5.55 (t, J=5.5 Hz, 1H), 7.17-7.35 (m, 4H), 7.45-7.57 (m, 3H), 7.72 (d, J=7.7 Hz, 1H), 7.78 (d, J=2.5 Hz, 1H), 7.92 (t, J=7.8 Hz, 1H), 8.60 (s, 2H), 9.14 (s, 1H); ESIMS: 461 (M+H)⁺

b) (6-Ethynyl-2-pyridinyl)methanol (6-Bromo-2-pyridinyl)methanol (1.51 g, 8.0 mmol), TMS acetylene (2.27 mL, 16.0 mmol), Pd(PPh₃)₂Cl₂ (282 mg, 0.4 mmol), CuI (153 mg, 0.8 mmol), and TEA (3.4 mL, 24 mmol) were added to THF (40 mL) at RT. The mixture was heated at 50° C. for 1 h. The mixture was cooled and then water (100 mL) was added and the mixture extracted with ethyl acetate (2×150 mL). The combined organic layers were washed with brine, dried with MgSO₄, filtered, and evacuated in vacuo. The residue was purified by silica gel column chromatography (30% EA/Hexanes) to provide the silyl protected intermediate. The oil was dissolved in THF (50 mL) and TBAF (1.0 M in THF, 16 mL, 16 mmol) was added dropwise at RT. The solution was stirred 30 min. at RT. Water (100 mL) was added and the mixture extracted with ethyl acetate (2×150 mL). The combined organic layers were washed with brine, dried with MgSO₄, filtered, and evacuated in vacuo. The residue was purified by silica gel column chromatography (30-50% EA/Hexanes) to provide the title compound as a white powder (720 mg, 67%). ¹H NMR (300 MHz, CDCl₃) δ 3.22 (s, 1H), 3.51 (t, J=5.3 Hz, 1H), 4.80 (d, J=5.2 Hz, 2H), 7.31 (d, J=7.7 Hz, 1H), 7.44 (d, J=7.7 Hz, 1H), 7.71 (t, J=7.8 Hz, 1H).

### Example 74

### 2-[({6-[(4-{3-Chloro-4-[(3-fluorobenzyl)oxy]anilino}-5-pyrimidinyl)ethynyl]-2-pyridinyl}methyl)(methyl)amino]ethanol

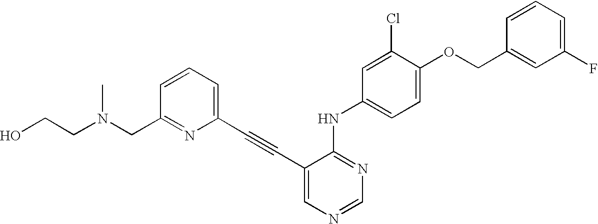

Methanesulfonyl chloride (0.013 mL, 0.2 mmol) was added to a stirring solution of {6-[(4-{3-chloro-[(3-fluorobenzyl)oxy]anilino}-5-pyrimidinyl)ethynyl]-2-pyridinyl}methanol (51 mg, 0.1 mmol) and TEA (0.031 mL, 0.2 mmol) in CH₂Cl₂ (1.5 mL). The mixture was stirred for 15 min. and then 2-(methylamino)ethanol (0.027 mL, 0.3 mmol) was added and the solution was stirred overnight. Water (50 mL) was added and the mixture extracted with CH₂Cl₂ (2×100 mL). The combined organic layers were dried with MgSO₄, filtered, and evacuated in vacuo. The residue was purified by silica gel column chromatography (5% MeOH/CH₂Cl₂) to provide the title compound as a light brown powder (41 mg, 71%). ¹H NMR (300 MHz, CDCl₃) δ 2.63 (s, 3H), 2.89 (m, 2H), 3.37 (t, J=5.2 Hz, 1H), 3.82 (m, 2H), 4.02 (s, 2H), 5.19 (s, 2H), 7.00 (d, J=8.8 Hz, 1H), 7.06 (t, J=8.8 Hz, 1H), 7.26 (m, 2H), 7.39 (m, 2H), 7.52 (d, J=7.7 Hz, 1H), 7.62 (dd, J=8.9, 2.6 Hz, 1H), 7.80 (m, 2H), 8.54 (s, 1H), 8.63 (bs, 1H), 8.66 (s, 1H); ESIMS: 518 (M+H)⁺

### Example 75

### 3-[({6-[(4-{3-Chloro-4-[(3-fluorobenzyl)oxy]anilino}-5-pyrimidinyl)ethynyl]-2-pyridinyl}methyl)amino]propanenitrile

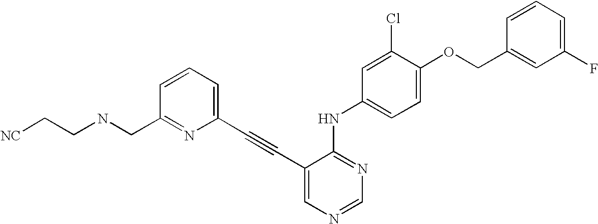

Methanesulfonyl chloride (0.013 mL, 0.2 mmol) was added to a stirring solution of {6-[(4-{3-chloro-4-[(3-fluorobenzyl)oxy]anilino}-5-pyrimidinyl)ethynyl]-2-pyridinyl}methanol (50 mg, 0.1 mmol) and TEA (0.031 mL, 0.2 mmol) in CH₂Cl₂ (1.5 mL). The mixture was stirred for 15 min. and then 3-aminopropanenitrile (0.024 mL, 0.3 mmol) was added and the solution was stirred overnight. Water (50 mL) was added and the mixture extracted with CH₂Cl₂ (2×100 mL). The combined organic layers were dried with MgSO₄, filtered, and evacuated in vacuo. The residue was purified by silica gel column chromatography (5% MeOH/CH₂Cl₂) to provide the title compound as a brown powder (24 mg, 43%). ¹H NMR (400 MHz, DMSO-d₆) δ 2.62 (t, J=6.4 Hz, 2H), 2.76 (t, J=6.4 Hz, 2H), 3.83 (s, 2H), 5.23 (s, 2H), 7.15 (t, J=8.5 Hz, 1H), 7.21 (d, J=8.8 Hz, 1H), 7.29 (m, 2H), 7.41-7.51 (m, 3H), 7.69 (d, J=7.7 Hz, 1H), 7.73 (d, J=2.2 Hz, 1H), 7.85 (t, J=7.8 Hz, 1H), 8.55 (s, 2H), 9.09 (s, 1H); ESIMS: 513 (M+H)⁺

### Example 76

### N-{3-Chloro-4-[(3-fluorobenzyl)oxy]phenyl}-5-{[6-({[2-(4-morpholinyl)ethyl]amino}methyl)-2-pyridinyl]ethynyl}-4-pyrimidinamine

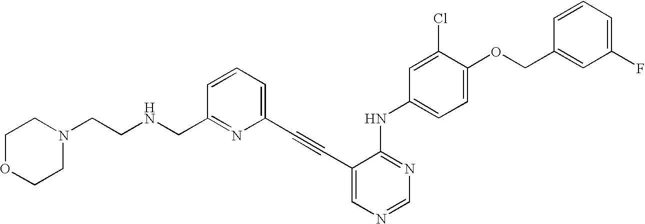

Methanesulfonyl chloride (0.013 mL, 0.2 mmol) was added to a stirring solution of {6-[(4-{3-chloro-4-[(3-fluorobenzyl)oxy]anilino}-5-pyrimidinyl)ethynyl]-2-pyridinyl}methanol (53 mg, 0.1 mmol) and TEA (0.048 mL, 0.3 mmol) in CH₂Cl₂ (1.5 mL). The mixture was stirred for 15 min. and then 2-(4-morpholinyl)ethanamine (0.045 mL, 0.3 mmol) was added and the solution was stirred overnight. Water (50 mL) was added and the mixture extracted with CH₂Cl₂ (2×100 mL). The combined organic layers were dried with MgSO₄, filtered, and evacuated in vacuo. The residue was purified by silica gel column chromatography (5% MeOH/CH₂Cl₂) to provide the title compound as a beige powder (51 mg, 78%). ¹H NMR (300 MHz, DMSO-d₆) δ 2.35 (m, 4H), 2.42 (t, J=6.3 Hz, 2H), 2.64 (t, J=6.3 Hz, 2H), 3.58 (m, 4H), 3.85 (s, 2H), 5.27 (s, 2H), 7.17-7.35 (m, 4H), 7.45-7.57 (m, 3H), 7.72 (d, J=7.7 Hz, 1H), 7.77 (d, J=2.3 Hz, 1H), 7.88 (t, J=7.8 Hz, 1H), 8.59 (s, 1H), 9.13 (s, 1H); ESIMS: 573 (M+H)⁺

### Example 77

### N-{2-[({6-[2-(4-{3-Chloro-4-[(3-fluorobenzyl)oxy]anilino}-5-pyrimidinyl)ethynyl]-2-pyridinyl}methyl)amino]ethyl}acetamide

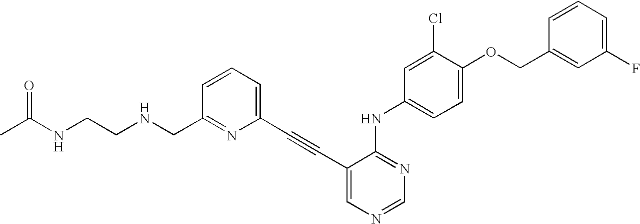

Methanesulfonyl chloride (0.013 mL, 0.2 mmol) was added to a stirring solution of {6-[(4-{3-chloro-4-[(3-fluorobenzyl)oxy]anilino}-5-pyrimidinyl)ethynyl]-2-pyridinyl}methanol (53 mg, 0.1 mmol) and TEA (0.048 mL, 0.3 mmol) in CH₂Cl₂ (1.5 mL). The mixture was stirred for 15 min. and then N-(2-aminoethyl)acetamide (0.035 mL, 0.3 mmol) was added and the solution was stirred overnight. Water (50 mL) was added and the mixture extracted with CH₂Cl₂ (2×100 mL). The combined organic layers were dried with MgSO₄, filtered, and evacuated in vacuo. The residue was purified by silica gel column chromatography (5% MeOH/CH₂Cl₂) to provide the title compound as a beige powder (37 mg, 59%). ¹H NMR (300 MHz, DMSO-d₆) δ 1.81 (s, 3H), 2.60 (t, J=6.5 Hz, 2H), 3.17 (q, J=6.3 Hz, 2H), 3.84 (s, 2H), 5.28 (s, 2H), 7.17-7.35 (m, 4H), 7.45-7.57 (m, 3H), 7.72 (d, J=7.6 Hz, 1H), 7.78 (d, J=2.6 Hz, 1H), 7.86-7.91 (m, 2H), 8.60 (s, 2H), 9.14 (s, 1H); ESIMS: 545 (M+H)⁺

### Example 78

### N-{3-Chloro-4-[(3-fluorobenzyl)oxy]phenyl}-5-{[6-({[3-(1H-imidazol-1-yl)propyl]amino}methyl)-2-pyridinyl]ethynyl}-4-pyrimidinamine

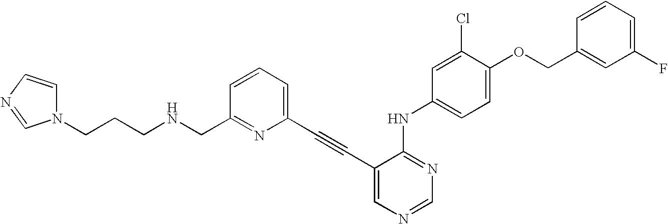

Methanesulfonyl chloride (0.016 mL, 0.2 mmol) was added to a stirring solution of {6-[(4-{3-chloro-4-[(3-fluorobenzyl)oxy]anilino}-5-pyrimidinyl)ethynyl]-2-pyridinyl}methanol (62 mg, 0.1 mmol) and TEA (0.094 mL, 0.7 mmol) in CH₂Cl₂ (2 mL). The mixture was stirred for 15 min. and then 3-(1H-imidazol-1-yl)-1-propanamine (51 mg, 0.4 mmol) was added and the solution was stirred overnight. Water (50 mL) was added and the mixture extracted with CH₂Cl₂ (2×100 mL). The combined organic layers were dried with MgSO₄, filtered, and evacuated in vacuo. The residue was purified by silica gel column chromatography (5% MeOH/CH₂Cl₂) to provide the title compound as a beige powder (47 mg, 61%). ¹H NMR (300 MHz, DMSO-d₆) δ 1.89 (m, 2H), 2.49 (m, 2H), 3.32 (m, 2H), 3.84 (m, 2H), 5.27 (s, 2H), 6.88 (s, 1H), 7.17-7.35 (m, 5H), 7.45-7.56 (m, 3H), 7.63 (s, 1H), 7.72 (d, J=7.5 Hz, 1H), 7.77 (d, J=2.5 Hz, 1H), 7.88 (t, J=7.8 Hz, 1H), 8.59 (s, 2H), 9.18 (s, 1H); ESIMS: 568 (M+H)⁺

### Example 79

### N-{3-chloro-4-[(3-fluorobenzyl)oxy]phenyl}-5-({6-[(methylamino)methyl]-2-pyridinyl}ethynyl)-4-pyrimidinamine

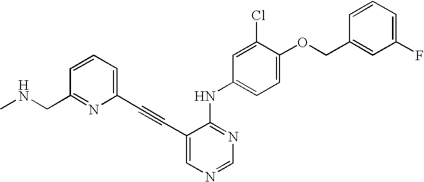

Methanesulfonyl chloride (0.014 mL, 0.2 mmol) was added to a stirring solution of {6-[(4-{3-chloro-4-[(3-fluorobenzyl)oxy]anilino}-5-pyrimidinyl)ethynyl]-2-pyridinyl}methanol (55 mg, 0.1 mmol) and TEA (0.05 mL, 0.4 mmol) in THF (2 mL). The mixture was stirred for 15 min. and methyl amine (2.0 M in THF, 0.6 mL, 1.2 mmol) was added and the solution was stirred overnight. The solvent was evacutated in vacuo and the residue purified by silica gel column chromatography (5% MeOH/CH₂Cl₂) to provide the title compound as a cream powder (37 mg, 65%). ¹H NMR (300 MHz, CDCl₃) δ 2.43 (s, 3H), 3.96 (s, 2H), 5.18 (s, 2H), 6.98 (d, J=8.8 Hz, 1H), 7.06 (dt, J=8.9, 2.3 Hz, 1H), 7.25 (m, 2H), 7.40 (m, 2H), 7.50 (m, 2H), 7.75 (d, J=7.8 Hz, 1H), 7.79 (d, J=2.5 Hz, 1H), 7.89 (s, 1H), 8.56 (s, 1H), 8.68 (s, 1H); APCIMS: 474 (M+H)⁺

### Example 80

### N-{3-Chloro-4-[(3-fluorobenzyl)oxy]phenyl}-5-{[6-(methoxymethyl)-2-pyridinyl]ethynyl}-4-pyrimidinamine

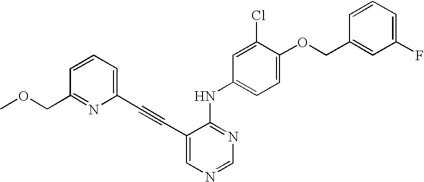

Methanesulfonyl chloride (0.015 mL, 0.2 mmol) was added to a stirring solution of {6-[(4-{3-chloro-4-[(3-fluorobenzyl)oxy]anilino}-5-pyrimidinyl)ethynyl]-2-pyridinyl}methanol (60 mg, 0.1 mmol) and TEA (0.054 mL, 0.4 mmol) in THF (2 mL). The mixture was stirred for 15 min. and sodium methoxide (25% wt. in MeOH, 0.3 mL, 1.3 mmol) was added and the solution was stirred overnight. Water (50 mL) was added and the mixture extracted with ethyl acetate (2×100 mL). The combined organic layers were dried with MgSO₄, filtered, and evacuated in vacuo. The residue was purified by silica gel column chromatography (ethyl acetate) to provide an oil which was triturated (EA/hexanes) and filtered to provide the title compound as a beige powder (45 mg, 73%). ¹H NMR (300 MHz, DMSO-d₆) δ 3.36 (s, 3H), 4.24 (s, 2H), 5.34 (s, 2H), 7.19-7.36 (m, 7H), 7.47-7.59 (m, 3H), 7.87 (t, J=7.8 Hz, 1H), 8.84 (s, 1H), 9.19 (s, 1H); APCIMS: 475 (M+H)⁺

### Example 81

### N-{3-Chloro-4-[(3-fluorobenzyl)oxy]phenyl}-5-{[2-(methylsulfanyl)-4-pyrimidinyl]ethynyl}-4-pyrimidinamine

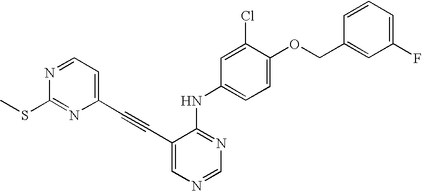

a) In a similar manner as described in Example 24c, from 4-ethynyl-2-(methylsulfanyl)pyrimidine (92 mg, 0.6 mmol) was obtained the title compound as a beige solid (141 mg, 58%). ¹H NMR (300 MHz, DMSO-d₆) δ 2.57 (s, 3H), 5.28 (s, 2H), 7.18-7.35 (m, 3H), 7.45-7.54 (m, 2H), 7.60 (d, J=5.1 Hz, 1H), 7.75 (d, J=2.4 Hz, 1H), 8.65 (d, J=9.0 Hz, 2H), 8.76 (d, J=5.1 Hz, 1H), 9.26 (s, 1H); ESIMS: 476 (M−H)⁻

b) 4-Ethynyl-2-(methylsulfanyl)pyrimidine 4-iodo-2-(methylsulfanyl)pyrimidine (16.6 g, 66 mmol), TMS acetylene (19 mL, 135 mmol), Pd(PPh₃)₂Cl₂ (2.3 g, 3.3 mmol), CuI (1.25 g, 6.6 mmol), and TEA (28 mL, 202 mmol) were added to DMF (150 mL) at RT. The mixture was heated at 50° C. for 1 h. The mixture was cooled and then water (300 mL) was added and the mixture extracted with ethyl acetate (2×400 mL). The combined organic layers were washed with brine, dried with MgSO₄, filtered, and evacuated in vacuo. The residue was passed through a pad of silica gel (10% EA/Hexanes) to provide the silyl protected intermediate as a dark brown oil. The oil was dissolved in MeOH (60 mL) and KF (3.83 g, 66 mmol) was added dropwise at RT. The solution was stirred 30 min. at RT. Water (100 mL) was added and the mixture extracted with ethyl acetate (2×400 mL). The combined organic layers were washed with brine, dried with MgSO₄, filtered, and evacuated in vacuo. The residue was purified by silica gel column chromatography (20-40% EA/Hexanes) to provide a yellow solid. The solid was triturated (Et₂O/hexanes) and filtered to provide the 5.2 g (53%) of the product as a white powder.

### Example 82

### N-{3-Chloro-4-[(3-fluorobenzyl)oxy]phenyl}-5-({6-[(dimethylamino)methyl]-2-pyridinyl}ethynyl)₄-pyrimidinamine

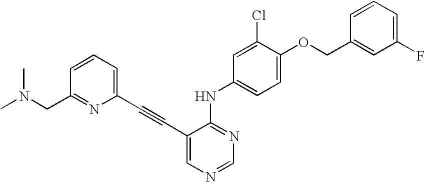

Methanesulfonyl chloride (0.017 mL, 0.2 mmol) was added to a stirring solution of {6-[(4-{3-chloro-4-[(3-fluorobenzyl)oxy]anilino}-5-pyrimidinyl)ethynyl]-2-pyridinyl}methanol (69 mg, 0.1 mmol) and TEA (0.042 mL, 0.3 mmol) in CH₂Cl₂ (2 mL). The mixture was stirred for 15 min. and dimethyl amine (2.0M in MeOH, 0.75 mL, 1.5 mmol) was added and the solution was stirred overnight. Water (50 mL) was added and the mixture extracted with ethyl acetate (2×100 mL). The combined organic layers were dried with MgSO₄, filtered, and evacuated in vacuo. The residue was purified by silica gel column chromatography (5% MeOH/CH₂Cl₂) to provide the title compound as a cream powder (54 mg, 74%). ¹H NMR (400 MHz, DMSO-d₆) δ 2.18 (s, 6H), 3.52 (s, 2H), 5.23 (s, 2H), 7.15 (dt, J=8.3, 2.0 Hz, 1H), 7.21 (d, J=9.0 Hz, 1H), 7.28 (m, 2H), 7.44 (m, 2H), 7.51 (dd, J=8.9, 2.3 Hz, 1H), 7.69 (d, J=7.6 Hz, 1H), 7.73 (d, J=2.2 Hz, 1H), 7.84 (t, J=7.7 Hz, 1H), 8.56 (s, 2H), 9.10 (s, 1H); ESIMS: 488 (M+H)⁺

### Example 83

### N-Benzyl-N-({6-[(4-{3-chloro-4-[(3-fluorobenzyl)oxy]anilino}-5-pyrimidinyl)ethynyl]-2-pyridinyl}methyl)amine

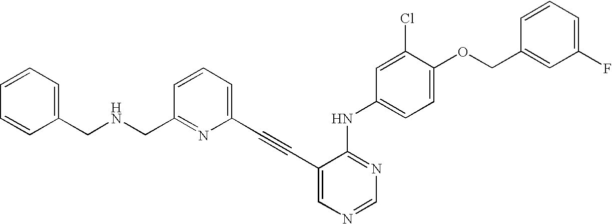

Methanesulfonyl chloride (0.017 mL, 0.2 mmol) was added to a stirring solution of {6-[(4-{3-chloro-4-[(3-fluorobenzyl)oxy]anilino}-5-pyrimidinyl)ethynyl]-2-pyridinyl}methanol (69 mg, 0.1 mmol) and TEA (0.042 mL, 0.3 mmol) in CH₂Cl₂ (2 mL). The mixture was stirred for 15 min. and benzyl amine (0.164 mL, 1.5 mmol) was added and the solution was stirred overnight. Water (50 mL) was added and the mixture extracted with EtOAc (2×100 mL). The combined organic layers were dried with MgSO₄, filtered, and evacuated in vacuo. The residue was purified by silica gel column chromatography (5% MeOH/CH₂Cl₂) to provide the title compound as a brown powder (40 mg, 49%). ¹H NMR (300 MHz, DMSO-d₆) δ 3.75 (s, 2H), 3.84 (s, 2H), 5.27 (s, 2H), 7.17-7.39 (m, 9H), 7.45-7.57 (m, 3H), 7.72 (d, J=7.7 Hz, 1H), 7.78 (d, J=2.4 Hz, 1H), 7.89 (t, J=7.9 Hz, 1H), 8.59 (s, 2H), 9.14 (s, 1H); ESIMS: 550 (M+H)⁺

### Example 84

### N-{3-Chloro-4-[(3-fluorobenzyl)oxy]phenyl}-5-[(6-{[(2-methoxyethyl)amino]methyl}-2-pyridinyl)ethynyl]-4-pyrimidinamine

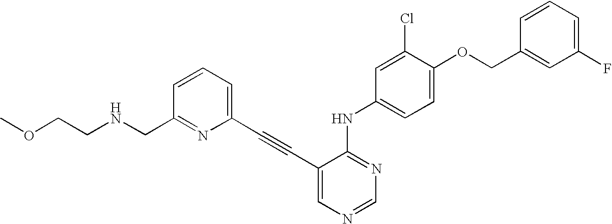

Methanesulfonyl chloride (0.017 mL, 0.2 mmol) was added to a stirring solution of {6-[(4-{3-chloro-4-[(3-fluorobenzyl)oxy]anilino}-5-pyrimidinyl)ethynyl]-2-pyridinyl}methanol (69 mg, 0.1 mmol) and TEA (0.042 mL, 0.3 mmol) in CH₂Cl₂ (2 mL). The mixture was stirred for 15 min. and 2-methoxyethanamine (0.13 mL, 1.5 mmol) was added and the solution was stirred overnight. Water (50 mL) was added and the mixture extracted with EtOAc (2×100 mL). The combined organic layers were dried with MgSO₄, filtered, and evacuated in vacuo. The residue was purified by silica gel column chromatography (2% MeOH/CH₂Cl₂) to provide the title compound as a beige powder (45 mg, 58%). ¹H NMR (400 MHz, DMSO-d₆) δ 2.66 (t, J=5.6 Hz, 2H), 3.22 (s, 3H), 3.39 (t, J=5.6 Hz, 2H), 3.81 (s, 2H), 5.23 (s, 2H), 7.16 (dt, J=8.7, 2.4 Hz, 1H), 7.21 (d, J=9.0 Hz, 1H), 7.26-7.30 (m, 2H), 7.41-7.47 (m, 2H), 7.51 (dd, J=9.0, 2.6 Hz, 1H), 7.67 (d, J=7.5 Hz, 1H), 7.73 (d, J-2.5 Hz, 1H), 7.83 (t, J=7.7 Hz, 1H), 8.55 (s, 2H), 9.09 (s, 1H); ESIMS: 518 (M+H)⁺

### Example 85

### 5-{[6-(Aminomethyl)-2-pyridinyl]ethynyl}-N-{3-chloro-4-[(3-fluorobenzyl) oxy]phenyl}-4-pyrimidinamine*2TFA

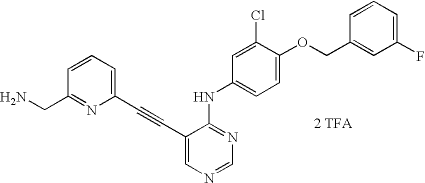

a) TFA (0.25 mL) was added to a solution of di(tert-butyl) {6-[(4-{3-chloro-4-[(3-fluorobenzyl)oxy]anilino}-5-pyrimidinyl)ethynyl]-2-pyridinyl}methylimido dicarbonate (63 mg, 0.1 mmol) and was stirred for 1 h at RT. The solvents were evacuated in vacuo and the thick oil was coevaporated x3 with CH₂Cl₂. The resulting solid was placed under hi-vacuum overnight and then recovered to provide the title compound as a yellow powder (67 mg, 97%). ¹H NMR (300 MHz, DMSO-d₆) δ 4.27 (s, 2H), 5.28 (s, 2H), 7.17-7.35 (m, 4H), 7.45-7.56 (m, 3H), 8.38 (bs, 3H), 8.55 (s, 1H), 8.62 (s, 1H), 9.16 (s, 1H); ESIMS: 460 (M+H)⁺

b) Di(tert-butyl)-{6-[(4-{3-chloro-4-[(3-fluorobenzyl)oxy]anilino}-5-pyrimidinyl)ethynyl]-2-pyridinyl}methylimidodicarbonate Methanesulfonyl chloride (0.023 mL, 0.3 mmol) was added to a stirring solution of {6-[(4-{3-chloro-4-[(3-fluorobenzyl)oxy]anilino}-5-pyrimidinyl)ethynyl]-2-pyridinyl}methanol (92 mg, 0.2 mmol) and TEA (0.055 mL, 0.4 mmol) in DMF (1 mL). In a separate flask NaH (60% dispersion, 40 mg, 1.0 mmol) was added to a solution of di(tert-butyl) imidodicarbonate (217 mg, 1.0 mmol) in DMF (1 mL). Each reaction was stirred for 15 min. at RT and then the mesylate intermediate was syringed dropwise into the generated anion dropwise. The mixture was stirred at RT overnight. Water (50 mL) was added and the mixture was extracted with EtOAc (2×100 mL). The combined organic layers were dried with MgSO₄, filtered, and evacuated in vacuo. The residue was purified by silica gel column chromatography (50% EtOAc/hexanes-100% EtOAc) to provide the title compound as a yellow oil (63 mg, 48%); ESIMS: 542 (M+H)⁺

### Example 86

### N-({6-[(4-{3-Chloro-4-[(3-fluorobenzyl)oxy]anilino}-5-pyrimidinyl)ethynyl]-2-pyridinyl}methyl)-N′-(2-cyanoethyl)urea

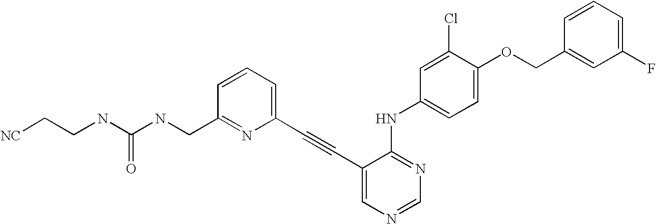

1,1′-Carbonyldiimidazole (11 mg, 0.1 mmol) was added to 5-{[6-(aminomethyl)-2-pyridinyl]ethynyl}N-{3-chloro-4-[(3-fluorobenzyl)oxy]phenyl}-4-pyrimidinamine bis TFA (40 mg, 0.1 mmol) and DIEA (0.05 mL, 0.3 mmol) in CHCl₃ at RT. After stirring for 15 min. at RT, 3-aminopropanenitrile (0.013 mL, 0.2 mmol) was added. The solution was stirred overnight at RT. Water (50 mL) was added and the mixture was extracted with CH₂Cl₂ (150 mL). The combined organic layers were dried with MgSO₄, filtered, and evacuated in vacuo. The residue was purified by silica gel column chromatography (EtOAc) and then triturated (Et₂O) and filtered to provide the title compound as a brown powder (10 mg, 30%). ¹H NMR (400 MHz, DMSO-d₆) δ 2.60 (t, J=6.4 Hz, 2H), 3.26 (q, J=6.3 Hz, 2H), 4.31 (d, J=5.9 Hz, 2H), 5.23 (s, 2H), 6.51 (t, J=6.0 Hz, 1H), 6.73 (t, J=6.0 Hz, 1H), 7.16 (dt, J=8.7, 2.6 Hz, 1H), 7.21 (d, J=9.0 Hz, 1H), 7.29 (m, 3H), 7.45 (m, 1H), 7.50 (dd, J=9.0, 2.6 Hz, 1H), 7.68 (d, J=7.7 Hz, 1H), 7.73 (d, J=2.5 Hz, 1H), 7.83 (t, J=7.8 Hz, 1H), 8.55 (s, 2H), 9.11 (s, 1H); ESIMS: 556 (M+H)⁺

### Example 87

### N′-({6-[(4-{3-Chloro-4-[(3-fluorobenzyl)oxy]anilino}-5-pyrimidinyl)ethynyl]-2-pyridinyl}methyl)-N-(2-hydroxyethyl)-N-methylurea

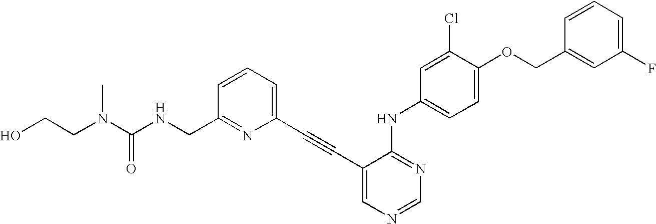

1,1′-Carbonyldiimidazole (12 mg, 0.1 mmol) was added to 5-{[6-(aminomethyl)-2-pyridinyl]ethynyl}-N-{3-chloro-4-[(3-fluorobenzyl)oxy]phenyl}-4-pyrimidinamine bis TFA (42 mg, 0.1 mmol) and DIEA (0.053 mL, 0.3 mmol) in CHCl₃ at RT. After stirring for 15 min. at RT, 2-(methylamino)ethanol (0.015 mL, 0.2 mmol) was added. The solution was stirred overnight at RT. Water (50 mL) was added and the mixture was extracted with CH₂Cl₂ (150 mL). The combined organic layers were dried with MgSO₄, filtered, and evacuated in vacuo. The residue was purified by silica gel column chromatography (5% MeOH/CH₂Cl₂) to provide the title compound as a white solid (13 mg, 38%). ¹H NMR (400 MHz, DMSO-d₆) δ 2.87 (s, 3H), 3.27 (m, 2H), 3.48 (q, J=5.7 Hz, 2H), 4.30 (d, J=5.7 Hz, 2H), 4.71 (t, J=5.2 Hz, 1H), 5.23 (s, 2H), 6.96 (t, J=6.0 Hz, 1H), 7.16 (t, J=8.6 Hz, 1H), 7.21 (d, J=9.0 Hz, 1H), 7.29 (m, 3H), 7.45 (m, 1H), 7.50 (dd, J=8.9, 2.5 Hz, 1H), 7.66 (d, J=7.5 Hz, 1H), 7.73 (d, J=2.5 Hz, 1H), 7.82 (t, J=7.8 Hz, 1H), 8.56 (s, 2H), 9.10 (s, 1H); ESIMS: 561 (M+H)⁺

### Example 88

### N-({6-[(4-{3-Chloro-4-[(3-fluorobenzyl)oxy]anilino}-5-pyrimidinyl)ethynyl]-2-pyridinyl}methyl)-N′-[2-(methylsulfonyl)ethyl]urea

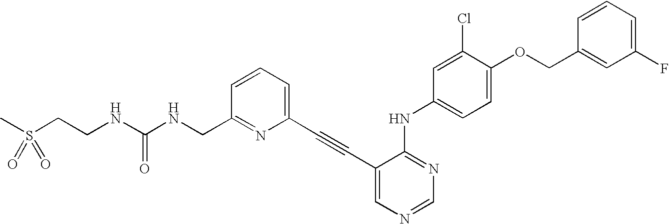

1,1′-Carbonyldiimidazole (13 mg, 0.1 mmol) was added to 5-{[6-(aminomethyl)-2-pyridinyl]ethynyl}N-{3-chloro-4-[(3-fluorobenzyl)oxy]phenyl}-4-pyrimidinamine bis TFA (47 mg, 0.1 mmol) and DIEA (0.06 mL, 0.3 mmol) in CHCl₃ (1 mL) at RT. After stirring for 15 min. at RT, 2-(methylsulfonyl)ethanamine (25 mg, 0.2 mmol) was added. The solution was stirred overnight at RT. EtOAc (2 mL) was added to the mixture and the flask was sonicated until a thick white precipitate formed. The solid was filtered and rinsed with EtOAc and then hexanes. Recovery of the material provided the title compound as a white solid (17 mg, 40%). ¹H NMR (400 MHz, DMSO-d₆) δ 2.97 (s, 3H), 3.21 (t, J=6.6 Hz, 2H), 3.44 (q, J=6.3 Hz, 2H), 4.30 (d, J=6.0 Hz, 2H), 5.23 (s, 2H), 6.35 (t, J=6.0 Hz, 1H), 6.79 (t, J=6.0 Hz, 1H), 7.16 (dt, J=9.2, 2.4 Hz, 1H), 7.21 (d, J=9.1 Hz, 1H), 7.29 (m, 3H), 7.45 (m, 1H), 7.50 (dd, J=9.0, 2.6 Hz, 1H), 7.68 (d, J=7.7 Hz, 1H), 7.73 (d, J=2.6 Hz, 1H), 7.83 (t, J=7.8 Hz, 1H), 8.55 (s, 2H), 9.10 (s, 1H); ESIMS: 609 (M+H)⁺

### Example 89

### N-({6-[(4-{3-Chloro-4-[(3-fluorobenzyl)oxy]anilino}-5-pyrimidinyl)ethynyl]-2-pyridinyl}methyl)-N′-[2-(4-morpholinyl)ethyl]urea

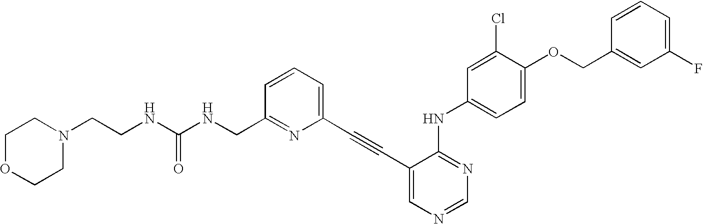

1,1′-Carbonyldiimidazole (13 mg, 0.1 mmol) was added to 5-{[6-(aminomethyl)-2-pyridinyl]ethynyl}-N-{3-chloro-4-[(3-fluorobenzyl)oxy]phenyl}-4-pyrimidinamine bis TFA (45 mg, 0.1 mmol) and DIEA (0.057 mL, 0.3 mmol) in CHCl₃ at RT. After stirring for 15 min. at RT, 2-(4-morpholinyl)ethanamine (0.05 mL 0.2 mmol) was added. The solution was stirred overnight at RT. Water (50 mL) was added and the mixture was extracted with CH₂Cl₂ (150 mL). The combined organic layers were dried with MgSO₄, filtered, and evacuated in vacuo. The residue was purified by silica gel column chromatography (0-10% MeOH/CH₂Cl₂) to provide a light yellow oil. The residue was lyophylyzed (MeOH/H₂O) to give the title compound as a white solid (24 mg, 60%). ¹H NMR (400 MHz, DMSO-d₆) δ 2.36 (m, 6H), 3.13 (q, J=6.1 Hz, 2H), 3.55 (t, J=4.5 Hz, 1H), 4.29 (d, J=6.0 Hz, 2H), 5.23 (s, 2H), 6.05 (t, J=5.4 Hz, 1H), 6.66 (t, J=5.4 Hz, 1H), 7.16 (dt, J=9.2, 2.6 Hz, 1H), 7.21 (d, J=9.0 Hz, 1H), 7.29 (m, 3H), 7.45 (m, 1H), 7.50 (dd, J=8.9, 2.5 Hz, 1H), 7.68 (d, J=7.5 Hz, 1H), 7.73 (d, J=2.5 Hz, 1H), 7.83 (t, J=7.8 Hz, 1H), 8.55 (s, 2H), 9.10 (s, 1H); ESIMS: 616 (M+H)⁺

### Example 90

### N-({6-[(4-{3-Chloro-4-[(3-fluorobenzyl)oxy]anilino}-5-pyrimidinyl)ethynyl]-2-pyridinyl}methyl)-N′-methylurea

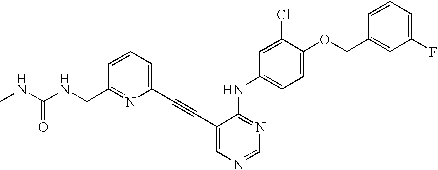

1,1′-Carbonyldiimidazole (14 mg, 0.1 mmol) was added to 5-{[6-(aminomethyl)-2-pyridinyl]ethynyl}-N-{3-chloro-4-[(3-fluorobenzyl)oxy]phenyl}-4-pyrimidinamine bis TFA (50 mg, 0.1 mmol) and DIEA (0.063 mL, 0.4 mmol) in CHCl₃ at RT. After stirring for 15 min. at RT, methylamine (8 M in ethanol, 0.030 mL, 0.2 mmol) was added. The solution was stirred overnight at RT. Et₂O (3 mL) was added to the flask and the mixture was sonicated. The resulting precipitate was filtered and rinsed with Et₂O to give the title compound as an off-white solid (35 mg, 92%). ¹H NMR (300 MHz, DMSO-d₆) δ 2.60 (d, J=4.4 Hz, 3H), 4.33 (d, J=5.9 Hz, 2H), 5.28 (s, 2H), 6.04 (q, J=4.6 Hz, 1H), 6.59 (t, J=5.9 Hz, 1H), 7.18-7.35 (m, 5H), 7.45-7.57 (m, 2H), 7.72 (d, J=7.6 Hz, 1H), 7.77 (d, J=2.4 Hz, 1H), 7.87 (t, J=7.9 Hz, 1H), 8.60 (s, 2H), 9.15 (s, 1H); ESIMS: 517 (M+H)⁺

### Example 91

### N-({6-[(4-{3-Chloro-4-[(3-fluorobenzyl)oxy]anilino}-5-pyrimidinyl)ethynyl]-2-pyridinyl}methyl)-N′-(2-methoxyethyl)urea

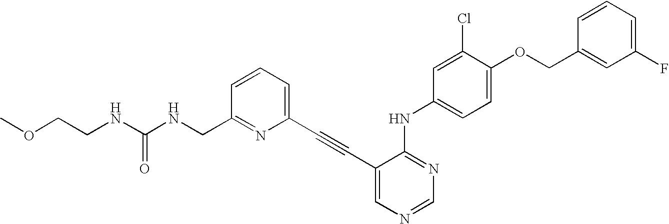

1,1′-Carbonyldiimidazole (13 mg, 0.1 mmol) was added to 5-{[6-(aminomethyl)-2-pyridinyl]ethynyl}-N-{3-chloro-4-[(3-fluorobenzyl)oxy]phenyl)-4-pyrimidinamine bis TFA (45 mg, 0.1 mmol) and DIEA (0.057 mL, 0.3 mmol) in CHCl₃ at RT. After stirring for 15 min. at RT, 2-methoxyethylamine (0.013 mL, 0.2 mmol) was added. The solution was stirred overnight at RT. Et₂O (3 mL) was added to the flask and the mixture was sonicated. The resulting precipitate was filtered and rinsed with Et₂O to give the title compound as an off-white solid (33 mg, 90%). ¹H NMR (400 MHz, DMSO-d₆) δ 3.16 (q, J=5.6 Hz, 2H), 3.22 (s, 3H), 3.29 (m, 2H), 4.29 (d, J=5.9 Hz, 2H), 5.23 (s, 2H), 7.16 (dt, J=8.6 Hz, 1H), 7.21 (d, J=9.0 Hz, 1H), 7.29 (m, 3H), 7.44 (m, 1H), 7.50 (dd, J=8.9, 2.5 Hz, 1H), 7.67 (d, J=7.5 Hz, 1H), 7.73 (d, J=2.4 Hz, 1H), 7.83 (t, J=7.8 Hz, 1H), 8.55 (s, 2H), 9.10 (s, 1H); ESIMS: 561 (M+H)⁺

### Example 92

### N-{3-Chloro-4-[(3-fluorobenzyl)oxy]phenyl}-5-([6-(1-piperidinylmethyl)-2-pyridinyl]ethynyl}-4-pyrimidinamine

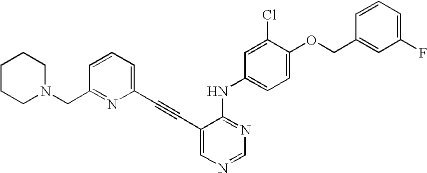

Methanesulfonyl chloride (0.025 mL, 0.3 mmol) was added to a stirring solution of {6-[(4-{3-chloro-4-[(3-fluorobenzyl)oxy]anilino-5-pyrimidinyl)ethynyl]-2-pyridinyl}methanol (100 mg, 0.2 mmol) and TEA (0.060 mL, 0.4 mmol) in CH₂Cl₂ (2 mL). The mixture was stirred for 15 min. and piperidine (0.110 mL, 1.1 mmol) was added and the solution was stirred overnight. Water (50 mL) was added and the mixture extracted with CH₂Cl₂ (2×100 mL). The combined organic layers were dried with MgSO₄, filtered, and evacuated in vacuo. The residue was purified by silica gel column chromatography (2% MeOH/CH₂Cl₂) to provide the title compound as a beige powder (47 mg, 41%). ¹H NMR (400 MHz, DMSO-d₆) δ 1.38 (m, 2H), 1.49 (m, 4H), 2.35 (bs, 4H), 3.53 (bs, 2H), 5.23 (s, 2H), 7.15 (dt, J=8.6, 2.3 Hz, 1H), 7.21 (d, J=9.0 Hz, 1H), 7.29 (m, 2H), 7.45 (m, 2H), 7.50 (dd, J=8.9, 2.5 Hz, 1H), 7.68 (d, J=7.5 Hz, 1H), 7.73 (d, J=2.6 Hz, 1H), 7.83 (t, J=7.8 Hz, 1H), 8.56 (s, 2H), 9.09 (s, 1H); APCIMS: 528 (M+H)⁺

### Example 93

### N-(3-Chloro-4-[(3-fluorobenzyl)oxy]phenyl}-5-({6-[(4-methyl-1-piperazinyl)methyl]-2-pyridinyl}ethynyl)-4-pyrimidinamine

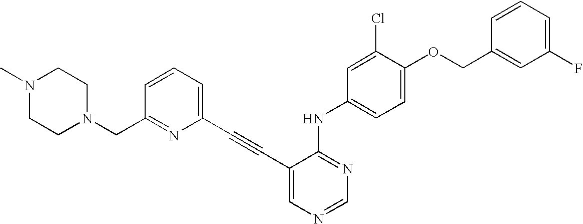

Methanesulfonyl chloride (0.025 mL, 0.3 mmol) was added to a stirring solution of {6-[(4-{3-chloro-4-[(3-fluorobenzyl)oxy]anilino}-5-pyrimidinyl)ethynyl]-2-pyridinyl}methanol (100 mg, 0.2 mmol) and TEA (0.060 mL, 0.4 mmol) in CH₂Cl₂ (2 mL). The mixture was stirred for 15 min. and 1-methylpiperazine (0.121 mL, 1.1 mmol) was added and the solution was stirred overnight. Water (50 mL) was added and the mixture extracted with CH₂Cl₂ (2×100 mL). The combined organic layers were dried with MgSO₄, filtered, and evacuated in vacuo. The residue was purified by silica gel column chromatography (5% MeOH/CH₂Cl₂) to provide the title compound as a beige powder (46 mg, 39%). ¹H NMR (400 MHz, DMSO-d₈) δ 2.24 (s, 3H), 2.48 (m, 8H), 3.59 (s, 2H), 5.23 (s, 2H), 7.16 (t, J=8.6 Hz, 1H), 7.21 (d, J=9.0 Hz, 1H), 7.29 (m, 2H), 7.44 (m, 2H), 7.50 (dd, J=9.0, 2.1 Hz, 1H), 7.70 (d, J=7.7 Hz, 1H), 7.72 (d, J=2.2 Hz, 1H), 7.85 (t, J=7.8 Hz, 1H), 8.56 (s, 2H), 9.09 (s, 1H). APCIMS: 543 (M+H)⁺

### Example 94

### N-{3-Chloro-4-[(3-fluorobenzyl)oxy]phenyl}-5-{([6-(4-morpholinyl-methyl)-2-pyridinyl]ethynyl}-4-pyrimidinamine

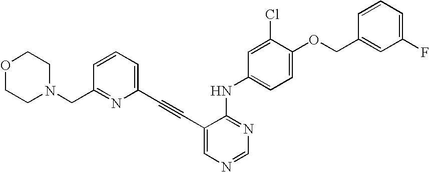

Methanesulfonyl chloride (0.025 mL, 0.3 mmol) was added to a stirring solution of {6-[(4-{3-chloro-4-[(3-fluorobenzyl)oxy]anilino}-5-pyrimidinyl)ethynyl]-2-pyridinyl}methanol (100 mg, 0.2 mmol) and TEA (0.060 mL, 0.4 mmol) in CH₂Cl₂ (2 mL). The mixture was stirred for 15 min. and morpholine (0.095 mL, 1.1 mmol) was added and the solution was stirred overnight. Water (50 mL) was added and the mixture extracted with CH₂Cl₂ (2×100 mL). The combined organic layers were dried with MgSO₄, filtered, and evacuated in vacuo. The residue was purified by silica gel column chromatography (5% MeOH/CH₂Cl₂) to provide the title compound as a beige powder (52 mg, 45%). ¹H NMR (300 MHz, DMSO-d₆) δ2.44 (bs, 4H), 3.63 (bm, 6H), 5.27 (s, 2H), 7.17-7.35 (m, 4H), 7.45-7.56 (m, 3H), 7.76 (m, 2H), 8.61 (bs, 2H), 9.14 (s, 1H); APCIMS: 530 (M+H)⁺

### Example 95

### N-{3-Chloro-4-[(3-fluorobenzyl)oxy]phenyl}-5-{[6-(1-pyrrolidinyl-methyl)-2-pyridinyl]ethynyl}-4-pyrimidinamine

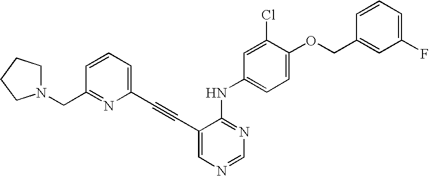

Methanesulfonyl chloride (0.025 mL, 0.3 mmol) was added to a stirring solution of {6-[(4-{3-chloro-4-[(3-fluorobenzyl)oxy]anilino}-5-pyrimidinyl)ethynyl]-2-pyridinyl}methanol (100 mg, 0.2 mmol) and TEA (0.060 mL, 0.4 mmol) in CH₂Cl₂ (2 mL). The mixture was stirred for 15 min. and pyrrolidine (0.090 mL, 1.1 mmol) was added and the solution was stirred overnight. Water (50 mL) was added and the mixture extracted with CH₂Cl₂ (2×100 mL). The combined organic layers were dried with MgSO₄, filtered, and evacuated in vacuo. The residue was purified by silica gel column chromatography (2% MeOH/CH₂Cl₂) to provide the title compound as a beige powder (59 mg, 53%). ¹H NMR (400 MHz, DMSO-d₆) δ 1.73 (bs, 4H), 2.58 (bs, 4H), 3.82 (bs, 2H), 5.23 (s, 2H), 7.16 (dt, J=8.6, 2.5 Hz, 1H), 7.21 (d, J=9.0 Hz, 1H), 7.29 (m, 2H), 7.46 (m, 2H), 7.50 (dd, J=9.0, 2.6 Hz, 1H), 7.72 (m, 2H), 7.86 (t, J=7.8 Hz, 1H), 8.55 (s, 2H), 9.10 (s, 1H); APCIMS: 514 (M+H)⁺

### Example 96

### N-{3-Chloro-4-[(3-fluorobenzyl)oxy]phenyl}-5-{[6-(1-piperazinylmethyl)-2-pyridinyl]ethynyl}-4-pyrimidinamine

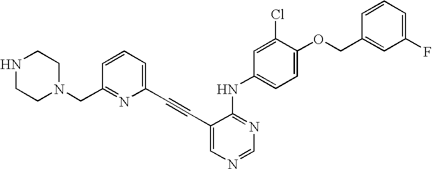

Methanesulfonyl chloride (0.025 mL, 0.3 mmol) was added to a stirring solution of {6-[(4-{3-chloro-4-[(3-fluorobenzyl)oxy]anilino}-5-pyrimidinyl)ethynyl]-2-pyridinyl}methanol (100 mg, 0.2 mmol) and TEA (0.060 mL, 0.4 mmol) in CH₂Cl₂ (2 mL). The mixture was stirred for 15 min. and pyrrolidine (93 mg, 1.1 mmol) was added and the solution was stirred overnight. Water (50 mL) was added and the mixture extracted with CH₂Cl₂ (2×100 mL). The combined organic layers were dried with MgSO₄, filtered, and evacuated in vacuo. The residue was purified by silica gel column chromatography (10% MeOH/CH₂Cl₂) to provide the title compound as a beige powder (41 mg, 36%). APCIMS: 529 (M+H)⁺

### Example 97

### 4-Amino-2-{[4-({3-chloro-4-[(4-fluorobenzyl)oxy]phenyl}amino)pyrimidin-5-yl]ethynyl}pyrimidine-5-carbonitrile

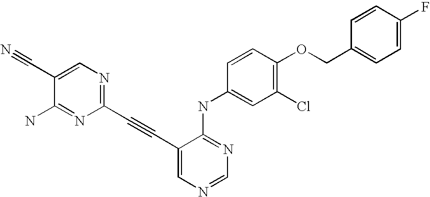

a) In a similar manner as described in Example 24c, from 4-amino-2-ethynyl-5-pyrimidinecarbonitrile (50 mg, 0.3 mmol) was obtained the title compound as a beige solid (20 mg, 12%). ¹H NMR (300 MHz, DMSO-d₆) δ 5.23 (s, 2H), 7.22-7.13 (m, 2H), 7.31-7.27 (m, 2H), 7.52-7.42 (m, 2H), 7.75-7.74 (d, J=2.48 Hz, 1H), 8.60 (bs, 2H or 1 δ δ δ), 8.68 (s, 1H), 9.23 (s, 1H); AP MS: 472 (M+H)⁺

b) 4-Amino-2-ethynyl-5-pyrimidinecarbonitrile A mixture of THF (25 mL) and MeOH (2 mL) was added to crude 4-amino-2-[(trimethylsilyl)ethynyl]-5-pyrimidinecarbonitrile product (850 mg, 3.94 mmol). KF (1.14 g, 19 mmol) was added and the reaction stirred for 2 hours at RT. The solvent was removed in vacuo and the oil passed through a pad of silica gel with ethyl acetate as the eluent. This material was dried on a vacuum pump (RT, 1 torr) overnight providing 0.65 g (>95%).

c) 4-Amino-2-[(trimethylsilyl)ethynyl]-5-pyrimidinecarbonitrile 4-amino-2-bromopyrimidine-5-carbonitrile (1 g, 5.0 mmol), TMS-acetylene (1.4 mL, 10 mmol), Pd(PPh₃)₂Cl₂ (175 mg, 0.25 mmol), CuI (95 mg, 0.5 mmol) and TEA (2.1 mL, 15.0 mmol) in 25 mL THF was heated at 55° C. for 1 hour. The crude material (1.06 g (>95%), 4-amino-2-[(trimethylsilyl)ethynyl]pyrimidine-5-carbonitrile was used in reaction a).

### Example 98

### 2-{[4-({3-Chloro-4-[(3-fluorobenzyl)oxy]phenyl}amino)pyrimidin-5-yl]ethynyl}-4-{[2-(methylsulfonyl)ethyl]amino}pyrimidine-5-carbonitrile

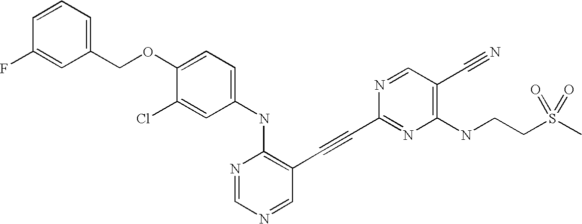

4-Amino-2-{[4-({3-chloro-4-[(4-fluorobenzyl)oxy]phenyl}amino)pyrimidin-5-yl]ethynyl}pyrimidine-5-carbonitrile (20 mg, 0.0425 mmol), NaH (4.5 mg, 0.106 mmol, 60% w/w) and methyl vinyl sulfone (0.011 mL, 0.127 mmol) were combined in DMF (0.5 mL) at RT. The title compound was obtained as a brown solid (8 mg, 33%). ¹H NMR (300 MHz, CDCl₃) δ 2.92-3.08 (m, 3H), 3.44-3.48 (m, 2H), 4.24-4.26 (m, 2H), 5.20 (s, 2 h), 6.7-6.6 (m, 1H), 6.99-7.02 (m, 1H), 7.30-7.23 (m, 1H), 7.42-7.37 (m, 1H), 7.52-7.45 (m, 1H), 7.78-7.77 (d, J=2.3, 1H), 7.96 (m, 1H), 8.73-8.53 (m, 3H); APCl+MS: 578 (M+H)⁺

### Example 99

### 4-{[4-({3-Chloro-4-[(3-fluorobenzyl)oxy]phenyl}amino)pyrimidin-5-yl]ethynyl}pyrimidin-2-amine

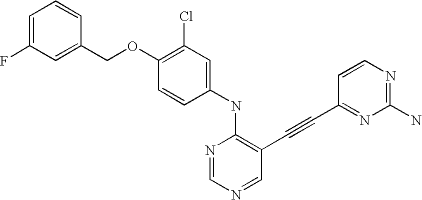

a) In a similar manner as described in Example 24c, from 4-ethynyl-2-pyrimidinamine (180 mg, 1.5 mmol) was obtained the title compound as a brown solid (195 mg, 38%). ¹H NMR (300 MHz, DMSO-d₆) δ 5.28 (s, 2H), 6.88-6.86 (m, 2 h), 6.99-6.9 (m, 1H), 7.35-7.18 (m, 3H), 7.55-7.45 (m, 2H), 7.76 (d, J=2.5 Hz, 1H), 8.36-8.34 (m, 1H), 8.62 (bs, 2H), 9.18 (s, 1H); APCl+MS: 447 (M+H)⁺

b) 4-Ethynyl-2-pyrimidinamine To a solution of 4-iodopyrimidin-2-amine (400 mg, 1.8 mmol) in THF (10 mL) was added Pd(PPh₃)₂Cl₂ (63 mg, 0.09 mmol), CuI (34 mg, 0.18 mmol), TMS-acetylene (510 μL, 3.6 mmol), and TEA (750 μL, 5.4 mmol). The mixture was heated to 50° C. for 1 hr. The solvent was removed in vacuo. THF (8 mL) and MeOH (1 mL) was added to the crude material followed by KF (313 mg, 5.4 mmol). The material was concentrated and used crude in subsequent reactions.

### Example 100

### N-(6-{[4-({3-Chloro-4-[(3-fluorobenzyl)oxy]phenyl}amino)pyrimidin-5-yl]ethynyl}pyridin-2-yl)-2,2,2-trifluoroacetamide

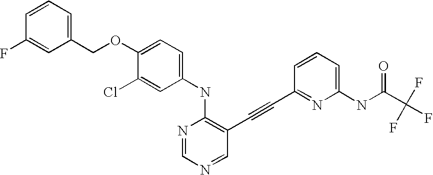

a) In a similar manner as described in Example 24c, from N-(6-ethynyl-2-pyridinyl)-2,2,2-trifluoroacetamide (˜1.0 mmol, crude from part b below) was obtained the title compound as a beige solid (120 mg, 30%). ¹H NMR (300 MHz, DMSO-d₆) δ 5.28 (s, 2H), 7.33 (m, 4H), 7.50 (m, 2H), 7.70 (m, 2H), 8.04 (d, J=4.1 Hz, 2H), 8.58 (bs, 1H), 8.61 (bs, 1H), 9.17 (s, 1H), 12.34 (s, 1H); ESIMS: 542 (M+H)⁺

b) N-(6-Ethynyl-2-pyridinyl)-2,2,2-trifluoroacetamide THF (15 mL) was added to crude N-(6-bromo-2-pyridinyl)-2,2,2-trifluoroacetamide (˜2.9 mmol). Pd(PPh₃)₂Cl₂ (88 mg, 0.125 mmol), CuI (47 mg, 0.25 mmol), TMS-acetylene (0.7 mL, 5.0 mmol), and TEA (1.05 mL, 7.5 mmol) were added and the mixture was heated at 40° C. for 30 min. Water (50 mL) was added and the mixture extracted with ethyl acetate (3×50 mL) followed by neutralizing with HCl (10 ml, 1.0M). The organic layer was washed with brine, dried with MgSO₄, filtered, and evacuated in vacuo. The crude material was dissolved in THF (10 mL) followed by addition of TBAF (3 mL, 1.0M). After 5 min., the solvent is removed in vacuo and the oil passed through a pad of silica gel with ethyl acetate as the eluent. This material was dried on a vacuum pump (RT, 1 torr) overnight.

c) N-(6-Bromo-2-pyridinyl)-2,2,2-trifluoroacetamide. 2-Bromo,6-aminopyridine (500 mg, 2.91 mmol) and DMAP (17 mg) were added to CH₂Cl₂ (12 mL) at RT. TFM (1.03 mL, 7.3 mmol) was added and the reaction mixture was allowed to stir overnight. The crude material was used in subsequent steps after removing the solvent in vacuo.

### Biological Data

Compounds of the present invention were tested for ErbB family protein tyrosine kinase inhibitory activity in substrate phosphorylation assays.

Enzyme Assays:

Compounds of the present invention were tested for EGFR, ErbB-2, and ErbB4 protein tyrosine kinase Inhibitory activity in substrate phosphorylation assays using enzymes purified from a baculovirus expression system. Reagent production was conducted essentially as described (Brignola, P. S., et al, (2002) J. Biol. Chem. v. 277 2, 1576-1585).

The method measures the ability of the isolated enzyme to catalyse the transfer of the γ-phosphate from ATP onto the tyrosine residue of a biotinylated synthetic peptide (biotin-Ahx-RAHEEIYHFFFAKKK-amide). The extent of tyrosine phosphorylation was measured using an anti-phosphotyrosine antibody, and quantified by homogenous time-resolved fluorescence (HTRF).

The enzymes were first diluted from their concentrated stock solutions into buffer containing 100 mM MOPS (pH7.5); 0.01% Tween-20; 0.1 mg/mL bovine serum albumin (BSA); and 80 nM EGFR, 100 nM ErbB2, or 100 nM ErbB4. The enzymes were incubated in this buffer for 30 minutes at room temperature before addition to the assay plates. Reactions were performed in black 384-well polystyrene flat-bottom plates in a final volume of 20 μL. Reaction mixtures contained 100 mM MOPS (pH 7.5), 2 mM MnCl₂, 20 μM ATP, 0.01% Tween-20, 0.1 mg/mL (BSA), 0.8 μM peptide substrate, and 1 mM dithiothreitol. Reactions were initiated by adding enzyme. 0.4 nM EGRF, 5 nM ErbB2, and 0.5 nM ErbB4 were the final enzyme concentrations.

Reactions were allowed to proceed for 90 minutes and were then terminated by the addition of 20 μL 100 mM EDTA to each well. 40 μL/well of HTRF mix were then added to the assay plates for the detection of phosphorylated substrate. Final assay concentrations were: 100 mM HEPES (pH7.5), 0.1 mg/mL BSA, 15 nM streptavidin-labeled allophycocyanin (PerkinElmer), and 1 nM europium-labeled anti-phosphotyrosine antibody (PerkinElmer). Assay plates were left unsealed and were counted in a Wallac Multilabel Counter 1420 (PerkinElmer).

Compounds under analysis were dissolved in Me₂SO to 1.0 mM and serially diluted 1 to 3 with Me₂SO through twelve dilutions. 1 μL of each concentration was transferred to the corresponding well of an assay plate. This creates a final compound concentration range from 0.00027 to 47.6 μM.

The data for dose responses were plotted as % Inhibition calculated with the data reduction formula 100*(1−(U1−C2)/(C1−C2)) versus concentration of compound where U is the unknown value, C1 is the average control value obtained for 4.76% DMSO, and C2 is the average control value obtained for 0.035 M EDTA. Data were fitted with a curve described by:
y=((Vmax*x)/(K+x))+Y2
where Vmax is the upper asymptote, Y2 is the Y intercept, and K is the IC50. The results for reach compound were recorded as pIC50s, calculated as follows:
pIC50=−Log10(K)

All exemplified Examples 1-100 were run with the recited assay and showed inhibitory activity with a pIC₅₀ of 5.0 or greater.

Results for specific Examples are depicted in Table I below.

TABLE I

| | EGFR Enzyme | ErbB2 Enzyme |
|---|---|---|
| Example 5 | + | + |
| Example 9 | ++ | ++ |
| Example 14 | +++ | +++ |
| Example 17 | +++ | +++ |
| Example 20 | +++ | +++ |
| Example 21 | +++ | +++ |
| Example 22 | +++ | +++ |
| Example 49 | ++ | +++ |
| Example 67 | +++ | +++ |
| Example 70 | +++ | +++ |
| Example 71 | ++ | ++ |
| Example 72 | + | + |
| Example 76 | ++ | ++ |
| Example 78 | +++ | +++ |
| Example 80 | + | + |
| Example 88 | +++ | +++ |
| Example 89 | +++ | +++ |
| Example 92 | +++ | +++ |
| Example 99 | +++ | +++ |
| Example 100 | +++ | +++ |

+ = pIC₅₀ < 6.0
++ = pIC₅₀ > 6.0 and < 7.0
+++ = pIC₅₀ > 7.0

What is claimed is:

1. A compound of Formula (I): 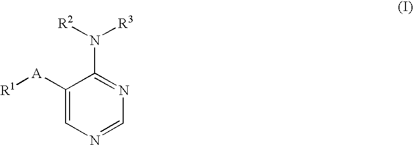
or a salt, solvate, or physiologically functional derivative thereof:
wherein:
A is C₁-C₄ alkenylene or C₁-C₄ alkynylene;
R is C₁-C₄ alkylene;
R¹ is the group defined by -(Z)-(Z¹)ₘ-(Z²)ₙ, wherein
Z is aryl, heteroaryl, heteroarylene, or arylene,
Z¹ is C(H)₂, where m is 0 or 1,
Z² is OR′, —SR′, —N(R′)R″, halo, C₁-C₃ alkyl, —CN, —C(O)R′, —C(O)N(R′)R″, or heterocyclyl, where n is 0 or 1;
R′ is —H or C₁-C₃ alkyl;
R″ is —H, —C(O)R′″, —C(S)R′″, —ROR′″, —C(═NH)N(R′)R″, C₁-C₃ alkyl, C₁-C₃ hydroxyalkyl, cyanoalkyl, —S(O)₂R′″, —RS(O)₂R′″, —C(O)N(R′)R′″, —C(O)N(R′)R^{a}, —C(O)RS(O)₂R′″, —C(O)ROROR′″, —C(O)RSR′″, —C(O)RNR′R″, —C(O)RC(O)OR′″, —RN(R′)RN(R′)C(O)R′″, heterocyclyl, or aralkyl;
R′″ is C₁-C₃ alkyl, C₁-C₃ haloalkyl, C₁-C₃ hydroxyalkyl, cyanoalkyl, or aryl;
R^{a} is —RS(O)₂R′, aralkyl, or —ROR′″;
R² is —H or C₁-C₃ alkyl;
R³ is the group defined by -(Q)-(Q¹)ᵣ-(Q²), wherein
Q is arylene or heteroarylene
Q¹ is O, where r is 0 or 1, and
Q² is aralkyl, heteroaryl, or aryl.

2. A compound of Formula (I): 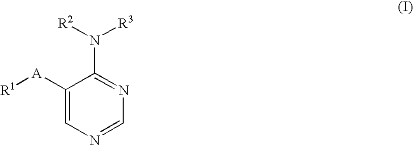
or a salt, solvate, or physiologically functional derivative thereof:
wherein:
A is C≡C;
R is C₁-C₄ alkylene;
R¹ is the group defined by -(Z)-(Z¹)ₘ-(Z²)ₙ, wherein
Z is aryl, heteroaryl, heteroarylene, or arylene,
Z¹ is C(H)₂, where m is 0 or 1,
Z² is OR′, —SR′, —N(R′)R″, halo, C₁-C₃ alkyl, —CN, —C(O)R′, —C(O)N(R′)R″, or heterocyclyl, where n is 0 or 1;
R′ is —H or C₁-C₃ alkyl;
R″ is —H, —C(O)R′″, —C(S)R′″, —ROR′″, —C(═NH)N(R′)R″, C₁-C₃ alkyl, C₁-C₃ hydroxyalkyl, cyanoalkyl, —S(O)₂R′″, —RS(O)₂R′″, —C(O)N(R′)R′″, —C(O)N(R′)R^{a}, —C(O)RS(O)₂R′″, —C(O)ROROR′″, —C(O)RSR′″, —C(O)RNR′R″, —C(O)RC(O)OR′″, —RN(R′)RN(R′)C(O)R′″, heterocyclyl, or aralkyl;
R′″ is C₁-C₃ alkyl, C₁-C₃ haloalkyl, C₁-C₃ hydroxyalkyl, cyanoalkyl, or aryl;
R^{a} is —RS(O)₂R′, aralkyl, or —ROR′″;
R² is —H or C₁-C₃ alkyl;
R³ is the group defined by -(Q)-(Q¹)ᵣ-(Q²), wherein
Q is arylene or heteroarylene
Q¹ is O, where r is 0 or 1, and
Q² is aralkyl, heteroaryl, or aryl.

3. A compound as claimed in claim 1, wherein A is C₁-C₄ alkynylene.

4. A compound as claimed in claim 1, wherein A is C≡C.

5. A compound as claimed in claim 1, wherein A is C₁-C₄ alkenylene.

6. A compound as claimed in claim 1, wherein A is C═C.

7. A compound as claimed in claim 1, wherein Z is heteroaryl and m and n are each 0.

8. A compound as claimed in claim 1, wherein Z is is selected from

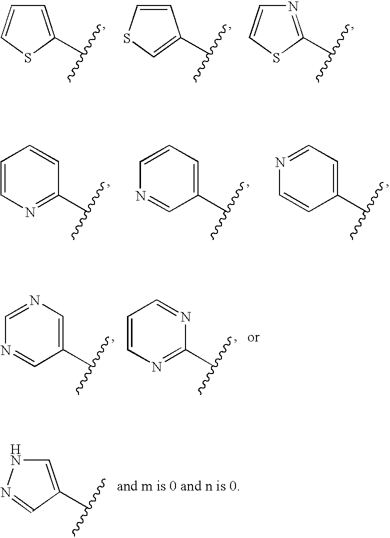

9. A compound as claimed in claim 1, wherein Z is heteroarylene, m is 0 or 1 and n is 1.

10. A compound as claimed in claim 1, wherein Z is selected from

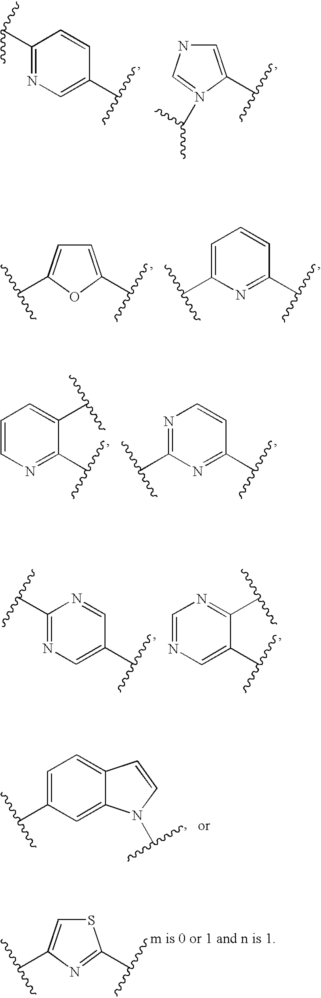

11. A compound as claimed in claim 1, wherein Z is aryl, and m and n are each 0.

12. A compound as claimed in claim 1, wherein Z is

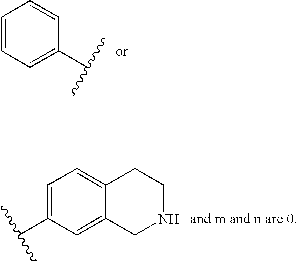

13. A compound as claimed in claim 1, wherein Z is arylene, m is 0 or 1 and n is 1.

14. A compound as claimed in claim 1, wherein Z is

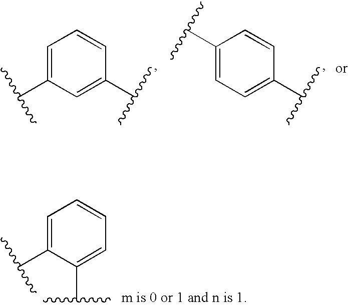

15. A compound as claimed in claim 1, wherein R² is —H.

16. A compound as claimed in claim 1, wherein R² is C₁-C₃ alkyl.

17-20. (canceled)

21. A compound as claimed in claim 1 selected from the group:
2-benzyl-N-{5-[(E)-2-phenylethenyl]pyrimidin-4-yl}-1H-benzimidazol-5-amine;
2-benzyl-N-{5-[(E)-2-thien-3-ylethenyl]pyrimidin-4-yl}-1H-benzimidazol-5-amine;
2-benzyl-N-{5-[(E)-2-(1H-pyrazol-4-yl)ethenyl]pyrimidin-4-yl}-1H-benzimidazol-5-amine;
3-((E)-2-{4-[(2-benzyl-1H-benzimidazol-5-yl)amino]pyrimidin-5-yl}ethenyl)-N-methylbenzamide;
2-benzyl-N-{5-[(E)-2-thien-3-ylethenyl]pyrimidin-4-yl}-1,3-benzothiazol-5-amine;
1-benzyl-N-{5-[(E)-2-pyridin-3-ylethenyl]pyrimidin-4-yl}-1H-indazol-5-amine;
1-benzyl-N-{5-[(E)-2-pyridin-4-ylethenyl]pyrimidin-4-yl}-1H-indazol-5-amine;
2-((E)-2-{4-[(1-benzyl-1H-indazol-5-yl)amino]pyrimidin-5-yl}ethenyl)pyridin-3-ol;
1-benzyl-N-{5-[(E)-2-(1H-pyrazol-4-yl)ethenyl]pyrimidin-4-yl}-1H-indazol-5-amine;
N-{5-[(E)-2-(2-aminopyrimidin-5-yl)ethenyl]pyrimidin-4-yl}-1-benzyl-1H-indazol-5-amine;
N-[3-((E)-2-{4-[(1-benzyl-1H-indazol-5-yl)amino]pyrimidin-5-yl}ethenyl)phenyl]acetamide;
N-(4-phenoxyphenyl)-5-[(E)-2-phenylethenyl]pyrimidin-4-amine;
N-{3-chloro-4-[(3-fluorobenzyl)oxy]phenyl}-5-[(E)-2-pyridin-3-ylethenyl]pyrimidin-4-amine;
N-{3-chloro-4-[(3-fluorobenzyl)oxy]phenyl}-5-[(E)-2-pyridin-4-ylethenyl]pyrimidin-4-amine;
2-{(E)-2-[4-({3-chloro-4-[(3-fluorobenzyl)oxy]phenyl}amino)pyrimidin-5-yl]ethenyl}pyridin-3-ol;
N-{3-chloro-4-[(3-fluorobenzyl)oxy]phenyl}-5-[(E)-2-thien-2-ylethenyl]pyrimidin-4-amine;
N-{3-chloro-4-[(3-fluorobenzyl)oxy]phenyl}-5-[(E)-2-thien-3-yletheny]pyrimidin-4-amine;
5-{(E)-2-[4-({3-chloro-4-[(3-fluorobenzyl)oxy]phenyl}amino)pyrimidin-5-yl]ethenyl}pyrimidin-2-amine;
N-{3-chloro-4-[(3-fluorobenzyl)oxy]phenyl}-5-[(E)-2-(1H-pyrazol-4-yl)ethenyl]pyrimidin-4-amine;
N-(3-{(E)-2-[4-({3-chloro-4-[(3-fluorobenzyl)oxy]phenyl}amino)pyrimidin-5-yl]ethenyl}phenyl)acetamide;
N-{3-chloro-4-[(3-fluorobenzyl)oxy]phenyl}-5-[(E)-2-(3,4-dimethoxyphenyl) ethenyl]pyrimidin-4-amine;
N-{3-chloro-4-[(3-fluorobenzyl)oxy]phenyl}-5-[(E)-2-phenylethenyl]pyrimidin-4-amine;
N-(5-{(E)-2-[4-({3-chloro-4-[(3-fluorobenzyl)oxy]phenyl}amino)pyrimidin-5-yl]ethenyl}pyridin-2-yl)acetamide;
N-{3-chloro-4-[(3-fluorobenzyl)oxy]phenyl}-5-(thien-2-ylethynyl)pyrimidin-4-amine;
N-{3-chloro-4-[(3-fluorobenzyl)oxy]phenyl}-5-(pyridin-3-ylethynyl)pyrimidin-4-amine;
N-{3-chloro-4-[(3-fluorobenzyl)oxy]phenyl}-5-[(1-methyl-1H-imidazol-5-yl)ethynyl]pyrimidin-4-amine;
N-{3-chloro-4-[(3-fluorobenzyl)oxy]phenyl}-5-(1H-pyrazol-4-ylethynyl)pyrimidin-4-amine;
N-{3-chloro-4-[(3-fluorobenzyl)oxy]phenyl}-5-(pyrimidin-5-ylethynyl)pyrimidin-4-amine;
N-{3-chloro-4-[(3-fluorobenzyl)oxy]phenyl}-5-(1,3-thiazol-2-ylethynyl)pyrimidin-4-amine;
N-{3-chloro-4-[(3-fluorobenzyl)oxy]phenyl}-5-(thien-3-ylethynyl)pyrimidin-4-amine;
N-{3-chloro-4-[(3-fluorobenzyl)oxy]phenyl}-5-[(2-morpholin-4-ylpyrimidin-4-yl)ethynyl]pyrimidin-4-amine;
N-(3-chloro-4-{[(3-fluorophenyl)methyl]oxy}phenyl)-5-(2-pyrimidinylethynyl)-4-pyrimidinamine;
5-[(6-amino-3-pyridinyl)ethynyl]-N-(3-chloro-4-{[(3-fluorophenyl)methyl]oxy}phenyl)-4-pyrimidinamine;
N-{3-chloro-4-[(3-fluorobenzyl)oxy]phenyl}-5-[(3-fluorophenyl)ethynyl]pyrimidin-4-amine;
4-{[4-({3-chloro-4-[(3-fluorobenzyl)oxy]phenyl}amino)pyrimidin-5-yl]ethynyl}phenol;
N-{3-chloro-4-[(3-fluorobenzyl)oxy]phenyl}-5-[(6-methoxypyridin-2-yl)ethynyl]pyrimidin-4-amine;
5-[(3-aminophenyl)ethynyl]-N-{3-chloro-4-[(3-fluorobenzyl)oxy]phenyl}pyrimidin-4-amine;
N-(3-{[4-({3-chloro-4-[(3-fluorobenzyl)oxy]phenyl}amino)pyrimidin-5-yl]ethynyl}phenyl)acetamide;
N-(3-{[4-({3-chloro-4-[(3-fluorobenzyl)oxy]phenyl}amino)pyrimidin-5-yl]ethynyl}phenyl)ethanethioamide;
2-{[4-({3-chloro-4-[(3-fluorobenzyl)oxy]phenyl}amino)pyrimidin-5-yl]ethynyl}benzonitrile;
3-{[4-({3-chloro-4-[(3-fluorobenzyl)oxy]phenyl}amino)pyrimidin-5-yl]ethynyl}benzonitrile;
3-{[4-({3-chloro-4-[(3-fluorobenzyl)oxy]phenyl}amino)pyrimidin-5-yl]ethynyl}benzaldehyde;
N-{3-chloro-4-[(3-fluorobenzyl)oxy]phenyl}-5-(phenylethynyl)pyrimidin-4-amine;
N-{3-chloro-4-[(3-fluorobenzyl)oxy]phenyl}-5-(pyridin-2-ylethynyl)pyrimidin-4-amine;
5-[(4-aminophenyl)ethynyl]-N-{3-chloro-4-[(3-fluorobenzyl)oxy]phenyl}pyrimidin-4-amine;
N-(3-{[4-({3-chloro-4-[(3-fluorobenzyl)oxy]phenyl}amino)pyrimidin-5-yl]ethynyl}phenyl)-3-(methylthio)propanamide;
N-{3-chloro-4-[(3-fluorobenzyl)oxy]phenyl}-5-({1-[(4-methylphenyl)sulfonyl]-1H-indol-6-yl}ethynyl)pyrimidin-4-amine;
tert-butyl-3-{[4-({3-chloro-4-[(3-fluorobenzyl)oxy]phenyl}amino)pyrimidin-5-yl]ethynyl}benzylcarbamate;
N-(3-{[4-({3-chloro-4-[(3-fluorobenzyl)oxy]phenyl}amino)pyrimidin-5-yl]ethynyl}phenyl)guanidine;
N-(3-{[4-({3-chloro-4-[(3-fluorobenzyl)oxy]phenyl}amino)pyrimidin-5-yl]ethynyl}benzyl)acetamide;
N-{3-chloro-4-[(3-fluorobenzyl)oxy]phenyl}-5-{[3-({[2-(methylsulfonyl) ethyl]amino}methyl)phenyl]ethynyl}pyrimidin-4-amine;
5-{[4-({3-chloro-4-[(3-fluorobenzyl)oxy]phenyl}amino)pyrimidin-5-yl]ethynyl}-2-furaldehyde;
3-{[(5-{[4-({3-chloro-4-[(3-fluorobenzyl)oxy]phenyl}amino)pyrimidin-5-yl]ethynyl}-2-furyl)methyl]amino}propanenitrile;
(5-{[4-({3-chloro-4-[(3-fluorobenzyl)oxy]phenyl}amino)pyrimidin-5-yl]ethynyl}-2-furyl)methanol;
(4-{[4-({3-chloro-4-[(3-fluorobenzyl)oxy]phenyl}amino)pyrimidin-5-yl]ethynyl}-1,3-thiazol-2-yl)methanol;
N-{3-chloro-4-[(3-fluorobenzyl)oxy]phenyl}-5-(1,2,3,4-tetrahydro-isoquinolin-7-ylethynyl)pyrimidin-4-amine;
2-{[4-({3-chloro-4-[(3-fluorobenzyl)oxy]phenyl}amino)pyrimidin-5-yl]ethynyl}benzaldehyde;
N-{3-chloro-4-[(3-fluorobenzyl)oxy]phenyl}-5-{[5-({[2-(methylsulfonyl)ethyl]amino}methyl)-2-furyl]ethynyl}pyrimidin-4-amine;
N-(3-{[4-({3-chloro-4-[(3-fluorobenzyl)oxy]phenyl}amino)pyrimidin-5-yl]ethynyl}phenyl)-2-(2-methoxyethoxy)acetamide;
N-[3-({4-[(2-benzyl-1H-benzimidazol-5-yl)amino]pyrimidin-5-yl}ethynyl)phenyl]acetamide;
N¹-(3-{[4-({3-chloro-4-[(3-fluorobenzyl)oxy]phenyl}amino)pyrimidin-5-yl]ethynyl}phenyl)-α-alaninamide;
N-(3-{[4-({3-chloro-4-[(3-fluorobenzyl)oxy]phenyl}amino)pyrimidin-5-yl]ethynyl}phenyl)-2-(methylsulfonyl)acetamide;
N-[3-({4-[(4-benzylphenyl)amino]pyrimidin-5-yl}ethynyl)phenyl]-acetamide;
N-[3-({4-[(4-phenoxyphenyl)amino]pyrimidin-5-yl}ethynyl)phenyl]acetamide;
N-[3-({4-[(1-benzyl-1H-indazol-5-yl)amino]pyrimidin-5-yl}ethynyl)phenyl]acetamide;
1-benzyl-N-[5-(phenylethynyl)pyrimidin-4-yl]-1H-indol-5-amine;
5-[(6-amino-2-pyridinyl)ethynyl]-N-(3-chloro-4-{[(3-fluorophenyl) methyl]oxy}phenyl)-4-pyrimidinamine;
N-{6-[2-(4-{3-chloro-4-[(3-fluorobenzyl)oxy]anilino}-5-pyrimidinyl)ethynyl]-2-pyridinyl}acetamide;
2-chloro-N-{6-[(4-{3-chloro-4-[(3-fluorobenzyl)oxy]anilino}-5-pyrimidinyl)ethynyl]-2-pyridinyl}-2,2-difluoroacetamide;
N-{6-[(4-{3-chloro-4-[(3-fluorobenzyl)oxy]anilino}-5-pyrimidinyl)ethynyl]-2-pyridinyl}-4-(dimethylamino)butanamide;
methyl 4-({6-[(4-{3-chloro-4-[(3-fluorobenzyl)oxy]anilino}-5-pyrimidinyl)ethynyl]-2-pyridinyl}amino)-4-oxobutanoate;
N-{3-chloro-4-[(3-fluorobenzyl)oxy]phenyl}-5-[(6-{[2-(methylsulfonyl)ethyl]amino}-2-pyridinyl)ethynyl]-4-pyrimidinamine;
{6-[(4-{3-chloro-4-[(3-fluorobenzyl)oxy]anilino}-5-pyrimidinyl)ethynyl]-2-pyridinyl}methanol;
2-[({6-[(4-{3-chloro-4-[(3-fluorobenzyl)oxy]anilino}-5-pyrimidinyl)ethynyl]-2-pyridinyl}methyl)(methyl)amino]ethanol;
3-[({6-[(4-{3-chloro-4-[(3-fluorobenzyl)oxy]anilino}-5-pyrimidinyl)ethynyl]-2-pyridinyl}methyl)amino]propanenitrile;
N-{3-chloro-4-[(3-fluorobenzyl)oxy]phenyl}-5-{[6-({[2-(4-morpholinyl)ethyl]amino}methyl)-2-pyridinyl]ethynyl}-4-pyrimidinamine;
N-{2-[({6-[2-(4-{3-chloro-4-[(3-fluorobenzyl)oxy]anilino}-5-pyrimidinyl)ethynyl]-2-pyridinyl}methyl)amino]ethyl}acetamide;
N-{3-chloro-4-[(3-fluorobenzyl)oxy]phenyl}-5-{[6-({[3-(1H-imidazol-1-yl)propyl]amino}methyl)-2-pyridinyl]ethynyl}-4-pyrimidinamine;
N-{3-chloro-4-[(3-fluorobenzyl)oxy]phenyl}-5-({6-[(methylamino)methyl]-2-pyridinyl}ethynyl)-4-pyrimidinamine;
N-{3-chloro-4-[(3-fluorobenzyl)oxy]phenyl}-5-{[6-(methoxymethyl)-2-pyridinyl]ethynyl}-4-pyrimidinamine;
N-{3-chloro-4-[(3-fluorobenzyl)oxy]phenyl}-5-{[2-(methylsulfanyl)-4-pyrimidinyl]ethynyl}-4-pyrimidinamine;
N-{3-chloro-4-[(3-fluorobenzyl)oxy]phenyl}-5-({6-[(dimethylamino)methyl]-2-pyridinyl}ethynyl)-4-pyrimidinamine;
N-benzyl-N-({6-[(4-{3-chloro-4-[(3-fluorobenzyl)oxy]anilino}-5-pyrimidinyl)ethynyl]-2-pyridinyl}methyl)amine;
N-{3-chloro-4-[(3-fluorobenzyl)oxy]phenyl}-5-[(6-{[(2-methoxyethyl)amino]methyl}-2-pyridinyl)ethynyl]-4-pyrimidinamine;
5-{[6-(aminomethyl)-2-pyridinyl]ethynyl}-N-{3-chloro-4-[(3-fluorobenzyl) oxy]phenyl}-4-pyrimidinamine;
N-({6-[(4-{3-chloro-4-[(3-fluorobenzyl)oxy]anilino}-5-pyrimidinyl)ethynyl]-2-pyridinyl}methyl)-N′-(2-cyanoethyl)urea;
N′({6-[(4-{3-chloro-4-[(3-fluorobenzyl)oxy]anilino}-5-pyrimidinyl)ethynyl]-2-pyridinyl}methyl)-N-(2-hydroxyethyl)-N-methylurea;
N-({6-[(4-{3-chloro-4-[(3-fluorobenzyl)oxy]anilino}-5-pyrimidinyl)ethynyl]-2-pyridinyl}methyl)-N′-[2-(methylsulfonyl)ethyl]urea;
N-({6-[(4-{3-chloro-4-[(3-fluorobenzyl)oxy]anilino}-5-pyrimidinyl)ethynyl]-2-pyridinyl}methyl)-N′-[2-(4-morpholinyl)ethyl]urea;
N-({6-[(4-{3-chloro-4-[(3-fluorobenzyl)oxy]anilino}-5-pyrimidinyl)ethynyl]-2-pyridinyl}methyl)-N′-methylurea;
N-({6-[(4-{3-chloro-4-[(3-fluorobenzyl)oxy]anilino}-5-pyrimidinyl)ethynyl]-2-pyridinyl}methyl)-N′-(2-methoxyethyl)urea;
N-{3-chloro-4-[(3-fluorobenzyl)oxy]phenyl}-5-{[6-(1-piperidinylmethyl)-2-pyridinyl]ethynyl}-4-pyrimidinamine;
N-{3-chloro-4-[(3-fluorobenzyl)oxy]phenyl}-5-({6-[(4-methyl-1-piperazinyl)methyl]-2-pyridinyl}ethynyl)-4-pyrimidinamine;
N-{3-chloro-4-[(3-fluorobenzyl)oxy]phenyl}-5-{[6-(4-morpholinyl-methyl)-2-pyridinyl]ethynyl}-4-pyrimidinamine;
N-{3-chloro-4-[(3-fluorobenzyl)oxy]phenyl}-5-{[6-(1-pyrrolidinyl-methyl)-2-pyridinyl]ethynyl}-4-pyrimidinamine;
N-{3-chloro-4-[(3-fluorobenzyl)oxy]phenyl}-5-{[6-(1-piperazinylmethyl)-2-pyridinyl]ethynyl}-4-pyrimidinamine;
4-amino-2-{[4-({3-chloro-4-[(4-fluorobenzyl)oxy]phenyl}amino)pyrimidin-5-yl]ethynyl}pyrimidine-5-carbonitrile;
2-{[4-({3-chloro-4-[(3-fluorobenzyl)oxy]phenyl}amino)pyrimidin-5-yl]ethynyl}-4-{[2-(methylsulfonyl)ethyl]amino}pyrimidine-5-carbonitrile;
4-{[4-({3-chloro-4-[(3-fluorobenzyl)oxy]phenyl}amino)pyrimidin-5-yl]ethynyl}pyrimidin-2-amine; and
N-(6-{[4-({3-chloro-4-[(3-fluorobenzyl)oxy]phenyl}amino)pyrimidin-5-yl]ethynyl}pyridin-2-yl)-2,2,2-trifluoroacetamide;
or a salt, solvate, or physiologically functional derivative thereof.

22. A pharmaceutical composition, comprising: a therapeutically effective amount of a compound as claimed in any one of claim 1, or a salt, solvate, or a physiologically functional derivative thereof and one or more of pharmaceutically acceptable carriers, diluents and excipients.

23. A method of treating a disorder in a mammal, said disorder being mediated by inappropriate activity of at least one erbB family kinase, comprising: administering to said mammal a therapeutically effective amount of a compound as claimed in claim 1, or a salt, solvate or a physiologically functional derivative thereof.

24. A method of treating a disorder in a mammal, said disorder being mediated by inappropriate activity of at least two erbB family kinases, comprising: administering to said mammal a therapeutically effective amount of a compound as claimed in any one of claim 1, or a salt, solvate or a physiologically functional derivative thereof.

25-27. (canceled)
